(19) United States
(12) Patent Application Publication
(10) Pub. No.: US 2008/0306076 A1
(43) Pub. Date: Dec. 11, 2008

(54) MODULATION OF CHEMOSENSORY RECEPTORS AND LIGANDS ASSOCIATED THEREWITH

(75) Inventors: Xiaodong Li, San Diego, CA (US); Feng Zhang, San Diego, CA (US); Hong Xu, San Diego, CA (US); Catherine Tachdjian, San Diego, CA (US); Sara Werner, San Diego, CA (US); Andrew Patron, San Marcos, CA (US); Albert Zlotnik, San Diego, CA (US); Mark Zoller, La Jolla, CA (US); Boris Klebansky, Demarest, NJ (US); Richard Fine, Ridgewood, NJ (US); Xinshan Kang, Pine Brook, NJ (US)

Correspondence Address: COOLEY GODWARD KRONISH LLP;ATTN: Patent Group, Suite 1100, 777 - 6th Street, NW, WASHINGTON, DC, 20001, US

(73) Assignee: SENOMYX, INC., San Diego, CA (US)

(21) Appl. No.: 11/760,666

(22) Filed: Jun. 8, 2007

(51) Int. Cl. A61K 31/495; A61P 27/00; C07D 487/00; C12N 5/06; G01N 33/53; G06G 7/48

(52) U.S. Cl. 514/249; 435/375; 435/7.1; 544/236; 703/11

(57) ABSTRACT

The present invention provides screening methods for identifying modifiers of chemosensory receptors and their ligands, e.g., by determining whether a test entity is suitable to interact with one or more interacting sites within the Venus flytrap domains of the chemosensory receptors as well as modifiers capable of modulating chemosensory receptors and their ligands.

### BACKGROUND OF THE INVENTION

The taste system provides sensory information about the chemical composition of the external world. Taste transduction is one of the most sophisticated forms of chemical-triggered sensation in animals. Signaling of taste is found throughout the animal kingdom, from simple metazoans to the most complex of vertebrates. Sensations associated with taste are thought to involve distinct signaling pathways mediated by receptors, i.e., metabotropic or ionotropic receptors. Cells which express taste receptors, when exposed to certain chemical stimuli, elicit taste sensation by depolarizing to generate an action potential, which is believed to trigger the sensation. This event is believed to trigger the release of neurotransmitters at gustatory afferent neuron synapses, thereby initiating signaling along neuronal pathways that mediate taste perception.

As such, taste receptors specifically recognize molecules that elicit specific taste sensation. These molecules are also referred to herein as “tastants.” Many taste receptors belong to the 7-transmembrane receptor superfamily, which are also known as G protein-coupled receptors (GPCRs). Other tastes are believed to be mediated by channel proteins. G protein-coupled receptors control many physiological functions, such as endocrine function, exocrine function, heart rate, lipolysis, carbohydrate metabolism, and transmembrane signaling.

For example, family C of G-protein coupled receptors (GPCRs) from humans comprise eight metabotropic glutamate (mGlu(1-8)) receptors, two heterodimeric gamma-aminobutyric acid(B) (GABA(B)) receptors, a calcium-sensing receptor (CaR), three taste (T1R) receptors, a promiscuous L-alpha-amino acid receptor (GPRC6A), and five orphan receptors. The family C GPCRs are characterized by a large amino-terminal domain, which bind the endogenous orthosteric agonists. Additionally, allosteric modulators which bind to the seven transmembrane domains of the receptors have also been reported.

In general, upon ligand binding to a GPCR, the receptor presumably undergoes a conformational change leading to activation of a G protein. G proteins are comprised of three subunits: a guanyl nucleotide binding α-subunit, a β-subunit, and a γ-subunit. G proteins cycle between two forms, depending on whether GDP or GTP is bound to the α-subunit. When GDP is bound, the G protein exists as a heterotrimer: the G_{α-β-γ} complex. When GTP is bound, the α-subunit dissociates from the heterotrimer, leaving a G_{β-γ} complex. When a G_{α-β-γ} complex operatively associates with an activated G protein-coupled receptor in a cell membrane, the rate of exchange of GTP for bound GDP is increased and the rate of dissociation of the bound Gα subunit from the G_{α-β-γ} complex increases. The free G_{α} subunit and G_{β-γ} complex are thus capable of transmitting a signal to downstream elements of a variety of signal transduction pathways. These events form the basis for a multiplicity of different cell signaling phenomena, including for example the signaling phenomena that are identified as neurological sensory perceptions such as taste and/or smell.

Mammals are believed to have five basic taste modalities: sweet, bitter, sour, salty, and umami (the taste of monosodium glutamate). Numerous physiological studies in animals have shown that taste receptor cells may selectively respond to different chemical stimuli. In mammals, taste receptor cells are assembled into taste buds that are distributed into different papillae in the tongue epithelium. Circumvallate papillae, found at the very back of the tongue, contain hundreds to thousands of taste buds. By contrast, foliate papillae, localized to the posterior lateral edge of the tongue, contain dozens to hundreds of taste buds. Further, fungiform papillae, located at the front of the tongue, contain only a single or a few taste buds.

Each taste bud, depending on the species, contains 50-150 cells, including precursor cells, support cells, and taste receptor cells. Receptor cells are innervated at their base by afferent nerve endings that transmit information to the taste centers of the cortex through synapses in the brain stem and thalamus. Elucidating the mechanisms of taste cell signaling and information processing is important to understanding the function, regulation, and perception of the sense of taste.

The gustatory system has been selected during evolution to detect nutritive and beneficial compounds as well as harmful or toxic substances. Outside the tongue, expression of Gα_{gust} has also been localized to gastric and pancreatic cells, suggesting that a taste-sensing mechanism may also exist in the gastrointestinal (GI) tract. Expression of taste receptors has also been found in the lining of stomach and intestine, suggesting that taste receptors may play a role in molecular sensing of therapeutic entities and toxins.

Complete or partial sequences of numerous human and other eukaryotic chemosensory receptors are currently known. Within the last several years, a number of groups including the present assignee Senomyx, Inc. have reported the identification and cloning of genes from two GPCR families that are involved in taste modulation and have obtained experimental results related to the understanding of taste biology. These results indicate that bitter, sweet and amino acid taste, also referred as umami taste, are triggered by activation of two types of specific receptors located at the surface of taste receptor cells (TRCs) on the tongue i.e., T2Rs and T1Rs. It is currently believed that at least 26 to 33 genes encode functional receptors (T2R5) for bitter tasting substances in human and rodent respectively.

By contrast there are only 3 T1Rs, T1R1, T1R2 and T1R3, which are involved in umami and sweet taste. Structurally, the T1R and T2R receptors possess the hallmark of G protein-coupled receptors (GPCRs), i.e., 7 transmembrane domains flanked by small extracellular and intracellular amino- and carboxyl-termini respectively.

T2Rs have been cloned from different mammals including rats, mice and humans. T2Rs comprise a novel family of human and rodent G protein-coupled receptors that are expressed in subsets of taste receptor cells of the tongue and palate epithelia. These taste receptors are organized in clusters in taste cells and are genetically linked to loci that influence bitter taste. The fact that T2Rs modulate bitter taste has been demonstrated in cell-based assays. For example, mT2R-5, hT2R-4 and mT2R-8 have been shown to be activated by bitter molecules in in vitro gustducin assays, providing experimental proof that T2Rs function as bitter taste receptors.

T1R family members in general include T1R1, T1R2, and T1R3, e.g., rT1R3, mT1R3, hT1R3, rT1R2, mT1R2, hT1R2, and rT1R1, mT1R1 and hT1R1. It is known that the three T1R gene members T1R1, T1R2 and T1R3 form functional heterodimers that specifically recognize sweeteners and amino acids. It is generally believed that T1R2/T1R3 combination recognizes natural and artificial sweeteners while the T1R1/T1R3 combination recognizes several L-amino acids and monosodium glutamate (MSG), respectively. For example, co-expression of T1R1 and T1R3 in recombinant host cells results in a hetero-oligomeric taste receptor that responds to umami taste stimuli. Umami taste stimuli include by way of example monosodium glutamate and other molecules that elicit a “savory” taste sensation. By contrast, co-expression of T1R2 and T1R3 in recombinant host cells results in a hetero-oligomeric sweet taste receptor that responds to both naturally occurring and artificial sweeteners.

There is a need in the art to develop various ways of identifying compounds or other entities suitable for modifying receptors and their ligands associated with chemosensory or chemosensory related sensation or reaction. In addition, there is a need in the art for compounds or other entities with such characteristics.

### BRIEF SUMMARY OF THE INVENTION

The present invention is based, at least in part, on the discovery that an extra-cellular domain, e.g., a Venus flytrap domain of a chemosensory receptor, especially one or more interacting sites within the Venus flytrap domain is a suitable target for compounds or other entities to modulate the chemosensory receptor and/or its ligands. Accordingly, the present invention provides screening methods for identifying modifiers of chemosensory receptors and their ligands as well as modifiers capable of modulating chemosensory receptors and their ligands.

In one embodiment, the present invention provides a method of screening for a candidate of a chemosensory receptor ligand modifier. The method comprises determining whether a test entity is suitable to interact with a chemosensory receptor via a first interacting site within the Venus flytrap domain of the chemosensory receptor.

In another embodiment, the present invention provides a method of screening for a candidate of a chemosensory receptor ligand modifier. The method comprises determining whether a test entity is suitable to interact with a chemosensory receptor via a first interacting site within the Venus flytrap domain of the chemosensory receptor, wherein the first interacting site is identified in light of a second interacting site identified based on the interaction between a chemosensory receptor ligand and the chemosensory receptor.

In yet another embodiment, the present invention provides a method of screening for a candidate of a chemosensory receptor modifier. The method comprises determining whether a test entity is suitable to interact with a chemosensory receptor via an interacting site within the Venus flytrap domain of the chemosensory receptor, wherein the interacting site includes an interacting residue selected from the group consisting of D147, S148, T149, N150, A170, A171, S172, S173, D192, N195, D218, Y220, S276, R277, E301, and A302 of a human T1R1 and a combination thereof, wherein a test entity suitable to interact with the interacting site of the chemosensory receptor is indicative of a candidate of a chemosensory receptor modifier.

In still another embodiment, the present invention provides a method of modulating the activity of a chemosensory receptor ligand. The method comprises contacting a chemosensory receptor ligand modifier with a cell containing T1R1Venus flytrap domain in the presence of a chemosensory receptor ligand, wherein the chemosensory receptor ligand modifier interacts with an interacting site of the chemosensory receptor.

In still yet another embodiment, the present invention provides a chemosensory receptor ligand modifier, wherein in the presence of a chemosensory receptor ligand it interacts with T1R1Venus flytrap domain via at least three interacting residues selected from the group consisting of L46, H47, S48, G49, C50, L51, S67, F68, N69, E70, H71, C106, S107, D108, D147, S148, T149, N150, R151, Y169, A170, A171, S172, S173, D192, N195, D218, Y220, S276, R277, E301, A302, F247, S248, S275, S276, R277, Q278, L279, A280, R281, V282, F283, F284, E285, E301, A302, W303, S306, R307, H308, I309, T310, G311, V312, P313, R317, K354, W357, K377, K379, M383, and S385 of a human T1R1.

### BRIEF DESCRIPTION OF THE FIGURES

FIG. 1 contains exemplary human T1R1 polymorphic variations.

FIG. 2 contains exemplary human T1R2 polymorphic variations.

FIG. 3 shows the dendograms for the sequence alignments of T1R1.

FIG. 4 shows the dendograms for the sequence alignments of T1R2.

FIG. 5 shows exemplary interacting spaces for monosodium glutamate and IMP.

FIG. 6 shows exemplary interacting spaces and residues for monosodium glutamate.

FIG. 7 shows exemplary interacting spaces and residues for IMP.

FIG. 8 shows exemplary interacting spaces and residues for monosodium glutamate and IMP (front in this view)

FIG. 9 shows exemplary interacting spaces and residues for monosodium glutamate and IMP (left in this view).

FIG. 10 shows exemplary interacting spaces and residues for monosodium glutamate and IMP (front in this view).

FIG. 11 shows activity against L-Glu for S172A, DI 192A, Y220A, and E301A mutants.

FIG. 12 shows results for exemplary mutagenesis studies.

FIG. 13 shows activity of IMP for wild type human umami receptor.

FIG. 14 shows activity against L-Glu for R277A, H308A, H71A, and S306A mutants.

FIG. 15 shows activity against L-Glu for H308E mutant.

### DETAILED DESCRIPTION OF THE INVENTION

Prior to specifically describing the invention, the following definitions are provided.

The term “T1R” family includes polymorphic variants, alleles, mutants, and homologs that: (1) have about 30-40% amino acid sequence identity, more specifically about 40, 50, 60, 70, 75, 80, 85, 90, 95, 96, 97, 98, or 99% amino acid sequence identity to the T1Rs known or disclosed, e.g., in patent application U.S. Ser. No. 10/179,373 filed on Jun. 26, 2002, U.S. Ser. No. 09/799,629 filed on Apr. 5, 2001 and U.S. Ser. No. 10/035,045 filed on Jan. 3, 2002, over a window of about 25 amino acids, optimally 50-100 amino acids; (2) specifically bind to antibodies raised against an immunogen comprising an amino acid sequence selected from the group consisting of the T1R sequences disclosed infra, and conservatively modified variants thereof, (3) specifically hybridize (with a size of at least about 100, optionally at least about 500-1000 nucleotides) under stringent hybridization conditions to a sequence selected from the group consisting of the T1R DNA sequences disclosed infra, and conservatively modified variants thereof, (4) comprise a sequence at least about 40% identical to an amino acid sequence selected from the group consisting of the T1R amino acid sequences disclosed infra or (5) are amplified by primers that specifically hybridize under stringent hybridization conditions to the described T1R sequences.

In particular, these “T1Rs” include taste receptor GPCRs referred to as hT1R1, hT1R2, hT1R3, rT1R1, rT1R2, rT1R3, mT1R1, mT1R2, and mT1R3 having the nucleic acid sequences and amino acid sequences known or disclosed, e.g., in U.S. Ser. No. 10/179,373 filed on Jun. 26, 2002, U.S. Ser. No. 09/799,629 filed on Apr. 5, 2001 and U.S. Ser. No. 10/035,045 filed on Jan. 3, 2002, and variants, alleles, mutants, orthologs and chimeras thereof which specifically bind and/or respond to sweet, umami, or any other chemosensory related ligands including activators, inhibitors and enhancers. Also T1Rs include taste receptor GPCRs expressed in humans or other mammals, e.g., cells associated with taste and/or part of gastrointestinal system including without any limitation, esophagus, stomach, intestine (small and large), colon, liver, biliary tract, pancreas, gallbladder, etc. Also, T1R polypeptides include chimeric sequences derived from portions of a particular T1R polypeptide such as T1R1, T1R2 or T1R3 of different species or by combining portions of different T1R5 wherein such chimeric T1R sequences are combined to produce a functional sweet or umami taste receptor. For example chimeric T1Rs may comprise the extracellular region of one T1R, i.e., T1R1 or T1R2 and the transmembrane region of another T1R, either T1R1 or T1R2.

Topologically, certain chemosensory GPCRs have an “N-terminal domain;” “extracellular domains,” a “transmembrane domain” comprising seven transmembrane regions, and corresponding cytoplasmic and extracellular loops, “cytoplasmic regions,” and a “C-terminal region” (see, e.g., Hoon et al., Cell, 96:541-51 (1999); Buck et al., Cell, 65:175-87 (1991)). These regions can be structurally identified using methods known to those of skill in the art, such as sequence analysis programs that identify hydrophobic and hydrophilic domains (see, e.g., Stryer, Biochemistry, (3rd ed. 1988); see also any of a number of Internet based sequence analysis programs, such as those found at dot.imgen.bcm.tmc.edu). These regions are useful for making chimeric proteins and for in vitro assays of the invention, e.g., ligand binding assays.

“Extracellular domains” therefore refers to the domains of chemosensory receptors, e.g., T1R polypeptides that protrude from the cellular membrane and are exposed to the extracellular face of the cell. Such regions would include the “N-terminal domain” that is exposed to the extracellular face of the cell, as well as the extracellular loops of the transmembrane domain that are exposed to the extracellular face of the cell, i.e., the extracellular loops between transmembrane regions 2 and 3, transmembrane regions 4 and 5, and transmembrane regions 6 and 7. The “N-terminal domain” starts at the N-terminus and extends to a region close to the start of the transmembrane region. These extracellular regions are useful for in vitro ligand binding assays, both soluble and solid phase. In addition, transmembrane regions, described below, can also be involved in ligand binding, either in combination with the extracellular region or alone, and are therefore also useful for in vitro ligand binding assays.

“Transmembrane domain,” which comprises the seven transmembrane “regions,” refers to the domains of chemosensory receptors, e.g., T1R polypeptides that lie within the plasma membrane, and may also include the corresponding cytoplasmic (intracellular) and extracellular loops, also referred to as transmembrane “regions.” The seven transmembrane regions and extracellular and cytoplasmic loops can be identified using standard methods, as described in Kyte et al., J. Mol. Biol. 157:105-32 (1982), or in Stryer, supra.

“Cytoplasmic domains” refers to the domains of chemosensory receptors, e.g., T1R proteins that face the inside of the cell, e.g., the “C-terminal domain” and the intracellular loops of the transmembrane domain, e.g., the intracellular loops between transmembrane regions 1 and 2, transmembrane regions 3 and 4, and transmembrane regions 5 and 6. “C-terminal domain” refers to the region that spans from the end of the last transmembrane region to the C-terminus of the protein, and which is normally located within the cytoplasm.

The term “7-transmembrane receptor” means a polypeptide belonging to a superfamily of transmembrane proteins that have seven regions that span the plasma membrane seven times (thus, the seven regions are called “transmembrane” or “TM” domains TM I to TM VII).

The phrase “functional effects” or “activity” in the context of the disclosed assays for testing compounds that modulate a chemosensory receptor, e.g., enhance T1R family member mediated signal transduction such as sweet or umami receptor functional effects or activity includes the determination of any parameter that is indirectly or directly under the influence of the particular chemosensory receptor, e.g., functional, physical and chemical effects. It includes, without any limitation, ligand binding, changes in ion flux, membrane potential, current flow, transcription, G protein binding, GPCR phosphorylation or dephosphorylation, signal transduction, receptor-ligand interactions, second messenger concentrations (e.g., cAMP, cGMP, IP3, or intracellular Ca²⁺), in vitro, in vivo, and ex vivo and also includes other physiologic effects such increases or decreases of neurotransmitter or hormone release.

The term “determining the functional effect” or receptor “activity” means assays for a compound that increases or decreases a parameter that is indirectly or directly under the influence of a chemosensory receptor, e.g., functional, physical and chemical effects. Such functional effects can be measured by any means known to those skilled in the art, e.g., changes in spectroscopic characteristics (e.g., fluorescence, absorbance, refractive index), hydrodynamic (e.g., shape), chromatographic, or solubility properties, patch clamping, voltage-sensitive dyes, whole cell currents, radioisotope efflux, inducible markers, oocyte chemosensory receptor, e.g., T1R gene expression; tissue culture cell chemosensory receptor, e.g., T1R expression; transcriptional activation of chemosensory receptor, e.g., T1R genes; ligand binding assays; voltage, membrane potential and conductance changes; ion flux assays; changes in intracellular second messengers such as cAMP, cGMP, and inositol triphosphate (IP3); changes in intracellular calcium levels; neurotransmitter release, and the like.

“Inhibitors,” “activators,” and “modifiers” of chemosensory receptor, e.g., T1R proteins are used interchangeably to refer to inhibitory, activating, or modulating molecules identified using in vitro and in vivo assays for chemosensory signal transduction, e.g., ligands, agonists, antagonists, and their homologs and mimetics. Inhibitors are compounds that, e.g., bind to, partially or totally block stimulation, decrease, prevent, delay activation, inactivate, desensitize, or down regulate taste transduction, e.g., antagonists. Activators are compounds that, e.g., bind to, stimulate, increase, open, activate, facilitate, enhance activation, sensitize, or up regulate chemosensory signal transduction, e.g., agonists. Modifiers include compounds that, e.g., alter the activity of a receptor or the interaction of a receptor with extracellular proteins, e.g., receptor ligands and optionally bind to or interact with activators or inhibitor; G Proteins; kinases (e.g., homologs of rhodopsin kinase and beta adrenergic receptor kinases that are involved in deactivation and desensitization of a receptor); and arrestins, which also deactivate and desensitize receptors. Modifiers include genetically modified versions of chemosensory receptors, e.g., T1R family members, e.g., with altered activity, as well as naturally occurring and synthetic ligands, antagonists, agonists, small chemical molecules and the like. In the present invention this includes, without any limitation, sweet ligands (agonists or antagonists), umami ligands (agonists and antagonists), sweet enhancers and umami enhancers and sweet taste or umami taste inhibitors.

“Enhancer” herein refers to a compound that modulates (increases) the activation of a particular receptor, preferably the chemosensory, e.g., T1R2/T1R3 receptor or T1R1/T1R3 receptor but which by itself does not result in substantial activation of the particular receptor. Herein such enhancers will enhance the activation of a chemosensory receptor by its ligand. Typically the “enhancer” will be specific to a particular ligand, i.e., it will not enhance the activation of a chemosensory receptor by chemosensory ligands other than the particular chemosensory ligand or ligands closely related thereto.

“Putative enhancer” herein refers to a compound identified, e.g., in silico or not, as a potential enhancer using assays which are described herein but which enhancer activity has not yet been confirmed in vivo, e.g., in suitable taste tests.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The “extra-cellular domain” and chemosensory receptor, e.g., T1R receptor regions or compositions described herein also include “analogs,” or “conservative variants” and “mimetics” (“peptidomimetics”) with structures and activity that substantially correspond to the exemplary sequences. Thus, the terms “conservative variant” or “analog” or “mimetic” refer to a polypeptide which has a modified amino acid sequence, such that the change(s) do not substantially alter the polypeptide's (the conservative variant's) structure and/or activity, as defined herein. These include conservatively modified variations of an amino acid sequence, i.e., amino acid substitutions, additions or deletions of those residues that are not critical for protein activity, or substitution of amino acids with residues having similar properties (e.g., acidic, basic, positively or negatively charged, polar or non-polar, etc.) such that the substitutions of even critical amino acids does not substantially alter structure and/or activity.

More particularly, “conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein.

For instance, the codons GCA, GCC, GCG and GCU all encode the amino acid alanine. Thus, at every position where an alanine is specified by a codon, the codon can be altered to any of the corresponding codons described without altering the encoded polypeptide.

Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

Conservative substitution tables providing functionally similar amino acids are well known in the art. For example, one exemplary guideline to select conservative substitutions includes (original residue followed by exemplary substitution): ala/gly or ser; arg/lys; asn/gln or his; asp/glu; cys/ser; gln/asn; gly/asp; gly/ala or pro; his/asn or gln; ile/leu or val; leu/ile or val; lys/arg or gln or glu; met/leu or tyr or ile; phe/met or leu or tyr; ser/thr; thr/ser; trp/tyr; tyr/trp or phe; val/ile or leu. An alternative exemplary guideline uses the following six groups, each containing amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (I); 5) Isoleucine (I), Leucine (L), Methionine (M), Valine (V); and 6) Phenylalanine (F), Tyrosine (Y), Tryptophan (W); (see also, e.g., Creighton, Proteins, W. H. Freeman and Company (1984); Schultz and Schimer, Principles of Protein Structure, Springer-Verlag (1979)). One of skill in the art will appreciate that the above-identified substitutions are not the only possible conservative substitutions. For example, for some purposes, one may regard all charged amino acids as conservative substitutions for each other whether they are positive or negative. In addition, individual substitutions, deletions or additions that alter, add or delete a single amino acid or a small percentage of amino acids in an encoded sequence can also be considered “conservatively modified variations.”

The terms “mimetic” and “peptidomimetic” refer to a synthetic chemical compound that has substantially the same structural and/or functional characteristics of the polypeptides, e.g., extra-cellular domain or any region therewith of T1R2 or T1R1. The mimetic can be either entirely composed of synthetic, non-natural analogs of amino acids, or may be a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity.

As with polypeptides of the invention which are conservative variants, routine experimentation will determine whether a mimetic is within the scope of the invention, i.e., that its structure and/or function is not substantially altered. Polypeptide mimetic compositions can contain any combination of non-natural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. A polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(O)—CH₂ for —C(O)—NH—), aminomethylene (—CH₂NH—), ethylene, olefin (—CH═CH—), ether (—CH₂O), thioether (CH₂—S—), tetrazole (—CN₄), thiazole, retroamide, thioamide, or ester (see, e.g., Spatola, Chemistry and Biochemistry of Amino Acids, Peptides and Proteins, Vol. 7, 267-357, Marcell Dekker, Peptide Backbone Modifications, NY (1983)). A polypeptide can also be characterized as a mimetic by containing all or some non-natural residues in place of naturally occurring amino acid residues; non-natural residues are well described in the scientific and patent literature.

“Compounds” refers to compounds encompassed by structural formulae disclosed herein and includes any specific compounds within these formulae whose structure is disclosed herein. Compounds may be identified either by their chemical structure and/or chemical name. When the chemical structure and chemical name conflict, the chemical structure is determinative of the identity of the compound. The compounds may also exist in several tautomeric forms including the enol form, the keto form and mixtures thereof. Accordingly, the chemical structures depicted herein encompass all possible tautomeric forms of the illustrated compounds. The compounds described also include isotopically labeled compounds where one or more atoms have an atomic mass different from the atomic mass conventionally found in nature. Examples of isotopes that may be incorporated into the compounds of the invention include, but are not limited to, ²H, ³H, ¹³C, ¹⁴C, ¹⁵N, ¹⁸O, ¹⁷O, etc. Compounds may exist in unsolvated forms as well as solvated forms, including hydrated forms and as N-oxides. In general, compounds may be hydrated, solvated or N-oxides. Certain compounds may exist in multiple crystalline or amorphous forms. In general, all physical forms are equivalent for the uses contemplated herein and are intended to be within the scope of the present invention. Further, it should be understood, when partial structures of the compounds are illustrated, that brackets indicate the point of attachment of the partial structure to the rest of the molecule.

“Alkyl,” by itself or as part of another substituent, refers to a saturated or unsaturated, branched, straight-chain or cyclic monovalent hydrocarbon radical derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl(allyl), cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like. The term “alkyl” is specifically intended to include groups having any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. Where a specific level of saturation is intended, the expressions “alkanyl,” “alkenyl,” and “alkynyl” are used. In some embodiments, an alkyl group comprises from 1 to 20 carbon atoms (C₁-C₂₀ alkyl). In other embodiments, an alkyl group comprises from 1 to 10 carbon atoms (C₁-C₁₀ alkyl). In still other embodiments, an alkyl group comprises from 1 to 6 carbon atoms (C₁-C₆ alkyl).

“Alkanyl,” by itself or as part of another substituent, refers to a saturated branched, straight-chain or cyclic alkyl radical derived by the removal of one hydrogen atom from a single carbon atom of a parent alkane. Typical alkanyl groups include, but are not limited to, methanyl; ethanyl; propanyls such as propan-1-yl, propan-2-yl(isopropyl), cyclopropan-1-yl, etc.; butanyls such as butan-1-yl, butan-2-yl(sec-butyl), 2-methyl-propan-1-yl(isobutyl), 2-methyl-propan-2-yl(t-butyl), cyclobutan-1-yl, etc.; and the like.

“Alkenyl,” by itself or as part of another substituent, refers to an unsaturated branched, straight-chain or cyclic alkyl radical having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl(allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Alkynyl,” by itself or as part of another substituent refers to an unsaturated branched, straight-chain or cyclic alkyl radical having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Alkoxy,” by itself or as part of another substituent, refers to a radical of the formula —O—R¹⁰⁰, where R¹⁰⁰ is alkyl or substituted alkyl as defined herein.

“Alkoxycarbonyl,” by itself or as part of another substituent, refers to a radical of the formula —C(O)—R¹⁰⁰, where R¹⁰⁰ is as defined above.

“Acyl” by itself or as part of another substituent refers to a radical —C(O)R¹⁰¹, where R¹⁰¹ is hydrogen, alkyl, substituted alkyl, aryl, substituted aryl, arylalkyl, substituted arylalkyl, heteroalkyl, substituted heteroalkyl, heteroarylalkyl or substituted heteroarylalkyl as defined herein. Representative examples include, but are not limited to formyl, acetyl, cyclohexylcarbonyl, cyclohexylmethylcarbonyl, benzoyl, benzylcarbonyl and the like.

“Aryl,” by itself or as part of another substituent, refers to a monovalent aromatic hydrocarbon group derived by the removal of one hydrogen atom from a single carbon atom of a parent aromatic ring system, as defined herein. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. In some embodiments, an aryl group comprises from 6 to 20 carbon atoms (C₆-C₂₀ aryl). In other embodiments, an aryl group comprises from 6 to 15 carbon atoms (C₆-C₁₅ aryl). In still other embodiments, an aryl group comprises from 6 to 15 carbon atoms (C₆-C₁₀ aryl).

“Arylalkyl,” by itself or as part of another substituent, refers to an acyclic alkyl group in which one of the hydrogen atoms bonded to a carbon atom, typically a terminal or sp³ carbon atom, is replaced with an aryl group as, as defined herein. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl and/or arylalkynyl is used. In some embodiments, an arylalkyl group is (C₆-C₃₀) arylalkyl, e.g., the alkanyl, alkenyl or alkynyl moiety of the arylalkyl group is (C₁-C₁₀) alkyl and the aryl moiety is (C₆-C₂₀) aryl. In other embodiments, an arylalkyl group is (C₆-C₂₀) arylalkyl, e.g., the alkanyl, alkenyl or alkynyl moiety of the arylalkyl group is (C₁-C₈) alkyl and the aryl moiety is (C₆-C₁₂) aryl. In still other embodiments, an arylalkyl group is (C₆-C₁₅) arylalkyl, e.g., the alkanyl, alkenyl or alkynyl moiety of the arylalkyl group is (C₁-C₅) alkyl and the aryl moiety is (C₆-C₁₀) aryl.

“Aryloxycarbonyl,” by itself or as part of another substituent, refers to a radical of the formula —C(O)—O—R¹⁰², where R¹⁰² is aryl, substituted aryl, arylalkyl, substituted arylalkyl, heteroaryl, substituted heteroaryl, heteroarylalkyl or substituted heteroarylalkyl.

“Cycloalkyl,” by itself or as part of another substituent, refers to a saturated or unsaturated cyclic alkyl radical, as defined herein. Where a specific level of saturation is intended, the nomenclature “cycloalkanyl” or “cycloalkenyl” is used. Typical cycloalkyl groups include, but are not limited to, groups derived from cyclopropane, cyclobutane, cyclopentane, cyclohexane, and the like. In some embodiments, the cycloalkyl group comprises from 3 to 10 ring atoms (C₃-C₁₀ cycloalkyl). In other embodiments, the cycloalkyl group comprises from 3 to 7 ring atoms (C₃-C₇ cycloalkyl).

“Cycloheteroalkyl,” by itself or as part of another substituent, refers to a saturated or unsaturated cyclic alkyl radical in which one or more carbon atoms (and optionally any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature “cycloheteroalkanyl” or “cycloheteroalkenyl” is used. Typical cycloheteroalkyl groups include, but are not limited to, groups derived from epoxides, azirines, thiiranes, imidazolidine, morpholine, piperazine, piperidine, pyrazolidine, pyrrolidone, quinuclidine, and the like. In some embodiments, the cycloheteroalkyl group comprises from 3 to 10 ring atoms (3-10 membered cycloheteroalkyl) In other embodiments, the cycloalkyl group comprise from 5 to 7 ring atoms (5-7 membered cycloheteroalkyl). A cycloheteroalkyl group may be substituted at a heteroatom, for example, a nitrogen atom, with a (C₁-C₆) alkyl group. As specific examples, N-methyl-imidazolidinyl, N-methyl-morpholinyl, N-methyl-piperazinyl, N-methyl-piperidinyl, N-methyl-pyrazolidinyl and N-methyl-pyrrolidinyl are included within the definition of “cycloheteroalkyl.” A cycloheteroalkyl group may be attached to the remainder of the molecule via a ring carbon atom or a ring heteroatom.

“Heteroalkyl,” “Heteroalkanyl,” “Heteroalkenyl” and “Heteroalkynyl,” “by themselves or as part of other substituents, refer to alkyl, alkanyl, alkenyl and alkynyl groups, respectively, in which one or more of the carbon atoms (and optionally any associated hydrogen atoms), are each, independently of one another, replaced with the same or different heteroatoms or heteroatomic groups. Typical heteroatoms or heteroatomic groups which can replace the carbon atoms include, but are not limited to, O, S, N, Si, —NH—, —S(O)—, —S(O)₂—, —S(O)NH—, —S(O)₂NH— and the like and combinations thereof. The heteroatoms or heteroatomic groups may be placed at any interior position of the alkyl, alkenyl or alkynyl groups. Typical heteroatomic groups which can be included in these groups include, but are not limited to, —O—, —S—, —O—O—, —S—S—, —O—S—, —NR¹⁰³R¹⁰⁴—, ═N—N═, —N═N—, —N═N—NR¹⁰⁵R¹⁰⁶, —PR¹⁰⁷—, —P(O)₂—, —POR¹⁰⁸—, —O—P(O)₂—, —SO—, —SO₂—, —SnR¹⁰⁹R¹¹⁰— and the like, where R¹⁰³, R¹⁰⁴, R¹⁰⁵, R¹⁰⁶, R¹⁰⁷, R¹⁰⁸, R¹⁰⁹ and R¹¹⁰ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl or substituted heteroarylalkyl.

“Heteroaryl,” by itself or as part of another substituent, refers to a monovalent heteroaromatic radical derived by the removal of one hydrogen atom from a single atom of a parent heteroaromatic ring systems, as defined herein. Typical heteroaryl groups include, but are not limited to, groups derived from acridine, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In some embodiments, the heteroaryl group comprises from 5 to 20 ring atoms (5-20 membered heteroaryl). In other embodiments, the heteroaryl group comprises from 5 to 10 ring atoms (5-10 membered heteroaryl). Exemplary heteroaryl groups include those derived from furan, thiophene, pyrrole, benzothiophene, benzofuran, benzimidazole, indole, pyridine, pyrazole, quinoline, imidazole, oxazole, isoxazole and pyrazine.

“Heteroarylalkyl” by itself or as part of another substituent refers to an acyclic alkyl group in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl and/or heteroarylalkynyl is used. In some embodiments, the heteroarylalkyl group is a 6-21 membered heteroarylalkyl, e.g., the alkanyl, alkenyl or alkynyl moiety of the heteroarylalkyl is (C₁-C₆) alkyl and the heteroaryl moiety is a 5-15-membered heteroaryl. In other embodiments, the heteroarylalkyl is a 6-13 membered heteroarylalkyl, e.g., the alkanyl, alkenyl or alkynyl moiety is (C₁-C₃) alkyl and the heteroaryl moiety is a 5-10 membered heteroaryl.

“Parent Aromatic Ring System” refers to an unsaturated cyclic or polycyclic ring system having a conjugated π electron system. Specifically included within the definition of “parent aromatic ring system” are fused ring systems in which one or more of the rings are aromatic and one or more of the rings are saturated or unsaturated, such as, for example, fluorene, indane, indene, phenalene, etc. Typical parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like.

“Parent Heteroaromatic Ring System” refers to a parent aromatic ring system in which one or more carbon atoms (and optionally any associated hydrogen atoms) are each independently replaced with the same or different heteroatom. Typical heteroatoms to replace the carbon atoms include, but are not limited to, N, P, O, S, Si, etc. Specifically included within the definition of “parent heteroaromatic ring system” are fused ring systems in which one or more of the rings are aromatic and one or more of the rings are saturated or unsaturated, such as, for example, benzodioxan, benzofuran, chromane, chromene, indole, indoline, xanthene, etc. Typical parent heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene and the like.

“Patient” includes humans. The terms “human” and “patient” are used interchangeably herein.

“Preventing” or “prevention” refers to a reduction in risk of acquiring a disease or disorder (i.e., causing at least one of the clinical symptoms of the disease not to develop in a patient that may be exposed to or predisposed to the disease but does not yet experience or display symptoms of the disease).

“Protecting group” refers to a grouping of atoms that when attached to a reactive functional group in a molecule masks, reduces or prevents reactivity of the functional group. Examples of protecting groups can be found in Green et al, “Protective Groups in Organic Chemistry”, (Wiley, 2^{nd} ed. 1991) and Harrison et al., “Compendium of Synthetic Organic Methods”, Vols. 1-8 (John Wiley and Sons, 1971-1996). Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”) and the like. Representative hydroxy protecting groups include, but are not limited to, those where the hydroxy group is either acylated or alkylated such as benzyl, and trityl ethers as well as alkyl ethers, tetrahydropyranyl ethers, trialkylsilyl ethers and allyl ethers.

“Salt” refers to a salt of a compound, which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

“Substituted,” when used to modify a specified group or radical, means that one or more hydrogen atoms of the specified group or radical are each, independently of one another, replaced with the same or different substituent(s). Substituent groups useful for substituting saturated carbon atoms in the specified group or radical include, but are not limited to —R^{a}, halo, —O⁻, ═O, —OR^{b}, —SR^{b}, —S⁻, ═S, —NR^{c}R^{c}, ═NR^{b}, ═N—OR^{b}, trihalomethyl, —CF₃, —CN, —OCN, —SCN, —NO, —NO₂, ═N₂, —N₃, —S(O)₂R^{b}, —S(O)₂NR^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O⁻), —P(O)(OR^{b})(OR^{b}), —C(O)R⁻, —C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(S)OR^{b}, —NR^{b}C(O)R^{b}, —NR^{b}C(S)R^{b}, —NR^{b}C(O)O⁻, —NR^{b}C(O)OR⁻, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a} is selected from the group consisting of alkyl, cycloalkyl, heteroalkyl, cycloheteroalkyl, aryl, arylalkyl, heteroaryl and heteroarylalkyl; each R^{b} is independently hydrogen or R^{a}; and each R^{c} is independently R^{b} or alternatively, the two R^{c}s are taken together with the nitrogen atom to which they are bonded form a 4-, 5-, 6- or 7-membered cycloheteroalkyl which may optionally include from 1 to 4 of the same or different additional heteroatoms selected from the group consisting of O, N and S. As specific examples, —NR^{c}R^{c} is meant to include —NH₂, —NH-alkyl, N-pyrrolidinyl and N-morpholinyl.

Similarly, substituent groups useful for substituting unsaturated carbon atoms in the specified group or radical include, but are not limited to, —R^{a}, halo, —O⁻, —OR^{b}, —SR^{b}, —S⁻, —NR^{c}R^{c}, trihalomethyl, —CF₃, —CN, —OCN, —SCN, —NO, —NO₂, —N₃, —S(O)₂R^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(S)OR^{b}, —NR^{b}C(O)R^{b}, —NR^{b}C(S)R^{b}, —NR^{b}C(O)O⁻, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a}, R^{b} and R^{c} are as previously defined.

Substituent groups useful for substituting nitrogen atoms in heteroalkyl and cycloheteroalkyl groups include, but are not limited to, —R^{a}, —O⁻, —OR^{b}, —SR^{b}, —S⁻, —NR^{c}R^{c}, trihalomethyl, —CF₃, —CN, —NO, —NO₂, —S(O)₂R^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)OR^{b}, —OC(S)OR^{b}, —NR^{b}C(O)R^{b}, —NR^{b}C(S)R^{b}, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a}, R^{b} and R^{c} are as previously defined.

Substituent groups from the above lists useful for substituting other specified groups or atoms will be apparent to those of skill in the art.

The substituents used to substitute a specified group can be further substituted, typically with one or more of the same or different groups selected from the various groups specified above.

“Treating” or “treatment” of any disease or disorder refers, in some embodiments, to ameliorating the disease or disorder (i.e., arresting or reducing the development of the disease or at least one of the clinical symptoms thereof). In other embodiments “treating” or “treatment” refers to ameliorating at least one physical parameter, which may not be discernible by the patient. In yet other embodiments, “treating” or “treatment” refers to inhibiting the disease or disorder, either physically, (e.g., stabilization of a discernible symptom), physiologically, (e.g., stabilization of a physical parameter) or both. In yet other embodiments, “treating” or “treatment” refers to delaying the onset of the disease or disorder.

“Therapeutically effective amount” means the amount of a compound that, when administered to a patient for treating a disease, is sufficient to effect such treatment for the disease. The “therapeutically effective amount” will vary depending on the compound, the disease and its severity and the age, weight, etc., of the patient to be treated.

“Vehicle” refers to a diluent, adjuvant, excipient or carrier with which a compound is administered.

The present invention is based, at least in part, on the discovery that an extra-cellular domain, e.g., the Venus flytrap domain of a chemosensory receptor, especially one or more interacting sites within the Venus flytrap domain, is a suitable target for compounds or other entities to modulate the chemosensory receptor and/or its ligands. Accordingly, the present invention provides screening methods for identifying chemosensory receptor modifiers as well as chemosensory receptor ligand modifiers. In addition, the present invention provides compounds and compositions capable of modulating chemosensory receptors as well as chemosensory receptor ligands.

According to one aspect of the present invention, it provides methods of screening for chemosensory receptor modifiers by determining whether a test entity is suitable to interact with a chemosensory receptor via one or more interacting sites within the extra-cellular domain of the chemosensory receptor, e.g., the Venus flytrap domain of the chemosensory receptor. According to another aspect of the present invention, it provides methods of screening for chemosensory receptor ligand modifiers by determining whether a test entity is suitable to interact with a chemosensory receptor, and optionally its ligand via one or more interacting sites within the extra-cellular domain, e.g., the Venus flytrap domain of the chemosensory receptor, optionally in the presence of a chemosensory receptor ligand.

In general, the extra-cellular domain of a chemosensory receptor refers to the extra-cellular amino-terminus of a chemosensory receptor and it usually includes a ligand-binding domain and a cysteine-rich linker domain, which connects the ligand-binding domain and the rest of the protein. In Class C GPCRs, the ligand binding domain is generally referred to as a Venus flytrap domain, the structure of which has been elucidated, e.g., using X-ray crystallography.

A Venus flytrap domain typically consists of two relatively rigid lobes connected by three strands forming a flexible “hinge” region. In the absence of a ligand, the Venus flytrap domain tends to adopt open conformations with well-separated lobes as well as closed conformations with lobes closer together. In one example, the Venus flytrap domain includes a region from amino acid 36 to amino acid 509 of human T1R1, amino acid 31 to amino acid 507 of human T1R2, and/or amino acid 35 to amino acid 511 of human T1R3.

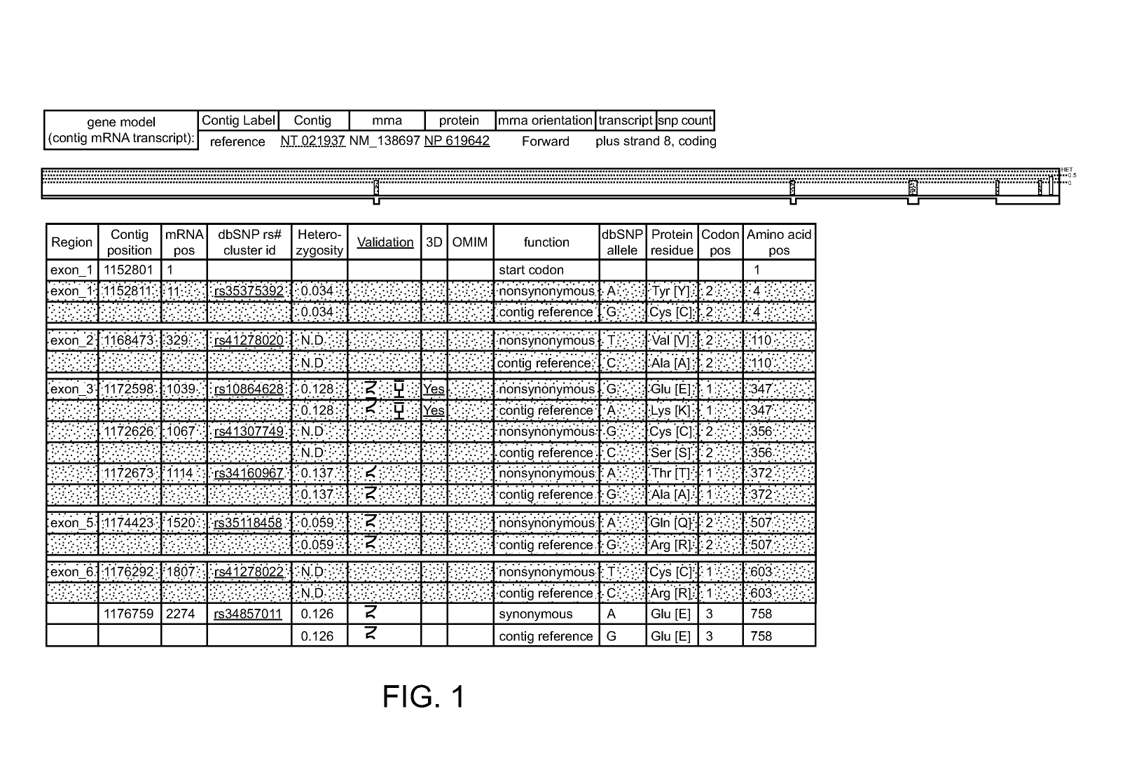

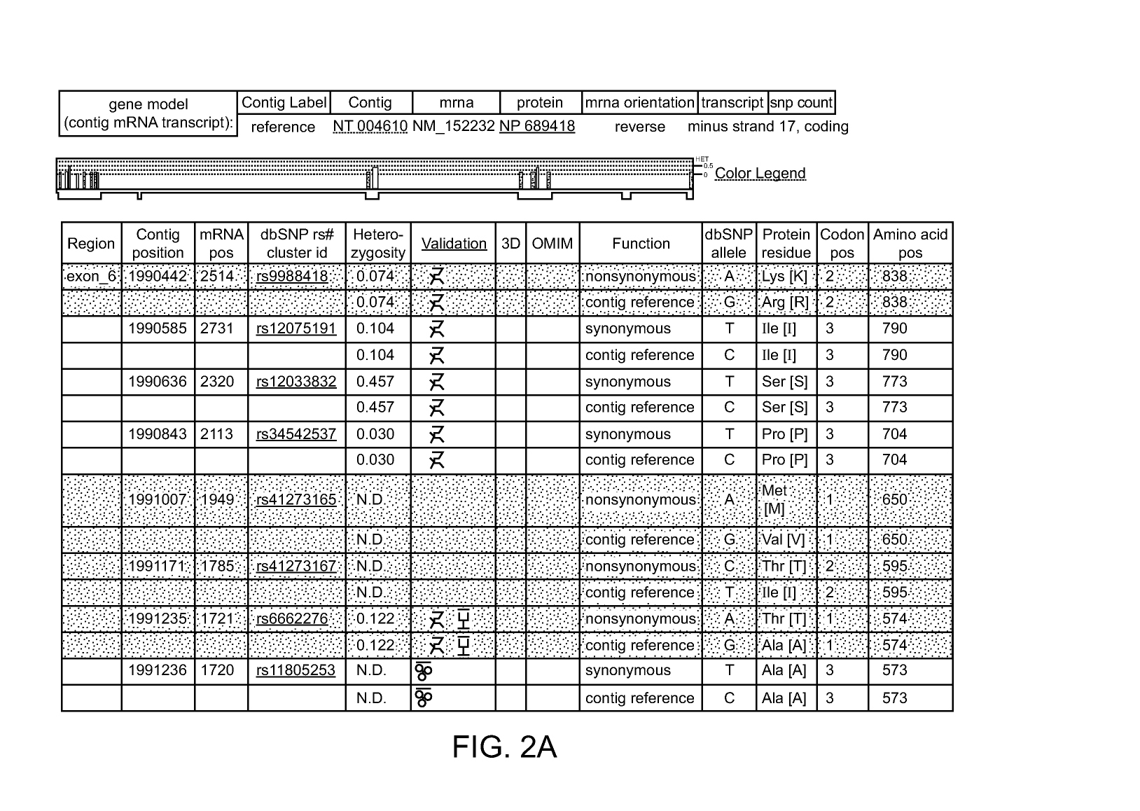

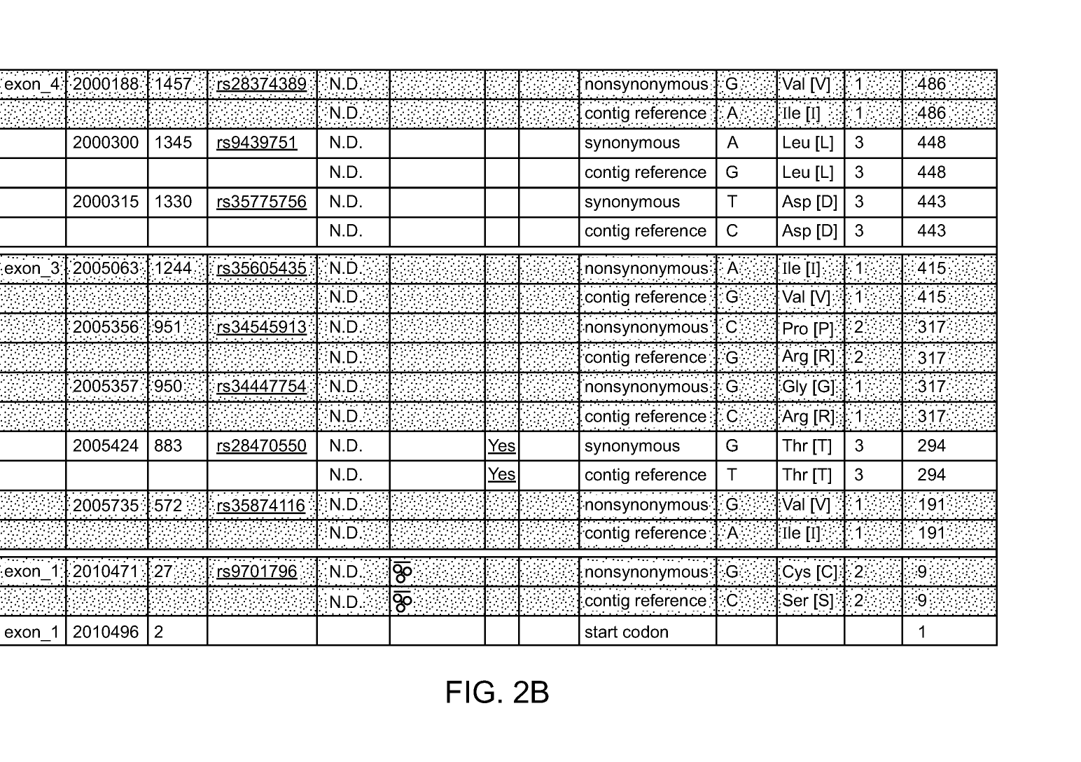

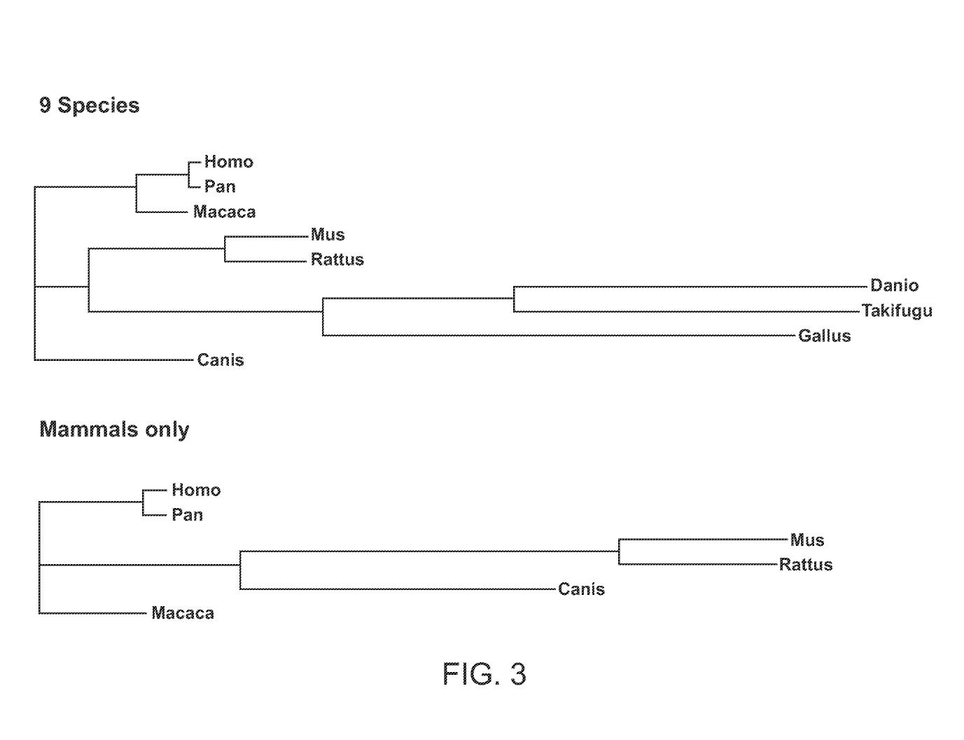

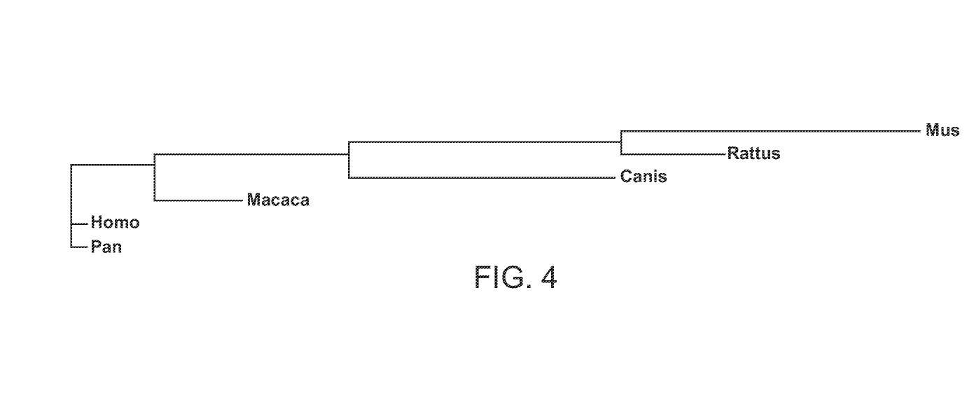

The Venus flytrap domain of the present invention includes any ligand binding domain or ligand interacting domain within the extra-cellular domain of a chemosensory receptor. In one embodiment, the Venus flytrap domain of the present invention includes any ligand binding domain of a member of the T1R family. In another embodiment, the Venus flytrap domain of the present invention includes any extra-cellular domain of a chemosensory receptor with a structure comprising two lobes connected by a hinge region. In yet another embodiment, the Venus flytrap domain of the present invention includes any domain corresponding to the structure and/or function of a region including amino acid 36 to amino acid 509 of human T1R1, amino acid 31 to amino acid 507 of human T1R2, and/or amino acid 35 to amino acid 511 of human T1R3. In still another embodiment, the Venus flytrap domain of the present invention includes any ligand binding domain of T1R1, T1R2, and/or T1R3 as well as any polymorphic variation, allele, or mutation thereof. Exemplary illustration of polymorphic variations for T1R1 and T1R2 is shown in FIGS. 1, 2, 3, and 4.

According to the present invention, a chemosensory receptor can be any receptor associated with chemosensory sensation or chemosensory ligand triggered signal transduction, e.g., via taste receptors or taste related receptors expressed in taste bud, gastrointestinal tract, etc. In one embodiment, a chemosensory receptor is a receptor that belongs to the 7-transmembrane receptor superfamily or G protein-coupled receptors (GPCRs). In another embodiment, a chemosensory receptor is a receptor carrying out signal transduction via one or more G proteins. In yet another embodiment, a chemosensory receptor is a receptor that belongs to family C or class C of GPCRs. In yet another embodiment, a chemosensory receptor is a receptor that belongs to the T1R family. In yet another embodiment, a chemosensory receptor is a receptor of T1R1, T1R2, T1R3, or their equivalences or variances or a combination thereof. In still another embodiment, a chemosensory receptor is a hetero-dimer of T1R1 and T1R3, or their equivalences or variances.

According to the present invention, an interacting site within the Venus flytrap domain of a chemosensory receptor can be one or more interacting residues or a three dimensional interacting space or a combination thereof. In one embodiment, the interacting site of the present invention is within the Venus flytrap domain of T1R1. In another embodiment, the interacting site of the present invention is within the Venus flytrap domain of T1R3. In yet another embodiment, the interacting site of the present invention is within the Venus flytrap domain of both T1R1 and T1R3.

Usually such an interacting site can be determined by any suitable means known or later discovered in the art. For example, such interacting site can be determined based on computer modeling, e.g., using software such as Homology or Modeller (by Accelrys Corporation) to construct three dimensional homology models of a chemosensory receptor Venus flytrap domain, e.g., the T1R1 and/or T1R3 Venus flytrap domains based on crystal structures of homologous Venus flytrap domains.

Such an interacting site can also be determined, e.g., based on X-ray crystallography and the three dimensional structure of a chemosensory receptor determined therefrom, e.g., the T1R1, T1R3, or T1R1/T1R3 heterodimer. Alternatively, for example, such an interacting site can be determined based on molecular mechanical techniques, e.g., normal mode analysis, loop generation techniques, Monte Carlo and/or molecular dynamics simulations to explore motions and alternative conformations of the Venus flytrap domains, docking simulations to dock candidate receptor ligands and candidate receptor ligand modifiers into these models or into experimentally determined structures of chemosensory receptors, e.g., T1R1 and T1R2.

In addition, for example, such an interacting sites can be determined based on mutagenesis, e.g., site-directed mutagenesis or a combination of two or more suitable methods known or later discovered, e.g., methods described herein.

In one example, such an interacting site is located in part of a chemosensory receptor, e.g., T1R1 and can be determined in the presence or absence of the other part of the chemosensory receptor, e.g., T1R3. In another example, such interacting site can be determined in the presence or absence of a chemosensory receptor modifier and/or chemosensory receptor ligand modifier.

In one embodiment, the interacting site within the Venus flytrap domain of a chemosensory receptor includes one or more interacting residues of the Venus flytrap domain of a chemosensory receptor. According to the present invention, the interacting residue of the Venus flytrap domain of a chemosensory receptor is a residue associated with any direct or indirect interaction between a chemosensory receptor and a chemosensory receptor modifier or a chemosensory receptor ligand modifier or both.

In one example, the interacting residue of the present invention includes any residue of a chemosensory receptor associated with an interaction between a chemosensory receptor modifier and a chemosensory receptor. In another example, the interacting residue of the present invention includes any residue of a chemosensory receptor associated with an interaction between a chemosensory receptor ligand modifier and a chemosensory receptor. In yet another example, the interacting residue of the present invention includes any residue of a chemosensory receptor associated with an interaction between a chemosensory receptor, a chemosensory receptor modifier and a chemosensory receptor ligand modifier.

In still another example, the interacting residue of the present invention includes any residue of a chemosensory receptor associated with an interaction between a chemosensory receptor and a umami flavor entity, e.g., any natural or synthesized umami flavor compounds including, without any limitation, L-amino acids (e.g., L-glutamate and L-aspartate), L-AP4 (2-amino-4-phosphonobutyrate), succinate, monosodium glutamate, etc.

In still another example, the interacting residue of the present invention includes any residue of a chemosensory receptor associated with an interaction between a chemosensory receptor and a umami flavor entity enhancer, e.g., inosine-5′-monophosphate (IMP), guanosine-5′-monophosphate (GMP), and compounds disclosed in International Publication Nos. WO 2006/084246 and WO 2006/084184, which are incorporated by reference in their entirety, etc. In still another example, the interacting residue of the present invention includes any residue of a chemosensory receptor associated with an interaction between a chemosensory receptor, a umami flavor entity, and a umami flavor entity enhancer.

In another instance, the interacting residue of the present invention is a residue within the Venus flytrap domain of a chemosensory receptor, wherein any mutation of which could result in a change of the activity of the chemosensory receptor or the impact of a chemosensory receptor ligand to the chemosensory receptor or both. For example, the interacting residue of the present invention can include any residue within the Venus flytrap domain of a chemosensory receptor, wherein the mutation of which results in a detectable change, e.g., qualitative or quantitative change of the activity of the chemosensory receptor in response to a chemosensory receptor modifier and/or chemosensory receptor ligand modifier.

In yet another instance, the interacting residue of the present invention is a residue within the Venus flytrap domain of a chemosensory receptor that interacts or forms productive interaction(s), e.g., van der Waals, burial of hydrophobic atoms or atomic groups, hydrogen bonds, ring stacking interactions, or salt-bridging electrostatic interactions with a chemosensory receptor modifier or chemosensory receptor ligand modifier, or both.

In still another instance, the interacting residue of the Venus flytrap domain of a chemosensory receptor can be any residue constituting one or more interacting structural components of the Venus flytrap domain, which are associated, directly or indirectly, with the interaction between a chemosensory receptor and a chemosensory receptor modifier or a chemosensory receptor ligand modifier or both. For example, the Venus flytrap domain structure of a chemosensory receptor generally includes two lobes joint by a hinge region. Residues constituting an interacting structural component of the Venus flytrap domain can be, e.g., residues constituting the hinge region, the inner side of each lobe, or residues on each lobe that are 1) positively charged and stabilizable by a chemosensory receptor ligand modifier, or 2) brought into close proximity during activation or conformational change of the Venus flytrap domain including without any limitation residues on the inner surfaces of the lobes pointing towards each other or on the tips of the lobes where the residues are partially exposed to solvent but still close to residues on the opposite lobe, etc. Examples of such residues include, without any limitation, H71, S385, S306, and E301 of a human T1R1 and H308, R281, H47, and R277 of a human T1R1.

Exemplary interacting residues of the Venus flytrap domain of a chemosensory receptor include any one or more residues or any group of residues of 1) D147, S148, T149, N150, A170, A171, S172, S173, D192, N195, D218, Y220, S276, R277, E301, and A302 of a human T1R1, 2) H47, S48, G49, C50, S67, F68, N69, E70, H71, S107, D147, S148, A170, F247, S276, R277, Q278, L279, A280, R281, V282, A302, W303, S306, R307, H308, I309, G311, R317, and W357 of a human T1R1, 3) L46, H47, S48, G49, C50, L51, S67, F68, N69, E70, H71, C106, S107, D108, D147, S148, R151, Y169, A170, Y220, F247, S248, S275, S276, R277, Q278, L279, A280, R281, V282, F283, F284, E285, E301, A302, W303, S306, R307, H308, I309, T310, G311, V312, P313, R317, K354, W357, K377, K379, M383, and S385 of a human T1R1, 4) L46, H47, S48, G49, C50, L51, S67, F68, N69, E70, H71, C106, S107, D108, D147, S148, T149, N150, R151, Y169, A170, A171, S172, S173, D192, N195, D218, Y220, S276, R277, E301, A302, F247, S248, S275, S276, R277, Q278, L279, A280, R281, V282, F283, F284, E285, E301, A302, W303, S306, R307, H308, I309, T310, G311, V312, P313, R317, K354, W357, K377, K379, M383, and S385 of a human T1R1, and 5) S172, Y220, D192, E301, and T149 of a human T1R1.

Exemplary interacting residues of the Venus flytrap domain of a chemosensory receptor with respect to a chemosensory receptor modifier include one or more residues of D147, S148, T149, N150, A170, A171, S172, S173, D192, N195, D218, Y220, S276, R277, E301, and A302 of a human T1R1.

Exemplary interacting residues of the Venus flytrap domain of a chemosensory receptor with respect to a umami flavor entity such as monosodium glutamate include one or more residues of D147, S148, T149, N150, A170, A171, S172, S173, D192, N195, D218, Y220, S276, R277, E301, and A302 of a human T1R1.

Exemplary interacting residues of the Venus flytrap domain of a chemosensory receptor with respect to a chemosensory receptor ligand modifier include one or more residues of L46, H47, S48, G49, C50, L51, S67, F68, N69, E70, H71, C106, S107, D108, D147, S148, R151, Y169, A170, Y220, F247, S248, S275, S276, R277, Q278, L279, A280, R281, V282, F283, F284, E285, E301, A302, W303, S306, R307, H308, I309, T310, G311, V312, P313, R317, K354, W357, K377, K379, M383, and S385 of a human T1R1.

Exemplary interacting residues of the Venus flytrap domain of a chemosensory receptor with respect to a chemosensory receptor ligand modifier, e.g., chemosensory receptor ligand enhancer include one or more residues of L46, H47, S48, G49, C50, L51, S67, F68, N69, E70, H71, C106, S107, D108, D147, S148, R151, Y169, A170, Y220, F247, S248, S275, S276, R277, Q278, L279, A280, R281, V282, F283, F284, E285, E301, A302, W303, S306, R307, H308, I309, T310, G311, V312, P313, R317, K354, W357, K377, K379, M383, and S385 of a human T1R1.

In the context of the present invention, any reference to a particular interacting residue, e.g., D147 of a human T1R1 receptor, includes all of its corresponding residues, e.g., 1) any residue of a human or non-human T1R1 that corresponds to the same position in any method of sequence alignment, 2) any residue of a human or non-human T1R1 that corresponds to the same position in any method of computer modeling in the presence or absence of a ligand or ligand modifier, 3) any residue of a human or non-human T1R1 that corresponds to the structural or functional role of the particular interacting residue, 4) any residue of a human or non-human T1R1 that is a polymorphic variation, alleles, mutation, etc. of the particular residue, 5) any residue of a human or non-human T1R1 that is a conservative substitution or conservatively modified variant of the particular residue, and 6) any corresponding residue of a human or non-human T1R1 in its modified form, e.g., artificial chemical mimetic of the particular interacting residue or un-modified form, e.g., naturally occurring form.

In another embodiment, the interacting site within the Venus flytrap domain of a chemosensory receptor is a three dimensional interacting space within the Venus flytrap domain outlined or defined, partially or entirely, by interacting residues or one or more interfaces, e.g., interacting points, lines or surfaces between a chemosensory receptor and one or more chemosensory receptor modifiers or chemosensory receptor ligand modifiers or a combination thereof. According to the present invention, a residue outlining or lining a space includes any residue having one or more backbones and/or side-chain atoms that are positioned so that they can potentially interact with atoms of a chemosensory receptor ligand or chemosensory receptor ligand modifier or both.

For example, the interacting space of the present invention can be any partial or whole space within the Venus flytrap domain that is usually occupied by one or more chemosensory receptor modifiers or chemosensory receptor ligand modifiers when they interact with a chemosensory receptor individually or together. In one example, the interacting space of the present invention is a space within the Venus flytrap domain usually occupied by a chemosensory receptor modifier, e.g., umami flavor entity. In another example, the interacting space of the present invention is a space within the Venus flytrap domain usually occupied by a chemosensory receptor ligand modifier, e.g., umami flavor enhancer in the presence of a chemosensory receptor ligand. In yet another example, the interacting space of the present invention is a space within the Venus flytrap domain usually occupied by a chemosensory receptor modifier, e.g., umami flavor entity and a chemosensory receptor ligand modifier, e.g., umami flavor entity enhancer. In still another example, the interacting space of the present invention is a space within the Venus flytrap domain that is defined, shaped, or transformed into based on an interaction between a chemosensory receptor and its ligand or its ligand modifier occurred partially or entirely outside of the space.

According to the present invention, the Venus flytrap domain of a chemosensory receptor can be generally viewed as two lobes joined by a hinge region. Exemplary interacting space within the Venus flytrap domain of a chemosensory receptor include any space associated with the hinge region, the inner side of one or two lobes, the tip of one or two lobes or a combination thereof of a chemosensory receptor.

Exemplary interacting space within the Venus flytrap domain of a chemosensory receptor with respect to a chemosensory receptor modifier includes any space within the Venus flytrap domain outlined or at least partially defined by the hinge region. According to the present invention, the hinge region usually comprises residues that are close to the three strands connecting the two lobes. In one example, the hinge region comprises residues that are homologous to residues observed coordinating agonists and antagonists in crystal structures of one or more Venus flytrap domains such as that of the mGluR receptor.

Exemplary interacting sites within the Venus flytrap domain of a chemosensory receptor with respect to a chemosensory receptor ligand modifier include any space outlined or at least partially defined by the inner side of one or two lobes away from the hinge region, as well as residues on the tips of the lobes that are brought into close proximity to residues on the other lobe.

In yet another embodiment, the interacting site within the Venus flytrap domain of a chemosensory receptor is a combination of one or more interacting residues with an interacting space of the chemosensory receptor. For example, the interacting site of a chemosensory receptor can be interacting residues associated with one interacting structural component of a chemosensory receptor in combination with a three dimensional space adjacent, e.g., not less than 1 Angstrom and not more than 30 Angstroms, to that interacting structural component. Another example of the interacting site of a chemosensory receptor includes interacting residues associated with one interacting structural component of a chemosensory receptor in combination with a three dimensional space apart from the interacting structural component.

In general, the screening methods provided by the present invention can be carried out by any suitable means known or later discovered. In one embodiment, the screening methods provided by the present invention are carried out in silico e.g., via “virtue screening” using any suitable computer modeling system or via specific or rational design of a compound using any suitable computer design system.

In another embodiment, the screening methods provided by the present invention are carried out via biological assays, e.g., high throughput screening of interactions between compounds and a chemosensory receptor or its fragments, e.g., genetically modified chemosensory receptors or fragments thereof such as mutated Venus flytrap domains of chemosensory receptors. In yet another embodiment, the screening methods provided by the present invention are carried out via a combination of biological assay(s) and computer modeling and/or design. For example, the screening methods provided by the present invention can be a combination of high-throughput screening of interactions between computer designed or pre-screened compounds and mutated Venus flytrap domains of chemosensory receptors.

In one example, the screening method provided by the present invention for chemosensory receptor modifiers includes determining an interacting site using a known chemosensory receptor modifier, e.g., structurally similar to a chemosensory receptor modifier of interest and then determining whether a test entity is suitable to interact with the chemosensory receptor via the interacting site so determined.

In another example, the screening method provided by the present invention for chemosensory receptor modifiers includes determining whether a test entity is suitable to interact with a chemosensory receptor via a predetermined interacting site, e.g., an interacting site selected or determined prior to screening, including without any limitation, selected or determined based on known chemosensory receptor modifiers or desired characteristics of a chemosensory receptor modifiers.

In yet another example, the screening method provided by the present invention for chemosensory receptor ligand modifiers includes determining a docking site for a chemosensory receptor ligand and subsequently determining whether a test entity is suitable to interact with the chemosensory receptor ligand via an interacting site selected in light of the docking of the chemosensory receptor ligand. According to the present invention, docking process can include any known or later discovered methods. For instance, docking can be a process in which the center of mass, orientations, and internal degrees of freedom of a molecule are modified to fit them into a predetermined space in a structural model. In one example, docking can be a process which includes translating and rotating a chemosensory receptor ligand relative to the chemosensory receptor structural model, e.g., Venus flytrap domain of a chemosensory receptor model while simultaneously adjusting internal torsional angles of the chemosensory receptor ligand to fit it into the interacting site of the chemosensory receptor. An example of a widely used docking program is GLIDE from Schroedinger, Inc.

In yet another example, the screening method provided by the present invention for chemosensory receptor ligand modifiers includes determining a docking site for a chemosensory receptor ligand and subsequently determining an interacting site using a known modifier of the chemosensory receptor ligand and then determining whether a test entity is suitable to interact with the chemosensory receptor ligand via the interacting site so determined.

In yet another example, the screening method provided by the present invention for chemosensory receptor ligand modifiers includes determining whether a test entity is suitable to interact with a chemosensory receptor via a predetermined interacting site for chemosensory receptor ligand modifiers.

In still another example, the screening method provided by the present invention for chemosensory receptor ligand modifiers includes determining whether a test entity is suitable to interact with a chemosensory receptor by determining, e.g., concurrently whether a chemosensory receptor ligand and the test entity are suitable to interact with the chemosensory receptor in a predetermined interacting site of the chemosensory receptor or an interacting site determined using known chemosensory receptor ligand and its modifier of interest.

In still another example, the screening method provided by the present invention for chemosensory receptor ligand modifiers includes determining whether a test entity is suitable to interact with a chemosensory receptor via an interacting site, either predetermined or not, as well as whether a test entity is suitable to interact with a chemosensory receptor ligand.

In still another example, the screening method provided by the present invention for chemosensory receptor ligand modifiers includes determining whether a test entity is suitable to interact with a chemosensory receptor via an interacting site, either pre-determined or not, as well as whether such interaction can stabilize a conformation, e.g., a semi-closed or closed conformation within the Venus flytrap domain formed by the interaction between a chemosensory receptor ligand and a chemosensory receptor, e.g., by forming productive additional interactions within the hinge region, lobes of the Venus flytrap domain, or tips of the flytrap domain via van der Waals, burial of hydrophobic atoms or atomic groups, hydrogen bonds, ring stacking interactions, or salt-bridging electrostatic interactions, etc.

In general, any suitable means known or later discovered can be used to determine whether a test entity is suitable to interact with an interacting site of the present invention. For example, one could determine the suitability of a test entity based on whether part or all of a test entity fits into a particular space entailed by an interacting site, e.g., whether a test entity fits into a particular space entailed by an interacting site substantially the same way a known chemosensory receptor modifier or chemosensory receptor ligand modifier does.

Alternatively, one could determine the suitability of a test entity with respect to an interacting site based on whether it forms interactions with a chemosensory receptor similar to the interactions formed by a known chemosensory receptor modifier or chemosensory receptor ligand modifier when they interact with the interacting site.

In addition, one could determine the suitability of a test entity based on whether it forms productive interactions with an interacting site, e.g., van der Waals, burial of hydrophobic atoms or atomic groups, hydrogen bonds, ring stacking interactions, or salt-bridging electrostatic interactions, etc. In one embodiment, one could determine the suitability of a test entity being a chemosensory receptor ligand modifier based on whether it forms productive interactions with an interacting site without forming van der Waals overlapping with one or more atoms of a chemosensory receptor or the chemosensory receptor ligand, e.g., in the context of one or more conformations of the Venus flytrap domain in light of the possible flexibility of the Venus flytrap domain.

According to the present invention, a test entity suitable to interact with one or more interacting sites within the Venus flytrap domain of a chemosensory receptor is indicative of a candidate for a chemosensory receptor modifier or chemosensory receptor ligand modifier. In one embodiment, a test entity suitable to interact with one or more interacting sites within the Venus flytrap domain of T1R1 is indicative of a candidate for a T1R1 receptor modifier or T1R1 receptor ligand modifier. In another embodiment, a test entity suitable to interact with one or more interacting sites within the Venus flytrap domain of T1R1 is indicative of a candidate for a T1R receptor modifier or T1R receptor ligand modifier. In yet another embodiment, a test entity suitable to interact with one or more interacting sites within the Venus flytrap domain of T1R1 is indicative of a candidate for a receptor modifier or receptor ligand modifier for a receptor of GPCR superfamily. In still another embodiment, a test entity suitable to interact with one or more interaction sites within the Venus flytrap domain of a chemosensory receptor is indicative of a candidate for a receptor modifier or receptor ligand modifier of a receptor that corresponds to the chemosensory receptor or belongs to the same family or class as of the chemosensory receptor.

According to the present invention, a test entity can be any compound or molecule, e.g., any compound or entity that potentially could be a source for a desired chemosensory receptor modifier or chemosensory receptor ligand modifier. For example, a test entity can be a member of a combinatorial library, a member of a natural compound library, a “specifically designed” compound that is designed based on various desirable features or rationales, etc.

In general, a chemosensory receptor modifier or ligand includes any compound or entity capable of interacting with, e.g., binding to a chemosensory receptor or modulating the structure or function of a chemosensory receptor, e.g., activate, deactivate, increase, or decrease the signal transduction activity of a chemosensory receptor, especially via G-protein signal transduction pathway.

In one embodiment, a chemosensory receptor modifier or ligand is a compound or entity with umami flavor including without any limitation any natural or synthesized umami flavor compound including, without any limitation, L-amino acids, L-AP4, succinate, monosodium glutamate, etc.

In another embodiment, a chemosensory receptor modifier or ligand is a compound or entity capable of activating a chemosensory receptor, e.g., activating the G-protein signal transduction pathway associated with the chemosensory receptor. In yet another embodiment, a chemosensory receptor modifier or ligand is a compound or entity capable of blocking or decreasing the activation of a chemosensory receptor. In still another embodiment, a chemosensory receptor modifier or ligand is a compound or entity capable of modulating the activity of a chemosensory receptor and inducing a therapeutically desirable reaction or signal transduction. In still another embodiment, a chemosensory receptor modifier or ligand is a chemosensory receptor ligand modifier.

According to the present invention, a chemosensory receptor ligand modifier includes any compound or entity capable of interacting or modulating the activity of a chemosensory receptor modifier or the activity of a chemosensory receptor in the presence of a chemosensory receptor modifier. In one embodiment, a chemosensory receptor ligand modifier is an enhancer of a chemosensory receptor modifier. In another embodiment, a chemosensory receptor ligand modifier is an antagonist of a chemosensory receptor modifier. In yet another embodiment, a chemosensory receptor ligand modifier is an enhancer of a chemosensory receptor modifier without having substantial activity of the chemosensory receptor modifier. In still another embodiment, a chemosensory receptor ligand modifier is an enhancer of a umami flavored compound without having substantial umami flavor by itself, e.g., as judged by animals or humans such as majority of a panel of at least eight human taste testers, via procedures commonly known in the field.

According to another aspect of the present invention, it provides chemosensory receptor ligand modifiers. In one embodiment, it provides chemosensory receptor ligand modifiers identified by the screen methods of the present invention. In another embodiment, it provides chemosensory receptor ligand modifiers capable of interacting with a chemosensory receptor via an interacting site of the present invention. In yet another embodiment, it provides chemosensory receptor ligand modifiers capable of interacting with a chemosensory receptor via one or more interacting residues of the chemosensory receptor. In still another embodiment, it provides chemosensory receptor ligand modifiers capable of interacting with a chemosensory receptor via an interacting space within the Venus flytrap domain that is outlined, defined, or shaped, partially or entirely, by interacting residues of the chemosensory receptor. In still yet another embodiment, it provides chemosensory receptor ligand modifiers excluding, e.g., known natural or synthesized umami enhancers such as IMP, GMP, AMP, etc.

In the context of the present invention, “capable of interacting with” or “interacting with” means that a compound or molecule binds to or forms one or more molecular interactions, e.g., productive interactions with another molecule, e.g., a chemosensory receptor. Exemplary molecular interactions, e.g., productive interactions include van der Waals, burial of hydrophobic atoms or atomic groups, hydrogen bonds, ring stacking interactions, salt-bridging electrostatic interactions, or a combination thereof.

In one embodiment, the present invention provides chemosensory receptor ligand modifiers capable of interacting with a chemosensory receptor via a group of interacting residues or a space within the Venus flytrap domain that is outlined, shaped, or defined, partially or entirely by the group or any subgroup of interacting residues, optionally in the presence of a chemosensory receptor ligand, e.g., umami flavor entity. Exemplary groups of interacting residues include, without any limitation, 1) D147, S148, T149, N150, A170, A171, S172, S173, D192, N195, D218, Y220, S276, R277, E301, and A302 of a human T1R1, 2) H47, S48, G49, C50, S67, F68, N69, E70, H71, S107, D147, S148, A170, F247, S276, R277, Q278, L279, A280, R281, V282, A302, W303, S306, R307, H308, I309, G311, R317, and W357 of a human T1R1, 3) L46, H47, S48, G49, C50, L51, S67, F68, N69, E70, H71, C106, S107, D108, D147, S148, R151, Y169, A170, Y220, F247, S248, S275, S276, R277, Q278, L279, A280, R281, V282, F283, F284, E285, E301, A302, W303, S306, R307, H308, I309, T310, G311, V312, P313, R317, K354, W357, K377, K379, M383, and S385 of a human T1R1, 4) L46, H47, S48, G49, C50, L51, S67, F68, N69, E70, H71, C106, S107, D108, D147, S148, T149, N150, R151, Y169, A170, A171, S172, S173, D192, N195, D218, Y220, S276, R277, E301, A302, F247, S248, S275, S276, R277, Q278, L279, A280, R281, V282, F283, F284, E285, E301, A302, W303, S306, R307, H308, I309, T310, G311, V312, P313, R317, K354, W357, K377, K379, M383, and S385 of a human T1R1, 5) S172, Y220, D192, E301, and T149 of a human T1R1, and 6) a combination thereof.

In another embodiment, the present invention provides chemosensory receptor ligand enhancers capable of interacting with a chemosensory receptor in the presence of a chemosensory receptor ligand via one or more interacting residues of L46, H47, S48, G49, C50, L51, S67, F68, N69, E70, H71, C106, S107, D108, D147, S148, R151, Y169, A170, Y220, F247, S248, S275, S276, R277, Q278, L279, A280, R281, V282, F283, F284, E285, E301, A302, W303, S306, R307, H308, I309, T310, G311, V312, P313, R317, K354, W357, K377, K379, M383, and S385 of a human T1R1.

In yet another embodiment, the present invention provides chemosensory receptor ligand enhancers capable of interacting with a chemosensory receptor in the presence of a umami flavor entity via one or more interacting residues of L46, H47, S48, G49, C50, L51, S67, F68, N69, E70, H71, C106, S107, D108, D147, S148, R151, Y169, A170, Y220, F247, S248, S275, S276, R277, Q278, L279, A280, R281, V282, F283, F284, E285, E301, A302, W303, S306, R307, H308, I309, T310, G311, V312, P313, R317, K354, W357, K377, K379, M383, and S385 of a human T1R1.

In still another embodiment, the present invention provides chemosensory receptor ligand modifiers capable of interacting with a chemosensory receptor, optionally in the presence of a chemosensory receptor ligand via at least 1, 2, 3, 4, 5, 6, 7, 8, 9, or 10 interacting residues selected from the group of L46, H47, S48, G49, C50, L51, S67, F68, N69, E70, H71, C106, S107, D108, D147, S148, T149, N150, R151, Y169, A170, A171, S172, S173, D192, N195, D218, Y220, S276, R277, E301, A302, F247, S248, S275, S276, R277, Q278, L279, A280, R281, V282, F283, F284, E285, E301, A302, W303, S306, R307, H308, I309, T310, G311, V312, P313, R317, K354, W357, K377, K379, M383, and S385 of a human T1R1.

In still yet another embodiment, the present invention provides chemosensory receptor ligand modifiers capable of interacting with a chemosensory receptor to stabilize a conformation, e.g., semi-closed or closed conformation formed by the interaction between a chemosensory receptor and a chemosensory receptor ligand. In one example, chemosensory receptor ligand modifiers of the present invention are charged, e.g., positively charged so that they are capable of stabilizing a group of oppositely charged, e.g., negatively charged residues on one or both lobes of a chemosensory receptor.

According to yet another aspect of the present invention, it provides chemosensory receptor modifiers. In one embodiment, it provides chemosensory receptor modifiers identified by the screen methods of the present invention. In another embodiment, it provides chemosensory receptor modifiers capable of interacting with a chemosensory receptor via an interacting site of the present invention. In yet another embodiment, it provides chemosensory receptor modifiers capable of interacting with a chemosensory receptor via one or more interacting residues of the chemosensory receptor. In still another embodiment, it provides chemosensory receptor modifiers capable of interacting with a chemosensory receptor via an interacting space within the Venus flytrap domain that is outlined, defined, or shaped, partially or entirely, by interacting residues of the chemosensory receptor. In still yet another embodiment, it provides chemosensory receptor modifiers excluding, e.g., known natural or synthesized umami flavor entities such as L-glutamate, L-aspartate, succinate, monosodium glutamate, etc.

In one embodiment, the present invention provides chemosensory receptor modifiers capable of interacting with a chemosensory receptor via a group of interacting residues or a space within the Venus flytrap domain that is outlined, shaped, or defined, partially or entirely by the group or any subgroup of interacting residues, e.g., 1) D147, S148, T149, N150, A170, A171, S172, S173, D192, N195, D218, Y220, S276, R277, E301, and A302 of a human T1R1, 2) L46, H47, S48, G49, C50, L51, S67, F68, N69, E70, H71, C106, S107, D108, D147, S148, T149, N150, R151, Y169, A170, A171, S172, S173, D192, N195, D218, Y220, S276, R277, E301, A302, F247, S248, S275, S276, R277, Q278, L279, A280, R281, V282, F283, F284, E285, E301, A302, W303, S306, R307, H308, I309, T310, G311, V312, P313, R317, K354, W357, K377, K379, M383, and S385 of a human T1R1, and 3) a combination thereof.

In another embodiment, the present invention provides chemosensory receptor modifiers, e.g., activators capable of interacting with a chemosensory receptor via at least 1, 2, 3, 4, 5, 6, 7, 8, 9, or 10 interacting residues selected from the group of L46, H47, S48, G49, C50, L51, S67, F68, N69, E70, H71, C106, S107, D108, D147, S148, T149, N150, R151, Y169, A170, A171, S172, S173, D192, N195, D218, Y220, S276, R277, E301, A302, F247, S248, S275, S276, R277, Q278, L279, A280, R281, V282, F283, F284, E285, E301, A302, W303, S306, R307, H308, I309, T310, G311, V312, P313, R317, K354, W357, K377, K379, M383, and S385 of a human T1R1 of a human T1R1.

According to still another aspect of the present invention, it provides methods for modulating a chemosensory receptor and/or its ligand by modulating one or more interacting sites of the chemosensory receptor. For example, one can modulate a chemosensory receptor by contacting, in vivo or in vitro, a chemosensory receptor modifier or chemosensory receptor ligand modifier or both, (e.g., optionally excluding umami enhancers or umami flavor entities known prior to the present invention) with cells containing the chemosensory receptor, wherein the chemosensory receptor modifier or chemosensory receptor ligand is capable of interacting with or targeting one or more interacting sites of the chemosensory receptor.

In one embodiment, the method of modulating a chemosensory receptor and/or its ligand is by modulating one or more interacting residues or interacting spaces or a combination thereof. In another embodiment, the method of modulating a chemosensory receptor and/or its ligand is by interacting with one or more interacting residues in the presence of a chemosensory receptor ligand. In yet another embodiment, the method of modulating a chemosensory receptor or its ligand includes modulating the impact of a chemosensory receptor ligand on the chemosensory receptor by interacting with the chemosensory receptor via one or more interacting residues in the presence of the chemosensory receptor ligand.

In yet another embodiment, the method of modulating a chemosensory receptor and/or its ligand is by interacting with the chemosensory receptor via a group of interacting residues or a space outlined, shaped, or defined, partially or entirely, by the group or subgroup of interacting residues, optionally in the presence of a chemosensory receptor ligand, e.g., umami flavor entity. Exemplary groups of such interacting residues include, without any limitation, 1) D147, S148, T149, N150, A170, A171, S172, S173, D192, N195, D218, Y220, S276, R277, E301, and A302 of a human T1R1, 2) H47, S48, G49, C50, S67, F68, N69, E70, H71, S107, D147, S148, A170, F247, S276, R277, Q278, L279, A280, R281, V282, A302, W303, S306, R307, H308, I309, G311, R317, and W357 of a human T1R1, 3) L46, H47, S48, G49, C50, L51, S67, F68, N69, E70, H71, C106, S107, D108, D147, S148, R151, Y169, A170, Y220, F247, S248, S275, S276, R277, Q278, L279, A280, R281, V282, F283, F284, E285, E301, A302, W303, S306, R307, H308, I309, T310, G311, V312, P313, R317, K354, W357, K377, K379, M383, and S385 of a human T1R1, 4) L46, H47, S48, G49, C50, L51, S67, F68, N69, E70, H71, C106, S107, D108, D147, S148, T149, N150, R151, Y169, A170, A171, S172, S173, D192, N195, D218, Y220, S276, R277, E301, A302, F247, S248, S275, S276, R277, Q278, L279, A280, R281, V282, F283, F284, E285, E301, A302, W303, S306, R307, H308, I309, T310, G311, V312, P313, R317, K354, W357, K377, K379, M383, and S385 of a human T1R1, 5) S172, Y220, D192, E301, and T149 of a human T1R1, and 6) a combination thereof.

In yet another embodiment, the method of modulating a chemosensory receptor and/or its ligand is by interacting with the chemosensory receptor via one or more interacting residues of D147, S148, T149, N150, A170, A171, S172, S173, D192, N195, D218, Y220, S276, R277, E301, and A302 of a human T1R1.

In yet another embodiment, the method of modulating a chemosensory receptor and/or its ligand is by interacting with the chemosensory receptor, optionally in the presence of a chemosensory receptor ligand via one or more interacting residues of L46, H47, S48, G49, C50, L51, S67, F68, N69, E70, H71, C106, S107, D108, D147, S148, R151, Y169, A170, Y220, F247, S248, S275, S276, R277, Q278, L279, A280, R281, V282, F283, F284, E285, E301, A302, W303, S306, R307, H308, I309, T310, G311, V312, P313, R317, K354, W357, K377, K379, M383, and S385 of a human T1R1.

In still another embodiment, the method of enhancing a chemosensory receptor and/or its ligand is by interacting with the chemosensory receptor, optionally in the presence of a chemosensory receptor ligand via one or more interacting residues of L46, H47, S48, G49, C50, L51, S67, F68, N69, E70, H71, C106, S107, D108, D147, S148, R151, Y169, A170, Y220, F247, S248, S275, S276, R277, Q278, L279, A280, R281, V282, F283, F284, E285, E301, A302, W303, S306, R307, H308, I309, T310, G311, V312, P313, R317, K354, W357, K377, K379, M383, and S385 of a human T1R1.

In still another embodiment, the method of modulating a chemosensory receptor and/or its ligand is by interacting with the chemosensory receptor, optionally in the presence of a chemosensory receptor ligand via at least 1, 2, 3, 4, 5, 6, 7, 8, 9, or 10 interacting residues selected from the group of L46, H47, S48, G49, C50, L51, S67, F68, N69, E70, H71, C106, S107, D108, D147, S148, T149, N150, R151, Y169, A170, A171, S172, S173, D192, N195, D218, Y220, S276, R277, E301, A302, F247, S248, S275, S276, R277, Q278, L279, A280, R281, V282, F283, F284, E285, E301, A302, W303, S306, R307, H308, I309, T310, G311, V312, P313, R317, K354, W357, K377, K379, M383, and S385 of a human T1R1.

According to the present invention, a method of modulating a chemosensory receptor and/or its ligand includes modulating the activity, structure, function, expression, and/or modification of a chemosensory receptor as well as modulating, treating, or taking prophylactic measure of a condition, e.g., physiological or pathological condition, associated with a chemosensory receptor.

In general, a physiological or pathological condition associated with a chemosensory receptor includes a condition associated with a taste, e.g., sweet, umami, bitter, sour, salty, or a combination thereof or a condition associated with, e.g., gastrointestinal system, metabolic disorders, functional gastrointestinal disorders, etc.

In one embodiment, the method of the present invention, e.g., modulating a chemosensory receptor and/or its ligand includes modulating, increasing or decreasing a sweet or umami taste or a subject's reaction, physiological or otherwise, to a sweet or umami taste. In another embodiment, the method of the present invention, e.g., modulating a chemosensory receptor and/or its ligand includes enhancing a sweet or umami taste or a subject's reaction, physiological or otherwise, to a sweet or umami taste.

In yet another embodiment, the method of the present invention, e.g., modulating a chemosensory receptor and/or its ligand includes modulation, treatment, and/or prophylactic measure of a condition associated with gastrointestinal system including without any limitation conditions associated with esophageal motility (e.g., cricopharyngeal achalasia, globus hystericus, achalasia, diffuse esophageal spasm and related motor disorders, scleroderma involving the esophagus, etc.), inflammatory disorders (e.g., gastroesophageal reflux and esophagitis, infectious esophagitis, etc.), peptic ulcer, duodenal ulcer, gastric ulcer, gastrinoma, stress ulcers and erosions, drug-associated ulcers and erosions, gastritis, esophageal cancer, tumors of the stomach, disorders of absorption (e.g., absorption of specific nutrients such as carbohydrate, protein, amino acid, fat, cholesterol and fat-soluble vitamins, water and sodium, calcium, iron, water-soluble vitamins, etc.), disorders of malabsorption, defects in mucosal function (e.g., inflammatory or infiltrative disorders, biochemical or genetic abnormalities, endocrine and metabolic disorders, protein-losing enteropathy, etc.), autoimmune diseases of the digestive tract (e.g., celiac disease, Crohn's disease, ulcerative colitis, etc.), irritable bowel syndrome, inflammatory bowel disease, complications of inflammatory bowel disease, extraintestinal manifestations of inflammatory bowel disease, disorders of intestinal motility, vascular disorders of the intestine, anorectial disorders (e.g., hemorrhoids, anal inflammation, etc.), colorectal cancer, tumors of the small intestine, cancers of the anus, derangements of hepatic metabolism, hyperbilirubinemia, hepatitis, alcoholic liver disease and cirrhosis, biliary cirrhosis, neoplasms of the liver, infiltrative and metabolic diseases affecting the liver (e.g., fatty liver, reye's syndrome, diabetic glycogenosis, glycogen storage disease, Wilson's disease, hemochromatosis), diseases of the gallbladder and bile ducts, disorders of the pancreas (e.g., pancreatitis, pancreatic exocrine insufficiency, pancreatic cancer, etc.), endocrine tumors of the gastrointestinal tract and pancreas.

In still another embodiment, the method of the present invention, e.g., modulating a chemosensory receptor and/or its ligand includes modulation, treatment, and/or prophylactic measure of a condition associated with metabolic disorders, e.g., appetite, body weight, food or liquid intake or a subject's reaction to food or liquid intake, or state of satiety or a subject's perception of a state of satiety, nutrition intake and regulation, (e.g., protein-energy malnutrition, physiologic impairments associated with protein-energy malnutrition, etc.), obesity, secondary obesity (e.g., hypothyroidism, Cushing's disease, insullinoma, hypothalamic disorders, etc.), eating disorders (e.g., anorexia nervosa, bulimia, etc.), vitamin deficiency and excess, insulin metabolism, diabetes (type I and type II) and complications thereof (e.g., circulatory abnormalities, retinopathy, diabetic nephropathy, diabetic neuropathy, diabetic foot ulcers, etc.), glucose metabolism, fat metabolism, hypoglycemia, hyperglycermia, hyperlipoproteinemias, etc.

In still yet another embodiment, the method of the present invention, e.g., modulating a chemosensory receptor and/or its ligand includes modulation, treatment, and/or prophylactic measure of a condition associated with functional gastrointestinal disorders, e.g., in the absence of any particular pathological condition such as peptic ulcer and cancer, a subject has abdominal dyspepsia, e.g., feeling of abdominal distention, nausea, vomiting, abdominal pain, anorexia, reflux of gastric acid, or abnormal bowel movement (constipation, diarrhea and the like), optionally based on the retention of contents in gastrointestinal tract, especially in stomach. In one example, functional gastrointestinal disorders include a condition without any organic disease of the gastrointestinal tract, but with one or more reproducible gastrointestinal symptoms that affect the quality of life of a subject, e.g., human.

Exemplary functional gastrointestinal disorders include, without any limitation, functional dyspepsia, gastroesophageal reflux condition, diabetic gastroparesis, reflux esophagitis, postoperative gastrointestinal dysfunction and the like, nausea, vomiting, sickly feeling, heartburn, feeling of abdominal distention, heavy stomach, belching, chest writhing, chest pain, gastric discomfort, anorexia, dysphagia, reflux of gastric acid, abdominal pain, constipation, diarrhea, breathlessness, feeling of smothering, low incentive or energy level, pharyngeal obstruction, feeling of foreign substance, easy fatigability, stiff neck, myotonia, mouth dryness (dry mouth, thirst, etc.) tachypnea, burning sensation in the gastrointestinal tract, cold sensation of extremities, difficulty in concentration, impatience, sleep disorder, headache, general malaise, palpitation, night sweat, anxiety, dizziness, vertigo, hot flash, excess sweating, depression, etc.

In still yet another embodiment, the method of the present invention, e.g., modulating a chemosensory receptor and/or its ligand includes increasing or promoting digestion, absorption, blood nutrient level, and/or motility of gastrointestinal tract in a subject, e.g., promotion of gastric emptying (e.g., clearance of stomach contents), reduction of abdominal distention in the early postprandial period, improvement of anorexia, etc. In general, such promotion can be achieved either directly or via increasing the secretion of a regulatory entity, e.g., hormones, etc.

In still yet another embodiment, the method of the present invention, e.g., modulating a chemosensory receptor and/or its ligand includes increasing one or more gastrointestinal functions of a subject, e.g., to improve the quality of life or healthy state of a subject.

In still yet another embodiment, the method of the present invention, e.g., modulating a chemosensory receptor and/or its ligand includes modulating the activity of T1R (e.g., T1R1, T1R2, or T1R3) expressing cells, e.g., liver cells (e.g., hepatocytes, endothelial cells, Kupffer cells, Stellate cells, epithelial cells of bile duct, etc.), heart cells (e.g., endothelial, cardiac, and smooth muscle cells, etc.), pancreatic cells (e.g., alpha cell, beta cell, delta cell, neurosecretory PP cell, D1 cell, etc.), cells in the nipple (e.g., ductal epithelial cells, etc.), stomach cells (e.g., mucous cells, parietal cells, chief cells, G cells, P/D1 cells), intestinal cells (e.g., enteroendocrine cells, brush cells, etc.), salivary gland cells (e.g., Seromucous cells, mucous cells, myoepithelial cells, intercalated duct cell, striated duct cell, etc.), L cells (e.g. expressing GLP-1, etc.), enterochromaffin cells (e.g., expressing serotonin), enterochromaffin-like cells, G cells (e.g., expressing gastrin), D cells (delta cells, e.g., expressing somatostatin), I cells (e.g., expressing cholescystokinin (CCK), K cells (e.g., expressing gastric inhibitory polypeptide), P/D1 cells (e.g., expressing ghrelin), chief cells (e.g., expressing pepsin), and S cells (e.g., expressing secretin). In one example, the method of the present invention includes increasing the expression level of T1R in T1R expressing cells. In another example, the method of the present invention includes increasing the secretion level of T1R expressing cells.

In still yet another embodiment, the method of the present invention, e.g., modulating a chemosensory receptor and/or its ligand includes modulating the expression, secretion, and/or functional level of T1R expressing cells associated with hormone, peptide, enzyme producing. In one example, the method of the present invention includes modulating the level of glucose, e.g., inhibitors of a chemosensory receptor such as T1R2 can be used to decrease glucose level (e.g., glucose absorption) in a subject. In another example, the method of the present invention includes modulating the level of incretins, e.g., agonist of a chemosensory receptor such as T1R2 can be used to increase glucagons-like peptide 1 (GLP-1) and thus increase the production of insulin. In yet another example, the method of the present invention includes modulating the expression, secretion, and/or activity level of hormones or peptides produced by T1R expressing cells or gastrointestinal hormone producing cells, e.g., ligands for 5HT receptors (e.g., serotonin), incretins (e.g., GLP-1 and glucose-dependent insulinotropic polypeptide (GIP)), gastrin, secretin, pepsin, cholecystokinin, amylase, ghrelin, leptin, somatostatin, etc. In still another example, the method of the present invention includes modulating the pathways associated with hormones, peptides, and/or enzymes secreted by T1R expressing cells.

Exemplary chemosensory receptor ligand modifiers provided by the present invention and/or suitable to be used for the methods of the present invention include compounds of the following formulae.

In a first aspect, a compound of structural Formula (I) is provided:

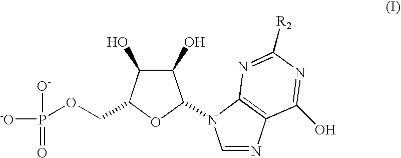

or a salt, hydrate or solvate thereof, wherein:

R₂ is hydrogen, —NR₄R₅ or —NR₄C(O)R₅;

R₄ and R₅ are independently hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl or substituted heteroarylalkyl;

R₃ is hydroxyl, —NR₆R₇, —NR₆C(O)R₇ or —S(O)ₐR₆;

R₆ and R₇ are independently hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl or substituted heteroarylalkyl; and

a is 0, 1 or 2;

provided that when R₂ is hydrogen then R₃ is not hydroxyl; and

when R₂ is —NH₂ then R₃ is not hydroxyl.

In some embodiments, when R₂ is —NH₂ then R₃ is not —SH; when R₃ is hydrogen, R₂ is —NR₄R₅, R₄ is hydrogen then R₅ is not hydrogen, alkanyl, (C₂-C₅) alkenyl, substituted alkyl, heteroalkanyl, phenyl, para-aminophenyl, benzyl, homobenzyl, para-azidohomobenzyl,

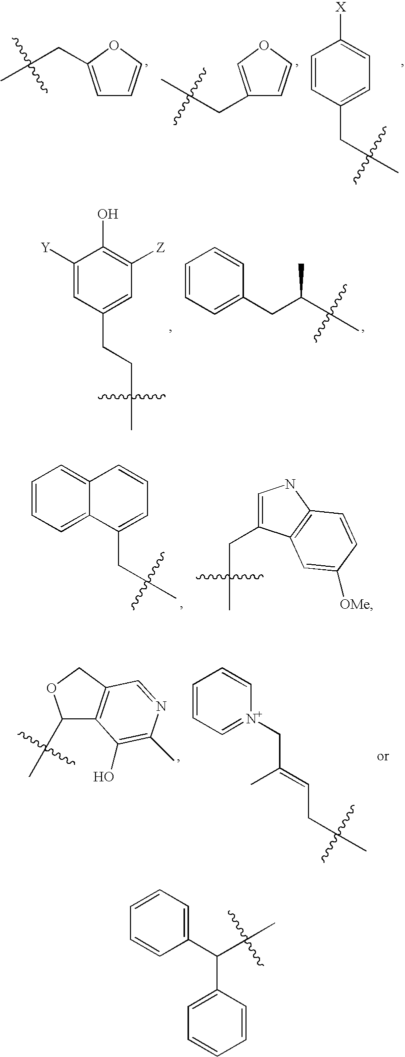

and X is —NH₂, —NO₂, —NHC(O)CH₃ or —NHC(O)CH₂Br and Y and Z are independently hydrogen or iodine;

when R₃ is hydrogen, R₂ is —NR₄R₅ and R₄ is methyl, n-butyl,

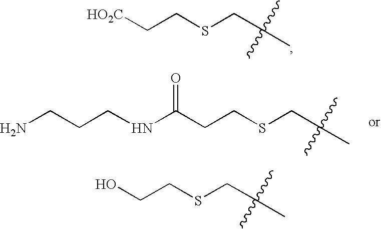

then R₅ is not methyl, n-butyl, α-napthyl, substituted alkyl,

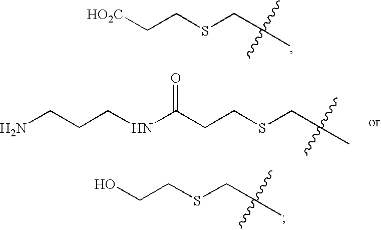

when R₃ is hydrogen and R₂ is —SR₆, then R₆ is not methyl, butyl, para-nitrobenzyl, para-aminobenzyl,

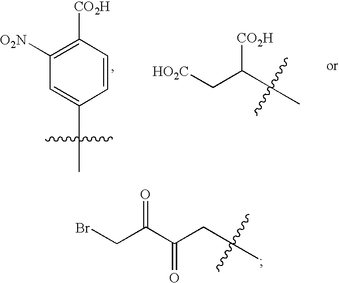

when R₂ is hydroxyl then R₃ is not

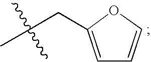

when R₃ is hydroxyl, R₂ is —NR₄R₅ and R₄ is hydrogen then R₅ is not hydrogen, methyl, butyl, C₁-C₃ substituted alkyl, —(CH₂)₄Ph, —(CH₂)₃SMe,

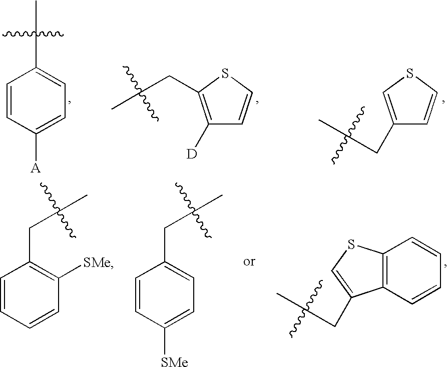

A is methyl, n-butyl, fluorine or bromine and D is hydrogen, methyl, ethyl or nitro; when R₃ is hydroxyl, R₂ is —NR₄R₅ and R₄ is methyl then R₅ is not methyl; when R₃ is hydroxyl, R₂ is —NR₄C(O)R₅ and R₄ is hydrogen then R₅ is not phenyl,

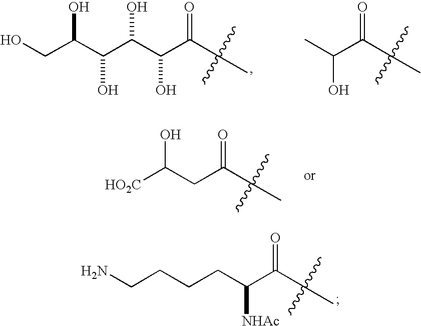

and when R₃ is —NH₂ then R₂ is not dimethylamino, methylamino, ethylamino, butylamino, acetamido or para-n-butylaniline.

In still other embodiments, when R₂ is hydrogen then R₃ is not —NH₂.

In still other embodiments, when R₂ is —NH₂ then R₃ is not —NH₂.

In some embodiments, R₄ and R₅ are independently hydrogen, alkyl, substituted alkyl, aryl, substituted aryl, arylalkyl, substituted arylalkyl, cycloalkyl, heteroalkyl, heteroarylalkyl or substituted heteroarylalkyl.

In other embodiments, R₂ is hydrogen, —NH₂, hydroxyl or —NHC(O)R₅ and R₅ is alkyl, substituted alkyl, aryl, substituted aryl, heteroaryl or substituted heteroaryl.

In still other embodiments, R₃ is hydroxyl, —NR₆R₇, —NHC(O)R₇ or —SR₆, R₆ is heteroarylalkyl, aryl, substituted aryl, arylalkyl, substituted arylalkyl, alkyl, cycloalkyl, heteroalkyl or substituted cycloheteroalkyl and R₇ is alkyl, alkyl, aryl or substituted aryl.

In still other embodiments, R₂ is hydrogen, —NH₂, hydroxyl or —NHC(O)R₅, R₅ is alkyl, substituted alkyl, aryl, substituted aryl, heteroaryl, substituted heteroaryl and R₃ is hydroxyl, —NR₆R₇, —NHC(O)R₇ or —SR₆, R₆ is heteroarylalkyl, aryl, substituted aryl, arylalkyl, substituted arylalkyl, alkyl, cycloalkyl, heteroalkyl or substituted cycloheteroalkyl and R₇ is alkyl, alkyl, aryl or substituted aryl.

In some embodiments, R₂ is —NH₂ and R₃ is hydrogen or —NH₂.

In other embodiments, R₂ is —NH₂ and R₃ is —NHR₇ and R₇ is heteroarylalkyl,

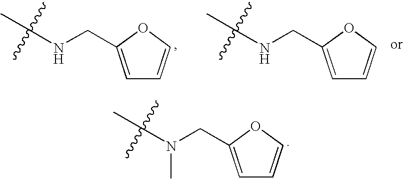

In some embodiments, R₃ is hydroxyl and R₂ is —NHC(O)R₅ and R₅ is heteroaryl or substituted heteroaryl. In other embodiments, R₅ is 2-furanyl or 2-thienyl.

In some embodiments, R₂ is —NH₂, R₃ is —NR₄R₅, R₄ is hydrogen, R₅ is

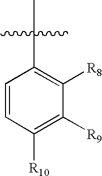

and R₈, R₉ and R₁₀ are independently hydrogen, alkoxy, alkyl or halo. In other embodiments, R₈, R₉ and R₁₀ are independently hydrogen, methoxy, methyl or fluorine. In still other embodiments, R₈ is hydrogen, methoxy, methyl or fluoro and R₉ and R₁₀ are hydrogen. In still other embodiments, R₉ is methoxy, methyl or fluoro and R₈ and R₁₀ are hydrogen. In still other embodiments, R₁₀ is methoxy, methyl or fluoro and R₈ and R₉ are hydrogen.

In some embodiments, R₂ is —NH₂ and R₃ is —NR₄R₅, R₄ is hydrogen or methyl, R₅ is

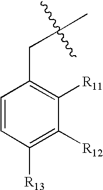

and R₁₁, R₁₂ and R₁₃ are independently hydrogen, alkoxy, alkyl or halo. In other embodiments, R₁₁, R₁₂ and R₁₃ are independently hydrogen, methoxy, methyl or fluorine. In still other embodiments, R₄ is hydrogen or methyl and R₁₁, R₁₂ and R₁₃ are hydrogen. In still other embodiments, R₄ is hydrogen and R₁₁ is methoxy, methyl or fluoro and R₁₂ and R₁₃ are hydrogen. In still other embodiments, R₄ is hydrogen and R₁₂ is methoxy, methyl or fluoro and R₁₁ and R₁₃ are hydrogen. In still other embodiments, R₄ is hydrogen and R₁₃ is methoxy, methyl or fluoro and R₁₁ and R₁₂ are hydrogen.

In some embodiments, R₂ is —NH₂ and R₃ is —NR₄R₅, R₄ and R₅ are independently hydrogen, alkyl or cycloalkyl or alternatively, R₄ and R₅ together with the atoms to which they are attached form a cycloheteroalkyl ring. In other embodiments, R₄ is hydrogen and R₅ is alkyl or cycloalkyl. In still other embodiments, R₄ is hydrogen and R₅ is isopropyl, n-butyl, n-pentyl, cyclopropyl or cyclopentyl. In still other embodiments, R₄ and R₅ together with the atoms to which they are attached form a piperidinyl or pyrrolidinyl ring.

In some embodiments, R₃ is —OH, R₂ is —NHC(O)R₅ and R₅ is alkyl, substituted alkyl, aryl, substituted aryl or cycloalkyl. In other embodiments, R₅ is

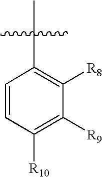

and R₈, R₉ and R₁₀ are independently hydrogen, alkoxy, alkyl, substituted alkyl or halo. In still other embodiments, R₈, R₉ and R₁₀ are independently hydrogen, fluoro, methoxy, methyl or trifluoromethyl. In still other embodiments, R₈ is methoxy or fluoro and R₉ and R₁₀ are hydrogen. In still other embodiments, R₉ is methoxy or methyl and R₈ and R₁₀ are hydrogen. In still other embodiments, R₁₀ is methoxy or trifluoromethyl and R₈ and R₉ are hydrogen. In still other embodiments, R₅ isopropyl, n-butyl, cyclohexyl or —CH₂OPh.

In some embodiments, R₂ is hydrogen and R₃ is —NR₄R₅. In other embodiments, R₄ is hydrogen, alkyl or arylalkyl and R₅ is aryl, substituted aryl, arylalkyl, substituted arylalkyl, heteroarylalkyl, heteroalkyl, cycloalkyl or substituted cycloheteroalkyl.

In some embodiments, —NR₄R₅ is R₄ is

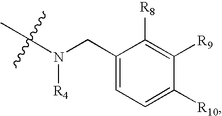

hydrogen, alkyl or arylalkyl and R₈, R₉ and R₁₀ are independently hydrogen, alkyl, alkoxy or halo. In other embodiments, R₄ is hydrogen, methyl or benzyl and R₈, R₉ and R₁₀ are hydrogen. In still other embodiments, 4 is hydrogen, R₈ is methyl and R₉ and R₁₀ are hydrogen. In still other embodiments, R₄ is hydrogen, R₉ is methyl, methoxy or fluorine and R₈ and R₁₀ are hydrogen. In still other embodiments, R₄ is hydrogen, R₁₀ is methoxy or fluorine and R₈ and R₉ are hydrogen.

In some embodiments, R₄ is hydrogen or alkyl and R₅ is alkyl, heteroalkyl, cycloalkyl, substituted cycloheteroalkyl, arylalkyl or heteroarylalkyl or alternatively, R₄ and R₅ together with the atoms to which they are attached form a cycloheteroalkyl ring. In other embodiments, R₄ and R₅ are n-propyl.

In some embodiments, R₄ is methyl and R₅ is

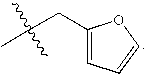

In other embodiments, R₄ is hydrogen and R₅ is methyl, ethyl, n-butyl or n-octyl. In still other embodiments, R₄ is hydrogen and R₅ is

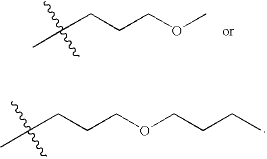

In some embodiments, R₄ is hydrogen and R₅ is

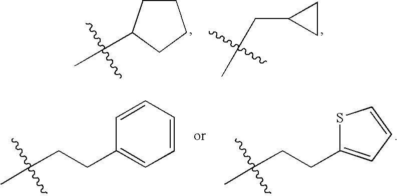

In some embodiments, R₄ and R₅ together with the atoms to which they are attached form a cycloheteroalkyl ring. In other embodiments, R₄ and R₅ together with the atoms to which they are attached form:

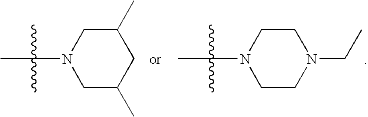

In some embodiments, R₄ is hydrogen, R₅ is

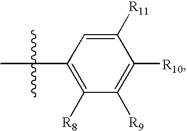

R₈, R₉, R₁₀ and R₁₁ are independently alkyl, —CH₃, alkoxy, —OCH₃, —OC₂H₅, halo, —F, —Cl or —Br, —NHCOR₁₂, R₁₂ is alkyl or substituted alkyl or —NHCOCH₃. In other embodiments, R₈ is methyl or fluoro and R₉, R₁₀ and R₁₁ are hydrogen. In still other embodiments, R₉ is methyl, methoxy, fluoro, bromo or —NHCOCH₃ and R₈, R₁₀ and R₁₁ are hydrogen. In still other embodiments, R₁₀ is methyl, n-butyl, methoxy, ethoxy, fluoro or chloro and R₈, R₉ and R₁₁ are hydrogen. In still other embodiments, R₉ and R₁₀ are methoxy, fluoro or chloro and R₈ and R₁₁ are hydrogen. In still other embodiments, R₉ is chloro, R₁₀ is methyl and R₈ and R₁₁ are hydrogen. In still other embodiments, R₉ and R₁₁ are chloro and R₈ and R₁₀ are hydrogen.

In some embodiments, R₂ is hydrogen and R₃ is —NHCOR₇. In other embodiments, R₇ is alkyl, aryl, substituted aryl or heteroaryl. In still other embodiments, R₇ is methyl, n-propyl or isopropyl. In still other embodiments, R₇ is

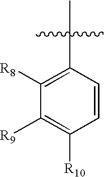

and R₈, R₉ and R₁₀ are independently hydrogen, alkoxy, alkyl or halo. In still other embodiments, R₈ is methyl, methoxy or fluoro and R₉ and R₁₀ are hydrogen. In still other embodiments, R₉ is methyl, methoxy or fluoro and R₈ and R₁₀ are hydrogen. In still other embodiments, R₁₀ is methoxy and R₉ and R₁₀ are hydrogen. In still other embodiments, R₇ is 2-furanyl.

In some embodiments, R₂ is hydrogen and R₃ is —SR₆. In other embodiments, R₆ is alkyl, heteroalkyl, arylalkyl or substituted arylalkyl. In still other embodiments, R₆ is

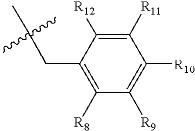

and R₈, R₉, R₁₀, R₁₁ and R₁₂ are independently alkyl, alkoxy, halo or cyano. In still other embodiments, R₈, R₉, R₁₀, R₁₁ and R₁₂ are independently methyl, methoxy, fluoro, chloro, bromo or cyano. In still other embodiments, R₈ is hydrogen, methyl, methoxy, fluoro, chloro, bromo or cyano and R₉, R₁₀, R₁₁ and R₁₂ are hydrogen. In still other embodiments, R₉ is methyl, methoxy, fluoro or cyano and R₈, R₁₀, R₁₁ and R₁₂ are hydrogen. In still other embodiments, R₁₀ is methoxy, fluoro or chloro and R₈, R₉, R₁₁ and R₁₂ are hydrogen. In still other embodiments, R₉ and R₁₀ are methyl and R₈, R₁₁ and R₁₂ are hydrogen. In still other embodiments, R₈ and R₁₁ are methyl and R₉, R₁₀ and R₁₂ are hydrogen. In still other embodiments, R₈ and R₁₀ are chloro and R₉, R₁₁ and R₁₂ are hydrogen. In still other embodiments, R₈ is chloro, R₁₂ is fluoro and R₉, R₁₀ and R₁₁ are hydrogen. In still other embodiments, R₆ is hydrogen, methyl, isopropyl, isobutyl, or

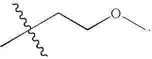

In still other embodiments, R₆ is

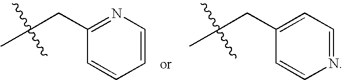

In some embodiments, R₂ is —NHCOR₅, R₃ is —OH and R₅ is aryl, substituted aryl, heteroaryl or substituted heteroaryl. In other embodiments, R₅ is

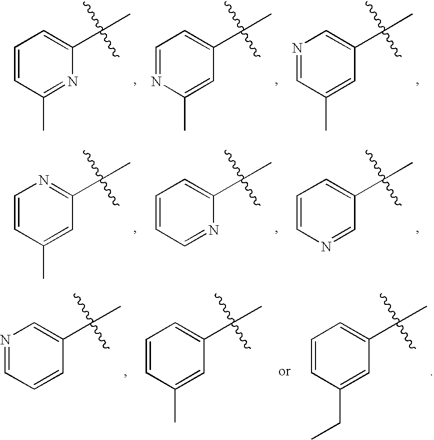

In some aspects, a compound of structural Formula (II) is provided:

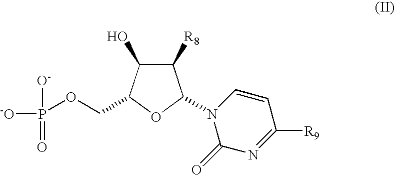

or a salt, hydrate or solvate thereof, wherein:

R₈ is hydrogen or hydroxyl;

R₉ is —NR₁₀C(O)R₁₁;

R₁₀ is hydrogen, substituted alkyl, aryl, substituted aryl, arylalkyl, substituted arylalkyl, heteroalkyl, substituted heteroalkyl, heteroaryl, substituted heteroaryl, heteroarylalkyl or substituted heteroarylalkyl; and

R₁₁ is hydrogen, (C₁-C₁₀)alkyl, substituted alkyl, aryl, substituted aryl, arylalkyl, substituted arylalkyl, heteroalkyl, substituted heteroalkyl, heteroaryl, substituted heteroaryl, heteroarylalkyl or substituted heteroarylalkyl. In some embodiments, R₉ is not

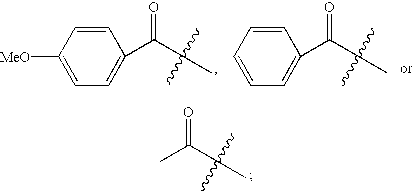

when R₈ is hydrogen then R₉ is not

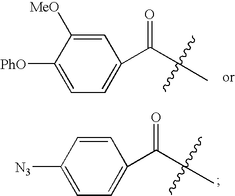

and when R₈ is hydroxyl then R₉ is not

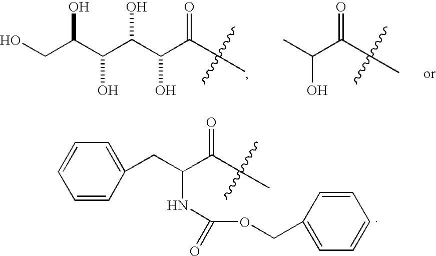

In some embodiments, R₁₀ is hydrogen and R₁₁ is heteroaryl, alkyl, substituted alkyl, aryl or substituted aryl. In other embodiments, R₈ is hydrogen, R₉ is —NR₁₀C(O)R₁₁, R₁₀ is hydrogen and R₁₁ is alkyl, substituted alkyl, aryl or substituted aryl. In still other embodiments, R₁₁ is isopropyl, t-butyl, —CH₂OPh or 3-methylphenyl. In still other embodiments, R₈ is hydrogen, R₁₀ is hydrogen and R₁₁ is 2-thienyl. In still other embodiments, R₈ is hydroxyl, R₉ is —NR₁₀C(O)R₁₁, R₁₀ is hydrogen and R₁₁ is aryl or substituted aryl. In still other embodiments, R₁₁ is phenyl, 3-methylphenyl or 4-methoxyphenyl.

In still another aspect, a compound of structural Formula (III) is provided:

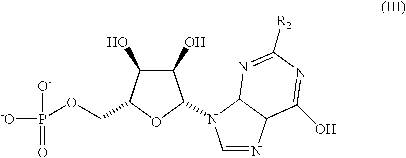

or a salt, hydrate or solvate thereof, wherein:

R₂ is hydrogen, —NR₄R₅ or —NR₄C(O)R₅; and

R₄ and R₅ are independently hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl or substituted heteroarylalkyl. In some embodiments, R₂ is hydrogen or —NH₂.

In still another aspect, a compound of structural Formula (IV) is provided:

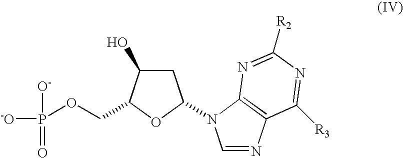

or a salt, hydrate or solvate thereof, wherein:

R₂ is hydrogen, —NR₄R₅ or —NR₄C(O)R₅;

R₄ and R₅ are independently hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl or substituted heteroarylalkyl; and

R₃ is hydroxyl, —NR₆R₇ or —NR₆C(O)R₇. In some embodiments, R₂ is hydrogen or —NR₄R₅ and R₃ is hydroxyl or —NR₆R₇. In other embodiments, R₂ is hydrogen or —NH₂ and R₃ is hydroxyl or —NH₂.

The compounds of structural formulae (I), (II), (III) and (IV) may contain one or more chiral centers and/or double bonds and therefore, may exist as stereoisomers, such as double-bond isomers (i.e., geometric isomers), enantiomers or diastereomers. In some embodiments, the chemical structures depicted herein encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan.

The compounds exemplified herein may be obtained via the synthetic methods illustrated in Schemes 1-6. Those of skill in the art will appreciate that many methods and procedures are available to synthesize nucleotides. (See e.g., Green et al., “Protective Groups in Organic Chemistry”, (Wiley, 2^{nd} ed. 1991); Harrison et al., “Compendium of Synthetic Organic Methods”, Vols. 1-8 (John Wiley and Sons, 1971-1996); “Beilstein Handbook of Organic Chemistry,” Beilstein Institute of Organic Chemistry, Frankfurt, Germany; Feiser et al., “Reagents for Organic Synthesis,” Volumes 1-17, Wiley Interscience; Trost et al., “Comprehensive Organic Synthesis,” Pergamon Press, 1991; “Theilheimer's Synthetic Methods of Organic Chemistry,” Volumes 1-45, Karger, 1991; March, “Advanced Organic Chemistry,” Wiley Interscience, 1991; Larock “Comprehensive Organic Transformations,” VCH Publishers, 1989; Paquette, “Encyclopedia of Reagents for Organic Synthesis,” John Wiley & Sons, 1995, Bodanzsky, “Principles of Peptide Synthesis,” Springer Verlag, 1984; Bodanzsky, “Practice of Peptide Synthesis,” Springer Verlag, 1984).

Starting materials useful for preparing compounds described herein and intermediates thereof are either commercially available or can be prepared by well-known synthetic methods. Other methods for synthesis of nucleotides are either described in the art or will be readily apparent to the skilled artisan in view of the references provided and may be used to synthesize these compounds. Accordingly, the methods presented in the Schemes herein are illustrative rather than comprehensive.

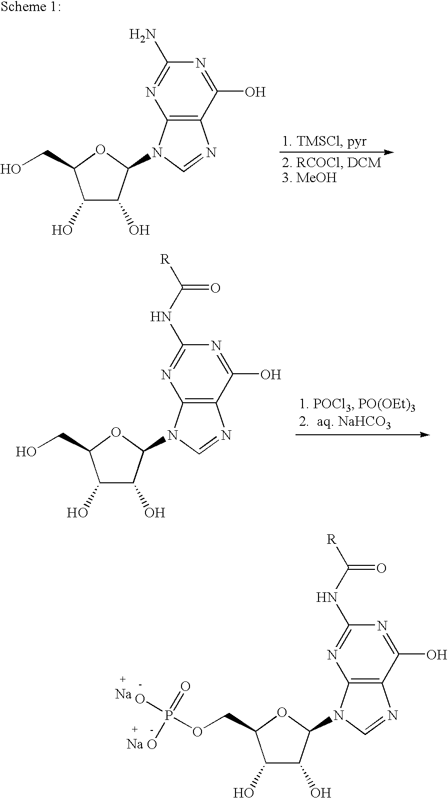

As illustrated in Scheme 1 (Fan et al., Org. Lett. 2004, 2555-2557), treatment of guanosine with trimethylsilyl chloride protects the ribose hydroxyl groups. The free amine is then acylated with a suitable acyl chloride and hydrolysis of the silyl groups provides the acylated guanosine derivative which is then converted to the primary monophosphate with phosphorus oxychloride and triethylphosphite.

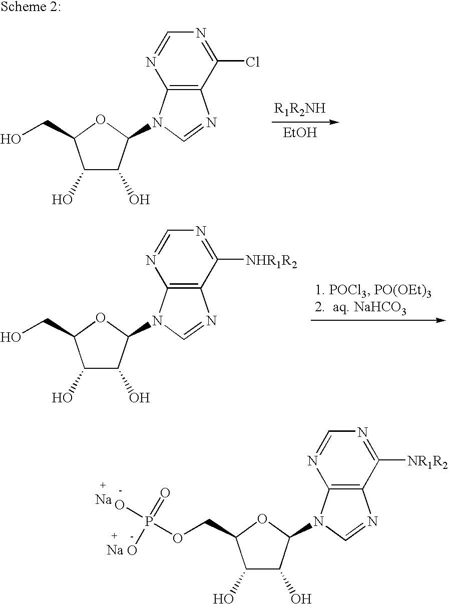

As illustrated in Scheme 2, (Trivedi et al., J. Med. Chem. 1989, 32, 1667-1673) amino purine derivatives can be readily synthesized from the chloropurine. Substitution of chlorine for amine is readily accomplished by mixing free amine with the chloropurine in ethanol. The amino purine is converted to the primary monophosphate with phosphorus oxychloride and triethylphosphite.

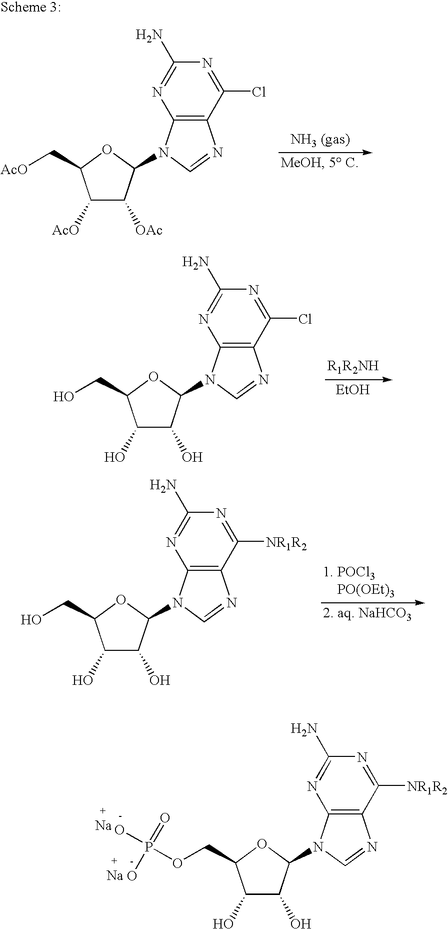

As illustrated in Scheme 3, (Trivedi et al., J. Med. Chem. 1989, 32, 1667-1673) diaminopurine derivatives can be prepared from the fully protected amino chloropurine. Ammonia in methanol hydrolyzes the acetate group of the starting material to provide the deprotected amino chloropurine which is then reacted with free amine in ethanol to yield the diaminopurine. The diaminopurine is then converted to the primary monophosphate with phosphorus oxychloride and triethylphosphite.

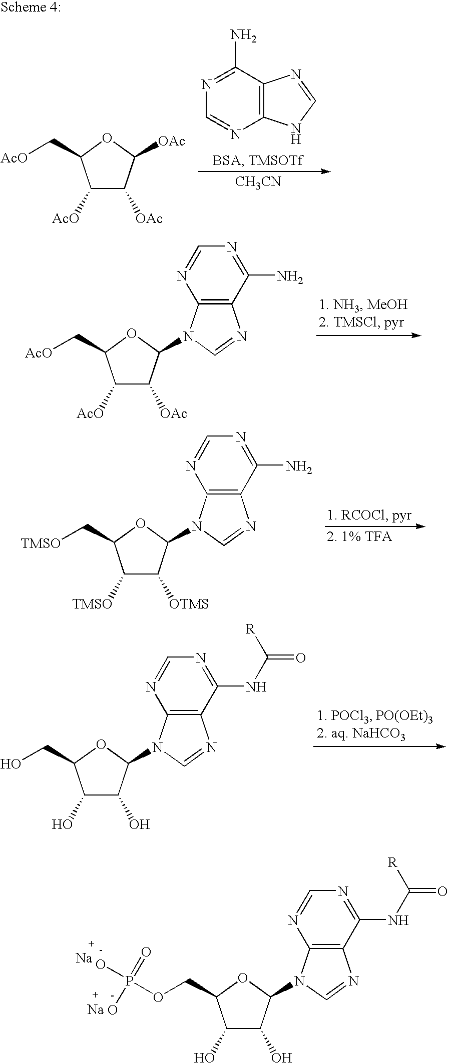

As illustrated in Scheme 4, attachment of adenine to the anomeric carbon of peracetylated ribose provides an adenosine derivative. Hydrolysis of the acetate groups followed by reaction with trimethylsilyl chloride yields the persilylated adenosine which is then reacted with a suitable acyl chloride. Hydrolysis of the trimethylsilyl group and reaction with phosphorus oxychloride and triethylphosphite provides the acylated adenosine monophosphates.

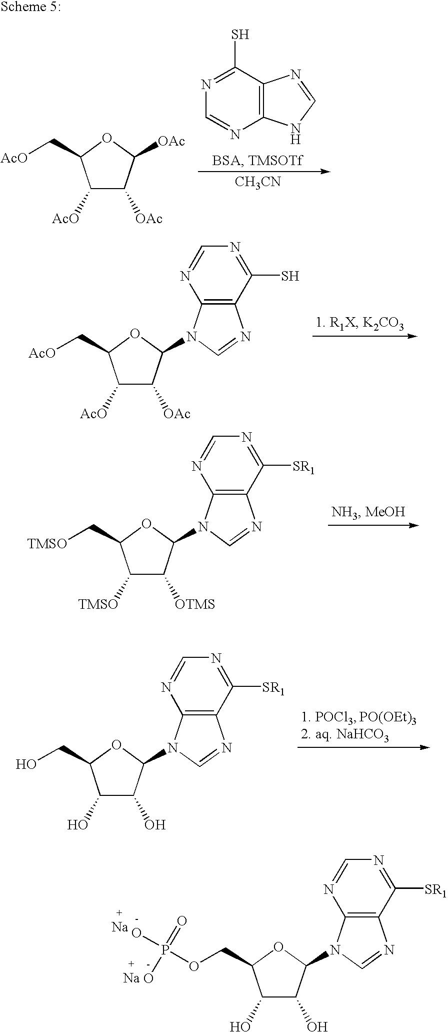

As illustrated in Scheme 5, thio-adenine is attached to the anomeric carbon of peracetylated ribose under standard conditions. Reaction of the thiol with an appropriate alkyl halide under conventional conditions provides the alkylated thiol which is then deprotected and converted to the monophosphate as previously described.

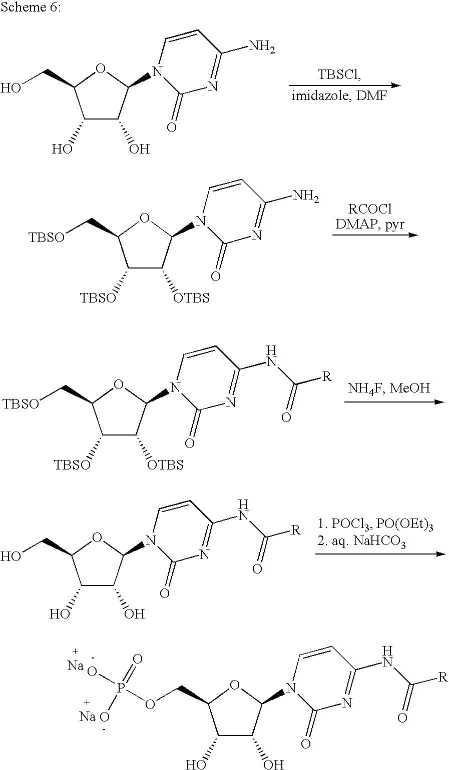

As illustrated in Scheme 6, the hydroxyl groups of cytosine are fully protected and the amino group is acylated with an appropriate acyl chloride. Deprotection and reaction with phosphorus oxychloride and triethylphosphite provides the acylated cytosine monophosphate derivatives.

In general, chemosensory receptor modifiers or chemosensory receptor ligand modifiers of the present invention are provided in a composition, e.g., pharmaceutical, medicinal or comestible composition, or alternatively, in a formulation, e.g., a pharmaceutical or medicinal formulation or a food or beverage product or formulation.

In one embodiment, the chemosensory receptor modifiers or chemosensory receptor ligand modifiers provided by the present invention can be used at very low concentrations on the order of a few parts per million, in combination with one or more umami flavor entities, natural or artificial, so as to reduce the concentration of the known umami flavor entity required to prepare a comestible composition having the desired degree of savory taste.

In yet another embodiment, the chemosensory receptor modifier and chemosensory receptor ligand modifier can be formulated, individually or in combination, in flavor preparations to be added to food and beverage formulations or products.

Typically at least a chemosensory receptor modulating amount, a chemosensory receptor ligand modulating amount, a umami flavor modulating amount, a umami flavoring agent amount, or a umami flavor enhancing amount of one or more of the chemosensory receptor modifiers or chemosensory receptor ligand modifiers of the present invention will be added to the comestible or medicinal product, optionally in the presence of one or more other umami flavor entities so that the umami flavor modified comestible or medicinal product has an increased umami taste as compared to the comestible or medicinal product prepared without the modifiers of the present invention, as judged by human beings or animals in general, or in the case of formulations testing, as judged by a majority of a panel of at least eight human taste testers, via procedures commonly known in the field.

The concentration of umami flavoring agent needed to modulate or improve the flavor of the comestible or medicinal product or composition will of course depend on many variables, including the specific type of comestible composition and its various other ingredients, especially the presence of other known umami flavoring agents and the concentrations thereof, the natural genetic variability and individual preferences and health conditions of various human beings tasting the compositions, and the subjective effect of the particular compound on the taste of such chemosensory compounds.

One application of the chemosensory receptor modifiers and/or chemosensory receptor ligand modifiers is for modulating (inducing, enhancing or inhibiting) the umami taste or other taste properties of other natural or synthetic umami tastants, and comestible compositions made therefrom. A broad but also low range of concentrations of the compounds or entities of the present invention would typically be required, i.e., from about 0.001 ppm to 100 ppm, or narrower alternative ranges from about 0.1 ppm to about 10 ppm, from about 0.01 ppm to about 30 ppm, from about 0.05 ppm to about 10 ppm, from about 0.01 ppm to about 5 ppm, or from about 0.02 ppm to about 2 ppm, or from about 0.01 ppm to about 1 ppm.

In one embodiment, chemosensory receptor modifiers and chemosensory receptor ligand modifiers for the present invention, e.g., flavor modifiers, flavoring agents, flavor enhancers, umami (savory) flavoring agents and/or flavor enhancers can be used in foods, beverages and any other comestible compositions wherein savory compounds are conventionally utilized. These compositions include compositions for human and animal consumption.

Those of ordinary skill in the art of preparing and selling comestible compositions, e.g., edible foods or beverages, or precursors or flavor modifiers thereof are well aware of a large variety of classes, subclasses and species of the comestible compositions, and utilize well-known and recognized terms of art to refer to those comestible compositions while endeavoring to prepare and sell various of those compositions. Such a list of terms of art is enumerated below, and it is specifically contemplated hereby that the various subgenuses and species of the compounds of the present invention could be used to modify or enhance the savory flavor of the following list comestible compositions, either singly or in all reasonable combinations or mixtures thereof.

Exemplary comestible compositions include one or more confectioneries, chocolate confectionery, tablets, countlines, bagged selflines/softlines, boxed assortments, standard boxed assortments, twist wrapped miniatures, seasonal chocolate, chocolate with toys, alfajores, other chocolate confectionery, mints, standard mints, power mints, boiled sweets, pastilles, gums, jellies and chews, toffees, caramels and nougat, medicated confectionery, lollipops, liquorice, other sugar confectionery, gum, chewing gum, sugarised gum, sugar-free gum, functional gum, bubble gum, bread, packaged/industrial bread, unpackaged/artisanal bread, pastries, cakes, packaged/industrial cakes, unpackaged/artisanal cakes, cookies, chocolate coated biscuits, sandwich biscuits, filled biscuits, savoury biscuits and crackers, bread substitutes, breakfast cereals, rte cereals, family breakfast cereals, flakes, muesli, other rte cereals, children's breakfast cereals, hot cereals, ice cream, impulse ice cream, single portion dairy ice cream, single portion water ice cream, multi-pack dairy ice cream, multi-pack water ice cream, take-home ice cream, take-home dairy ice cream, ice cream desserts, bulk ice cream, take-home water ice cream, frozen yoghurt, artisanal ice cream, dairy products, milk, fresh/pasteurised milk, full fat fresh/pasteurised milk, semi skimmed fresh/pasteurised milk, long-life/uht milk, full fat long life/uht milk, semi skimmed long life/uht milk, fat-free long life/uht milk, goat milk, condensed/evaporated milk, plain condensed/evaporated milk, flavoured, functional and other condensed milk, flavoured milk drinks, dairy only flavoured milk drinks, flavoured milk drinks with fruit juice, soy milk, sour milk drinks, fermented dairy drinks, coffee whiteners, powder milk, flavoured powder milk drinks, cream, cheese, processed cheese, spreadable processed cheese, unspreadable processed cheese, unprocessed cheese, spreadable unprocessed cheese, hard cheese, packaged hard cheese, unpackaged hard cheese, yoghurt, plain/natural yoghurt, flavoured yoghurt, fruited yoghurt, probiotic yoghurt, drinking yoghurt, regular drinking yoghurt, probiotic drinking yoghurt, chilled and shelf-stable desserts, dairy-based desserts, soy-based desserts, chilled snacks, fromage frais and quark, plain fromage frais and quark, flavoured fromage frais and quark, savoury fromage frais and quark, sweet and savoury snacks, fruit snacks, chips/crisps, extruded snacks, tortilla/corn chips, popcorn, pretzels, nuts, other sweet and savoury snacks, snack bars, granola bars, breakfast bars, energy bars, fruit bars, other snack bars, meal replacement products, slimming products, convalescence drinks, ready meals, canned ready meals, frozen ready meals, dried ready meals, chilled ready meals, dinner mixes, frozen pizza, chilled pizza, soup, canned soup, dehydrated soup, instant soup, chilled soup, uht soup, frozen soup, pasta, canned pasta, dried pasta, chilled/fresh pasta, noodles, plain noodles, instant noodles, cups/bowl instant noodles, pouch instant noodles, chilled noodles, snack noodles, canned food, canned meat and meat products, canned fish/seafood, canned vegetables, canned tomatoes, canned beans, canned fruit, canned ready meals, canned soup, canned pasta, other canned foods, frozen food, frozen processed red meat, frozen processed poultry, frozen processed fish/seafood, frozen processed vegetables, frozen meat substitutes, frozen potatoes, oven baked potato chips, other oven baked potato products, non-oven frozen potatoes, frozen bakery products, frozen desserts, frozen ready meals, frozen pizza, frozen soup, frozen noodles, other frozen food, dried food, dessert mixes, dried ready meals, dehydrated soup, instant soup, dried pasta, plain noodles, instant noodles, cups/bowl instant noodles, pouch instant noodles, chilled food, chilled processed meats, chilled fish/seafood products, chilled processed fish, chilled coated fish, chilled smoked fish, chilled lunch kit, chilled ready meals, chilled pizza, chilled soup, chilled/fresh pasta, chilled noodles, oils and fats, olive oil, vegetable and seed oil, cooking fats, butter, margarine, spreadable oils and fats, functional spreadable oils and fats, sauces, dressings and condiments, tomato pastes and pures, bouillon/stock cubes, stock cubes, gravy granules, liquid stocks and fonds, herbs and spices, fermented sauces, soy based sauces, pasta sauces, wet sauces, dry sauces/powder mixes, ketchup, mayonnaise, regular mayonnaise, mustard, salad dressings, regular salad dressings, low fat salad dressings, vinaigrettes, dips, pickled products, other sauces, dressings and condiments, baby food, milk formula, standard milk formula, follow-on milk formula, toddler milk formula, hypoallergenic milk formula, prepared baby food, dried baby food, other baby food, spreads, jams and preserves, honey, chocolate spreads, nut-based spreads, and yeast-based spreads.

In another example, the compounds of the present invention can be used to modify or enhance the savory flavor of one or more of the following sub-genuses of comestible compositions: confectioneries, bakery products, ice creams, dairy products, sweet and savory snacks, snack bars, meal replacement products, ready meals, soups, pastas, noodles, canned foods, frozen foods, dried foods, chilled foods, oils and fats, baby foods, or spreads, or a mixture thereof.

In yet another example, the compounds of the present invention can be incorporated in foods and beverages, e.g., foods and beverages in Wet Soup Category, the Dehydrated and Culinary Food Category, the Beverage Category, the Frozen Food Category, the Snack Food Category, and seasonings or seasoning blends.

In general, “Wet Soup Category” means wet/liquid soups regardless of concentration or container, including frozen Soups. For the purpose of this definition soup(s) means a food prepared from meat, poultry, fish, vegetables, grains, fruit and other ingredients, cooked in a liquid which may include visible pieces of some or all of these ingredients. It may be clear (as a broth) or thick (as a chowder), smooth, pureed or chunky, ready-to-serve, semi-condensed or condensed and may be served hot or cold, as a first course or as the main course of a meal or as a between meal snack (sipped like a beverage). Soup may be used as an ingredient for preparing other meal components and may range from broths (consomm) to sauces (cream or cheese-based soups).

“Dehydrated and Culinary Food Category” usually means: (i) cooking aid products such as: powders, granules, pastes, concentrated liquid products, including concentrated bouillon, bouillon and bouillon like products in pressed cubes, tablets or powder or granulated form, which are sold separately as a finished product or as an ingredient within a product, sauces and recipe mixes (regardless of technology); (ii) meal solutions products such as: dehydrated and freeze dried soups, including dehydrated soup mixes, dehydrated instant soups, dehydrated ready-to-cook soups, dehydrated or ambient preparations of ready-made dishes, meals and single serve entrees including pasta, potato and rice dishes; and (iii) meal embellishment products such as: condiments, marinades, salad dressings, salad toppings, dips, breading, batter mixes, shelf stable spreads, barbecue sauces, liquid recipe mixes, concentrates, sauces or sauce mixes, including recipe mixes for salad, sold as a finished product or as an ingredient within a product, whether dehydrated, liquid or frozen.

“Beverage Category” usually means beverages, beverage mixes and concentrates, including but not limited to, alcoholic and non-alcoholic ready to drink and dry powdered beverages.

Other examples of foods and beverages wherein compounds of the present invention may be incorporated included by way of example carbonated and non-carbonated beverages, e.g., sodas, fruit or vegetable juices, alcoholic and non-alcoholic beverages, confectionary products, e.g., cakes, cookies, pies, candies, chewing gums, gelatins, ice creams, sorbets, puddings, jams, jellies, salad dressings, and other condiments, cereal, and other breakfast foods, canned fruits and fruit sauces and the like.

In still another example, the compounds of the present invention can be combined with or applied to the comestible or medicinal products or precursor thereof in any of innumerable ways known or later discovered. For example, the compounds of the present invention could be dissolved in or dispersed in or one of many comestibly acceptable liquids, solids, or other carriers, such as water at neutral, acidic, or basic pH, fruit or vegetable juices, vinegar, marinades, beer, wine, natural water/fat emulsions such as milk or condensed milk, edible oils and shortenings, fatty acids, certain low molecular weight oligomers of propylene glycol, glyceryl esters of fatty acids, and dispersions or emulsions of such hydrophobic substances in aqueous media, salts such as sodium chloride, vegetable flours, solvents such as ethanol, solid edible diluents such as vegetable powders or flours, and the like, and then combined with precursors of the comestible or medicinal products, or applied directly to the comestible or medicinal products.

In yet another embodiment, the chemosensory receptor modifier and chemosensory receptor ligand modifier of the present invention can be provided in medicinal or pharmaceutical compositions containing a therapeutically effective amount of one or more compounds of the present invention, preferably in purified form, together with a suitable amount of a medicinally or pharmaceutically acceptable vehicle, so as to provide the form for proper administration to a patient or person in need of such administration.

When administered to a patient or a person in need of administration, the compounds of the present invention and pharmaceutically acceptable vehicles are preferably sterile. Water is a preferred vehicle when a compound of the present invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present pharmaceutical compositions, if desired, can also contain minor amounts of wetting or emulsifying agents, or pH buffering agents. In addition, auxiliary, stabilizing, thickening, lubricating and coloring agents may be used.

Pharmaceutical compositions comprising a compound of the present invention may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries, which facilitate processing of compounds of the present invention into preparations which can be used pharmaceutically. Proper formulation is dependent upon the route of administration chosen.

The present pharmaceutical compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In some embodiments, the pharmaceutically acceptable vehicle is a capsule (see e.g., Grosswald et al., U.S. Pat. No. 5,698,155). Other examples of suitable pharmaceutical vehicles have been described in the art (see Remington: The Science and Practice of Pharmacy, Philadelphia College of Pharmacy and Science, 20^{th} Edition, 2000).

For topical administration a compound of the present invention may be formulated as solutions, gels, ointments, creams, suspensions, etc. as is well-known in the art.

Systemic formulations include those designed for administration by injection, e.g., subcutaneous, intravenous, intramuscular, intrathecal or intraperitoneal injection, as well as those designed for transdermal, transmucosal, oral or pulmonary administration. Systemic formulations may be made in combination with a further active agent that improves mucociliary clearance of airway mucus or reduces mucous viscosity. These active agents include, but are not limited to, sodium channel blockers, antibiotics, N-acetyl cysteine, homocysteine and phospholipids.

In some embodiments, the compounds of the present invention are formulated in accordance with routine procedures as a pharmaceutical composition adapted for intravenous administration to human beings. Typically, compounds of the present invention for intravenous administration are solutions in sterile isotonic aqueous buffer. For injection, a compound of the present invention may be formulated in aqueous solutions, preferably in physiologically compatible buffers such as Hanks' solution, Ringer's solution, or physiological saline buffer. The solution may contain formulatory agents such as suspending, stabilizing and/or dispersing agents. When necessary, the pharmaceutical compositions may also include a solubilizing agent.

Pharmaceutical compositions for intravenous administration may optionally include a local anesthetic such as lignocaine to ease pain at the site of the injection. Generally, the ingredients are supplied either separately or mixed together in unit dosage form, for example, as a lyophilized powder or water free concentrate in a hermetically sealed container such as an ampoule or sachette indicating the quantity of active agent. When the compound of the present invention is administered by infusion, it can be dispensed, for example, with an infusion bottle containing sterile pharmaceutical grade water or saline. When the compound of the present invention is administered by injection, an ampoule of sterile water for injection or saline can be provided so that the ingredients may be mixed prior to administration.

For transmucosal administration, penetrants appropriate to the barrier to be permeated are used in the formulation. Such penetrants are generally known in the art.

Pharmaceutical compositions for oral delivery may be in the form of tablets, lozenges, aqueous or oily suspensions, granules, powders, emulsions, capsules, syrups, or elixirs, for example. Orally administered pharmaceutical compositions may contain one or more optionally agents, for example, sweetener agents such as fructose, aspartame or saccharin; flavoring agents such as peppermint, oil of wintergreen, or cherry coloring agents and preserving agents, to provide a pharmaceutically palatable preparation.

Moreover, where in tablet or pill form, the pharmaceutical compositions may be coated to delay disintegration and absorption in the gastrointestinal tract, thereby providing a sustained action over an extended period of time. Selectively permeable membranes surrounding an osmotically active driving compound are also suitable for orally administered compounds of the present invention. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade.

For oral liquid preparations such as, for example, suspensions, elixirs and solutions, suitable carriers, excipients or diluents include water, saline, alkyleneglycols (e.g., propylene glycol), polyalkylene glycols (e.g., polyethylene glycol) oils, alcohols, slightly acidic buffers between pH 4 and pH 6 (e.g., acetate, citrate, ascorbate at between about 5.0 mM to about 50.0 mM) etc. Additionally, flavoring agents, preservatives, coloring agents, bile salts, acylcamitines and the like may be added.

For buccal administration, the pharmaceutical compositions may take the form of tablets, lozenges, etc. formulated in conventional manner.

Liquid drug formulations suitable for use with nebulizers and liquid spray devices and EHD aerosol devices will typically include a compound of the present invention with a pharmaceutically acceptable vehicle. Preferably, the pharmaceutically acceptable vehicle is a liquid such as alcohol, water, polyethylene glycol or a perfluorocarbon. Optionally, another material may be added to alter the aerosol properties of the solution or suspension of compounds of the invention. Preferably, this material is liquid such as an alcohol, glycol, polyglycol or a fatty acid. Other methods of formulating liquid drug solutions or suspension suitable for use in aerosol devices are known to those of skill in the art (see, e.g., Biesalski, U.S. Pat. No. 5,112,598; Biesalski, U.S. Pat. No. 5,556,611).

A compound of the present invention may also be formulated in rectal or vaginal pharmaceutical compositions such as suppositories or retention enemas, e.g., containing conventional suppository bases such as cocoa butter or other glycerides.

In addition to the formulations described previously, a compound of the present invention may also be formulated as a depot preparation. Such long acting formulations may be administered by implantation (for example subcutaneously or intramuscularly) or by intramuscular injection. Thus, for example, a compound of the present invention may be formulated with suitable polymeric or hydrophobic materials (for example, as an emulsion in an acceptable oil) or ion exchange resins, or as sparingly soluble derivatives, for example, as a sparingly soluble salt.

When a compound of the present invention is acidic, it may be included in any of the above-described formulations as the free acid, a pharmaceutically acceptable salt, a solvate or hydrate. Pharmaceutically acceptable salts substantially retain the activity of the free acid, may be prepared by reaction with bases and tend to be more soluble in aqueous and other protic solvents than the corresponding free acid form.

A compound of the present invention, and/or pharmaceutical composition thereof, will generally be used in an amount effective to achieve the intended purpose. For use to treat or prevent diseases or disorders the compounds of the present invention and/or pharmaceutical compositions thereof, are administered or applied in a therapeutically effective amount.

The amount of a compound of the present invention that will be effective in the treatment of a particular disorder or condition disclosed herein will depend on the nature of the disorder or condition and can be determined by standard clinical techniques known in the art, as previously described. In addition, in vitro or in vivo assays may optionally be employed to help identify optimal dosage ranges. The amount of a compound of the present invention administered will, of course, be dependent on, among other factors, the subject being treated, the weight of the subject, the severity of the affliction, the manner of administration and the judgment of the prescribing physician.

For example, the dosage may be delivered in a pharmaceutical composition by a single administration, by multiple applications or controlled release. In some embodiment, the compounds of the present invention are delivered by oral sustained release administration. Dosing may be repeated intermittently, may be provided alone or in combination with other drugs and may continue as long as required for effective treatment of the disease state or disorder.

Suitable dosage ranges for oral administration depend on potency, but are generally between about 0.001 mg to about 200 mg of a compound of the present invention per kilogram body weight. Dosage ranges may be readily determined by methods known to the artisan of ordinary skill the art.

Suitable dosage ranges for intravenous (i.v.) administration are about 0.01 mg to about 100 mg per kilogram body weight. Suitable dosage ranges for intranasal administration are generally about 0.01 mg/kg body weight to about 1 mg/kg body weight. Suppositories generally contain about 0.01 milligram to about 50 milligrams of a compound of the present invention per kilogram body weight and comprise active ingredient in the range of about 0.5% to about 10% by weight. Recommended dosages for intradermal, intramuscular, intraperitoneal, subcutaneous, epidural, sublingual or intracerebral administration are in the range of about 0.001 mg to about 200 mg per kilogram of body weight. Effective doses may be extrapolated from dose-response curves derived from in vitro or animal model test systems. Such animal models and systems are well-known in the art.

Preferably, a therapeutically effective dose of a compound of the present invention described herein will provide therapeutic benefit without causing substantial toxicity. Toxicity of compounds of the present invention may be determined using standard pharmaceutical procedures and may be readily ascertained by the skilled artisan. The dose ratio between toxic and therapeutic effect is the therapeutic index. A compound of the present invention will preferably exhibit particularly high therapeutic indices in treating disease and disorders. The dosage of a compound of the present invention described herein will preferably be within a range of circulating concentrations that include an effective dose with little or no toxicity.

In certain embodiments of the present invention, the compounds of the present invention and/or pharmaceutical compositions thereof can be used in combination therapy with at least one other agent. The compound of the present invention and/or pharmaceutical composition thereof and the other agent can act additively or, more preferably, synergistically. In some embodiments, a compound of the present invention and/or pharmaceutical composition thereof is administered concurrently with the administration of another agent, which may be part of the same pharmaceutical composition as the compound of the present invention or a different pharmaceutical composition. In other embodiments, a pharmaceutical composition of the present invention is administered prior or subsequent to administration of another agent.

In still another embodiment, the chemosensory receptor modifiers and chemosensory receptor ligand modifiers of the present invention and/or pharmaceutical compositions thereof may be advantageously used in human medicine.

When used to treat and/or prevent diseases or disorders, the compounds described herein and/or pharmaceutical compositions may be administered or applied singly, or in combination with other agents. The compounds and/or pharmaceutical compositions thereof may also be administered or applied singly, in combination with other active agents.

Methods of treatment and prophylaxis by administration to a patient of a therapeutically effective amount of a compound described herein and/or pharmaceutical composition thereof are provided herein. The patient may be an animal, more preferably, a mammal and most preferably, a human.

In one example, the compounds described herein and/or pharmaceutical compositions thereof, are administered orally. The compounds of the present invention and/or pharmaceutical compositions thereof may also be administered by any other convenient route, for example, by infusion or bolus injection, by absorption through epithelial or mucocutaneous linings (e.g., oral mucosa, rectal and intestinal mucosa, etc.). Administration can be systemic or local. Various delivery systems are known, (e.g., encapsulation in liposomes, microparticles, microcapsules, capsules, etc.) that can be used to administer a compound described herein and/or pharmaceutical composition thereof. Methods of administration include, but are not limited to, intradermal, intramuscular, intraperitoneal, intravenous, subcutaneous, intranasal, epidural, oral, sublingual, intranasal, intracerebral, intravaginal, transdermal, rectal, inhalation, or topical, particularly to the ears, nose, eyes, or skin. The preferred mode of administration is left to the discretion of the practitioner and will depend in-part upon the site of the medical condition. In most instances, administration will result in the release of the compounds and/or pharmaceutical compositions thereof into the bloodstream.

In another example, it may be desirable to administer one or more compounds of the present invention and/or pharmaceutical composition thereof locally to the area in need of treatment. This may be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of the condition.

In yet another example, it may be desirable to introduce one or more compounds of the present invention and/or pharmaceutical compositions thereof into the central nervous system by any suitable route, including intraventricular, intrathecal and epidural injection. Intraventricular injection may be facilitated by an intraventricular catheter, for example, attached to a reservoir, such as an Ommaya reservoir.

A compound of the present invention and/or pharmaceutical composition thereof may also be administered directly to the lung by inhalation. For administration by inhalation, a compound of the present invention and/or pharmaceutical composition thereof may be conveniently delivered to the lung by a number of different devices. For example, a Metered Dose Inhaler (“MDI”), which utilizes canisters that contain a suitable low boiling propellant, (e.g., dichlorodifluoromethane, trichlorofluoromethane, dichlorotetrafluoroethane, carbon dioxide or any other suitable gas) may be used to deliver compounds of the present invention and/or pharmaceutical compositions thereof directly to the lung.

Alternatively, a Dry Powder Inhaler (“DPI”) device may be used to administer a compound of the invention and/or pharmaceutical composition thereof to the lung. DPI devices typically use a mechanism such as a burst of gas to create a cloud of dry powder inside a container, which may then be inhaled by the patient. DPI devices are also well known in the art. A popular variation is the multiple dose DPI (“MDDPI”) system, which allows for the delivery of more than one therapeutic dose. For example, capsules and cartridges of gelatin for use in an inhaler or insufflator may be formulated containing a powder mix of a compound of the present invention and a suitable powder base such as lactose or starch for these systems.

Another type of device that may be used to deliver a compound of the present invention and/or pharmaceutical composition thereof to the lung is a liquid spray device supplied, for example, by Aradigm Corporation, Hayward, Calif. Liquid spray systems use extremely small nozzle holes to aerosolize liquid drug formulations that may then be directly inhaled into the lung.

In yet another example, a nebulizer is used to deliver a compound of the present invention and/or pharmaceutical composition thereof to the lung. Nebulizers create aerosols from liquid drug formulations by using, for example, ultrasonic energy to form fine particles that may be readily inhaled (see e.g., Verschoyle et al., British J. Cancer, 1999, 80, Suppl. 2, 96). Examples of nebulizers include devices supplied by Sheffield Pharmaceuticals, Inc (See, Armer et al., U.S. Pat. No. 5,954,047; van der Linden et al, U.S. Pat. No. 5,950,619; van der Linden et al., U.S. Pat. No. 5,970,974), and Batelle Pulmonary Therapeutics, Columbus, Ohio.

In yet another example, an electrohydrodynamic (“EHD”) aerosol device is used to deliver a compound of the present invention and/or pharmaceutical composition thereof to the lung. EHD aerosol devices use electrical energy to aerosolize liquid drug solutions or suspensions (see e.g., Noakes et al., U.S. Pat. No. 4,765,539). The electrochemical properties of the formulation may be important parameters to optimize when delivering a compound of the present invention and/or pharmaceutical composition thereof to the lung with an EHD aerosol device and such optimization is routinely performed by one of skill in the art. EHD aerosol devices may more efficiently deliver compounds to the lung than other pulmonary delivery technologies.

In yet another example, the compounds of the present invention and/or pharmaceutical compositions thereof can be delivered in a vesicle, in particular a liposome (Langer, 1990, Science 249:1527-1533; Treat et al., in “Liposomes in the Therapy of Infectious Disease and Cancer,” Lopez-Berestein and Fidler (eds.), Liss, New York, pp. 353-365 (1989); see generally “Liposomes in the Therapy of Infectious Disease and Cancer,” Lopez-Berestein and Fidler (eds.), Liss, New York, pp. 353-365 (1989)).

In yet another example, the compounds of the present invention and/or pharmaceutical compositions thereof can be delivered via sustained release systems, preferably oral sustained release systems. In one embodiment, a pump may be used (See, Langer, supra, Sefton, 1987, CRC Crit. Ref Biomed Eng. 14:201; Saudek et al., 1989, N. Engl. J. Med. 321:574).

In yet another example, polymeric materials can be used (see “Medical Applications of Controlled Release,” Langer and Wise (eds.), CRC Pres., Boca Raton, Fla. (1974); “Controlled Drug Bioavailability,” Drug Product Design and Performance, Smolen and Ball (eds.), Wiley, New York (1984); Langer et al., 1983, J Macromol. Sci. Rev. Macromol Chem. 23:61; see also Levy et al., 1985, Science 228: 190; During et al., 1989, Ann. Neurol. 25:351; Howard et al, 1989, J. Neurosurg. 71:105).

In still other embodiments, polymeric materials are used for oral sustained release delivery. Preferred polymers include sodium carboxymethylcellulose, hydroxypropylcellulose, hydroxypropylmethylcellulose and hydroxyethylcellulose (most preferred, hydroxypropyl methylcellulose). Other preferred cellulose ethers have been described (Alderman, Int. J. Pharm. Tech. & Prod. Mfr., 1984, 5(3) 1-9). Factors affecting drug release are well known to the skilled artisan and have been described in the art (Bamba et al., Int. J. Pharm 1979, 2, 307).

In yet another example, enteric-coated preparations can be used for oral sustained release administration. Preferred coating materials include polymers with a pH-dependent solubility (i.e., pH-controlled release), polymers with a slow or pH-dependent rate of swelling, dissolution or erosion (i.e., time-controlled release), polymers that are degraded by enzymes (i.e., enzyme-controlled release) and polymers that form firm layers that are destroyed by an increase in pressure (i.e., pressure-controlled release).

In still another example, osmotic delivery systems are used for oral sustained release administration (Verma et al., Drug Dev. Ind. Pharm. 2000, 26:695-708). In yet other embodiments, OROS™ osmotic devices are used for oral sustained release delivery devices (Theeuwes et al., U.S. Pat. No. 3,845,770; Theeuwes et al., U.S. Pat. No. 3,916,899).

In still another example, a controlled-release system can be placed in proximity of the target of the compounds and/or pharmaceutical composition of the invention, thus requiring only a fraction of the systemic dose (See, e.g., Goodson, in “Medical Applications of Controlled Release,” supra, vol. 2, pp. 115-138 (1984). Other controlled-release systems discussed in Langer, 1990, Science 249:1527-1533 may also be used.

Having now generally described the invention, the same will be more readily understood by reference to the following examples, which are provided by way of illustration and are not intended as limiting. It is understood that various modifications and changes can be made to the herein disclosed exemplary embodiments without departing from the spirit and scope of the invention.

### EXAMPLES

### Experiment 1

### Modeling and Identification of Potential Chemosensory Receptor Ligand Enhancer

General Procedure

The general procedures for identifying a potential chemosensory receptor ligand enhancer is summarized as the following.

1. Constructing a model of the structure of the Venus flytrap T1R1 domain

2. Docking a chemosensory receptor ligand, e.g., a umami flavor entity into the active site of the structure of the Venus flytrap domain of T1R1, with or without T1R3 present

3. Docking a chemosensory receptor ligand enhancer, e.g., a umami taste enhancer into the active site in the presence of the chemosensory receptor ligand, e.g., the umami flavor entity

4. Selecting a chemosensory receptor ligand enhancer, e.g., umami taste enhancer candidate based on two criteria: a) it fits the active site in the model, and b) it forms productive interactions with the Venus flytrap domain of T1R1 and with the chemosensory receptor ligand, e.g., the umami taste entity. Interactions can be van der Waals, burial of hydrophobic atoms or atomic groups, hydrogen bonds, ring stacking interactions, or salt-bridging electrostatic interactions. Key residues for such interactions include the hinge residues, the near active site, the pincer residues, e.g., interacting residues described in the present invention. Candidates are not restricted to fitting completely within the active site, as it is open and chemosensory receptor ligand enhancer candidates may extend beyond the active site as long as they partially extend into it.

Model of the Structure

A model of the structure of the Venus Flytrap T1R1 domain may come from crystal structures of T1R1 or of T1R1 complexed with T1R3. The domains may be in open or in closed form, and may or may not be APO or contain a ligand. Alternatively a model of the structure of the Venus Flytrap T1R1 domain may be built using standard homology modeling methods using crystal structures of available Venus flytrap domains such as the mGluR receptor Venus flytrap domains as templates to construct the model.

An example of a procedure for building such a model is to use the commercial software Homology or Modeller from the Accelrys Corporation that is well documented in the literature and available commercially. Alternative conformations of the model may further be explored using additional molecular mechanical techniques that may include but are not limited to normal mode analysis to explore relative movement of the lobes of the model, loop generation techniques to generate alternative conformations of loops in the model, or Monte Carlo and/or molecular dynamics simulations.

Docking

A chemosensory receptor ligand, e.g., umami flavor entity was first docked into the active site of T1R1. Its modeled pose in the active site was selected by its ability to form productive van der Waals, ring stacking, hydrogen bonding, and/or salt bridging interactions with interacting residues within the active site of the Venus flytrap domain of T1R1.

A candidate for a chemosensory receptor ligand modifier, e.g., umami taste enhancer was then docked into the active site in the presence of the ligand, e.g., the umami flavor entity described in the previous paragraph. Its active pose and its candidacy as a potential chemosensory receptor ligand modifier, e.g., umami taste enhancer was based on its ability to form productive interactions in the form of van der Waals, ring stacking, hydrogen bonding, and/or salt bridging interactions with interacting residues described in the present invention, with additional residues of the T1R1 domain, and optionally with the chemosensory receptor ligand, e.g., the umami flavor entity placed in the active site as described above.

Candidate for Chemosensory Receptor Ligand Modifiers

A molecule was considered a candidate if it can be docked into the active site in the presence of a chemosensory receptor ligand, e.g., umami flavor entity, forming productive interactions with interacting residues described in the present invention. We defined two spaces within the active site: a first space occupied by a chemosensory receptor ligand, e.g., umami flavor entity, and a second space occupied by a chemosensory receptor ligand modifier, e.g., enhancer. Modeling and mutagenesis results established key residues that were considered to be likely to line these spaces for the chemosensory receptor ligand, e.g., umami flavor entity and chemosensory receptor ligand modifier, e.g., umami enhancers. In the context of our study, “residue lining the space” meant that the residue had backbone and/or side-chain atoms that were positioned so that they can potentially interact with atoms of the chemosensory receptor ligand, e.g., umami flavor entity (space #1) and/or chemosensory receptor ligand modifier, e.g., umami enhancer (space #2). While the chemosensory receptor ligand, e.g., umami flavor entity and chemosensory receptor ligand modifier, e.g., umami enhancer themselves cannot occupy the same space, their corresponding spaces may overlap due to the ability of residues to contact both the chemosensory receptor ligand, e.g., umami flavor entity and the chemosensory receptor ligand modifier, e.g., umami enhancer, due to protein flexibility, due to ligand flexibility, and due to the potential for multiple binding modes for a chemosensory receptor ligand, e.g., umami flavor entity or chemosensory receptor ligand modifier, e.g., umami enhancer. Information on important residues lining space #1 and space #2 came from modeling and docking and from site directed mutagenesis.

The hinge residues are considered to be associated with the first space (space #1). We have discovered that one of the spaces occupied by a chemosensory receptor ligand, e.g., umami flavor entity is partially lined by residues herein called hinge residues. Many Venus flytrap domains have been crystallized with agonists including mGluR1, mGluR2, and mGluR3 that show agonists forming interactions with homologous residues to those identified herein for T1R1. Many chemosensory receptor ligands, e.g., umami flavor entities docked to the model of T1R1 can be docked to this region. Our site directed mutagenesis also provides strong evidence to support the finding that hinge residues or residues spatially adjacent to it are key residues to the activation of a chemosensory receptor, e.g., T1R1 related receptor. Since chemosensory receptor ligands, e.g., umami flavor entity vary in size, there are additional residues lining this first space for larger residues where the list of these additional residues is dependent, partially on the size of the chemosensory receptor ligand, e.g., umami flavor entity.

Pincer residues are considered to be associated with the second space (space #2). Venus flytrap domains are known to transition from an “open” state to a “closed” state on agonist binding. The flytrap domain is comprised of two lobes commonly referred to in the literature as the upper lobe and lower lobe. In the “open” state the lobes are further apart, while in the closed state the lobes undergo a relative motion that brings the upper and lower lobe closer together. In addition to direct stabilization of the closed state of T1R1 by the agonist, our modeling study has demonstrated that there is additional stabilization of the closed state through interactions of residues on the upper lobe with corresponding residues on the lower lobe that are herein called the “pincer residues”. We have discovered that an interacting site, e.g., interacting space for a chemosensory receptor ligand modifier, e.g., umami enhancer is the space that is partially lined by these pincer residues, since additional interactions in this region can further stabilize the closed, agonized form of the Venus flytrap domain. Our site directed mutagenesis study also provides evidence to support the finding that pincer residues and residues spatially adjacent to them are key residues associated with modulation of chemosensory receptor ligand, e.g., enhancement activity of the ligand.

In determining whether or not residues brought into close proximity in the closed state directly contribute to stabilization of the closed state via interactions between the lobes, their proximity offers a meaningful target for the identification, design, and improvement of ligands to stabilize the closed state.

Procedural Definitions.

1. Docking

Docking is generally considered as the process of translating and rotating the candidate molecule relative to a chemosensory receptor, e.g., T1R1 structural model while simultaneously adjusting internal torsional angles of the candidate molecule to fit the candidate molecule into the active site of the chemosensory receptor, e.g., T1R1 structural model. Poses of the candidate molecule (positions, relative orientations, and internal torsions) are selected based on whether the molecule fits the active site, and whether the molecule can form productive van der Waals interactions, hydrogen bonds, ring stacking interactions, and salt bridge interactions with residues of the active site and with the chemosensory receptor ligand, e.g., umami flavor entity. Key residues can be identified. A candidate is considered more likely if it interacts with sets of residues in the active site as the hinge region, the near active site, the pincer residues, the charged residues identified as relevant for receptor ligand modifier interaction, and the totality of the active site. It is also considered more likely if it forms direct interactions with a chemosensory receptor ligand, e.g., a umami flavor entity.

2. Homology Modeling

Homology modeling is generally considered as the process of constructing a model of the Venus flytrap domain of a chemosensory receptor, e.g., T1R1 from its amino acid sequence and from the three dimensional coordinates of one or more homologous Venus flytrap domain proteins. Homology modeling may be performed using standard methods well-described in the literature and available in commercial software such as the Homology program or Modeler from the Accelrys Corporation. Models based on experimentally determined structures of open and closed forms, as well as animation of models using normal mode analysis, were used to define the pincer residues discussed above.

Exemplary Illustrations of Modeling Studies

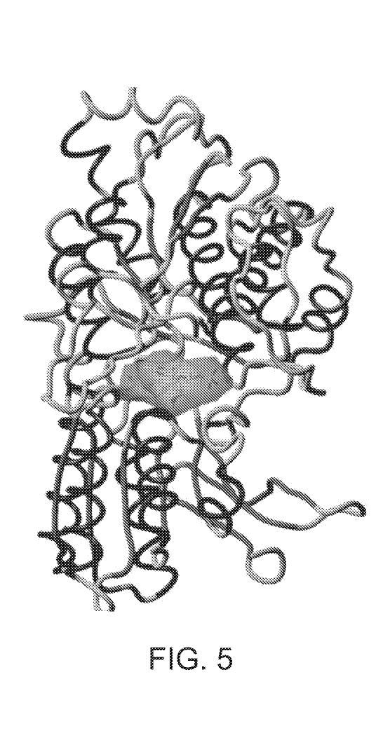

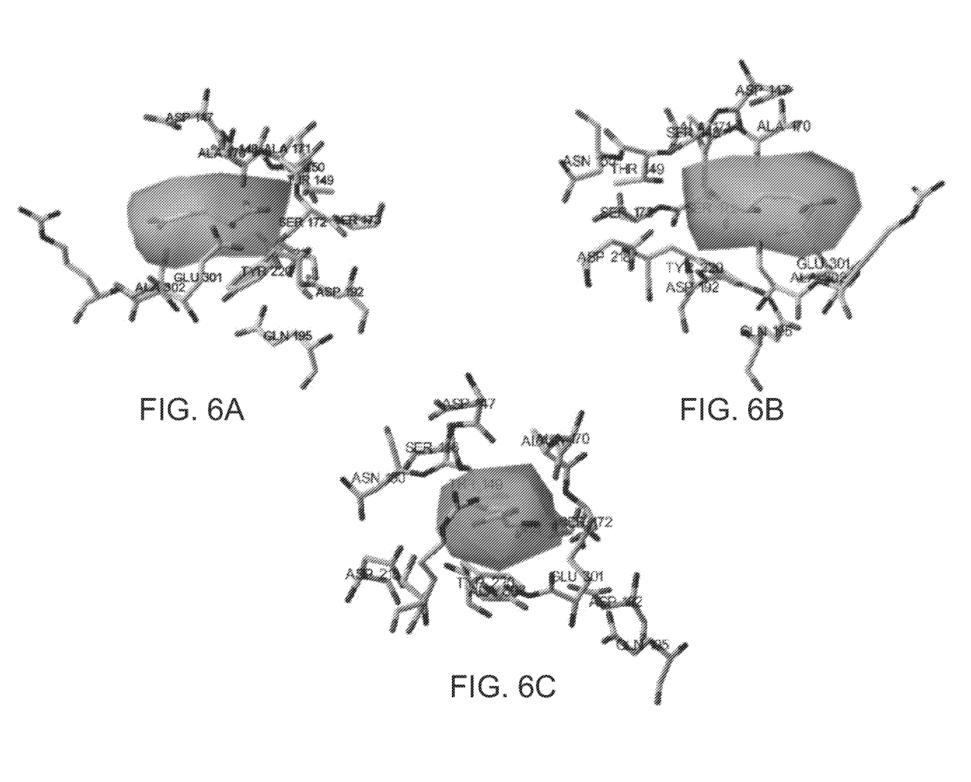

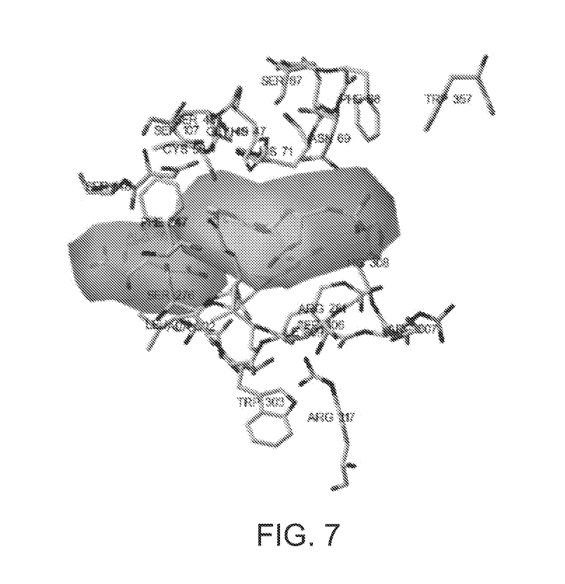

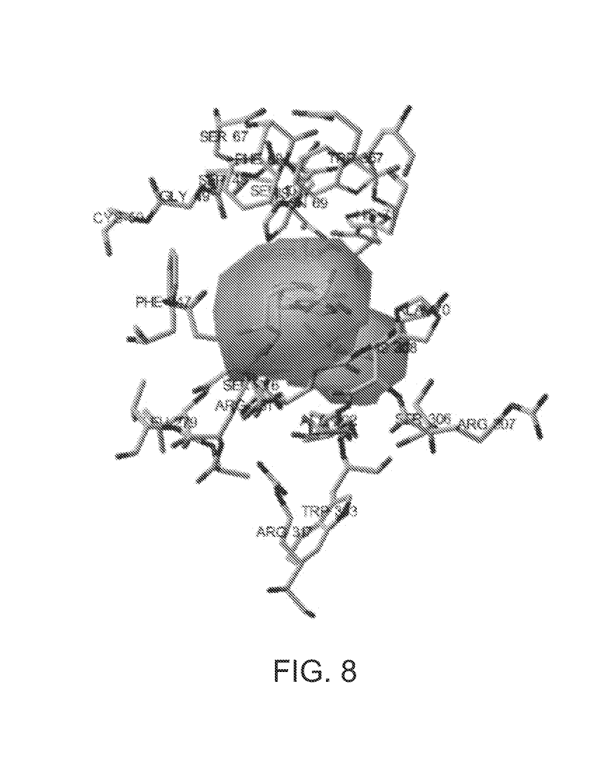

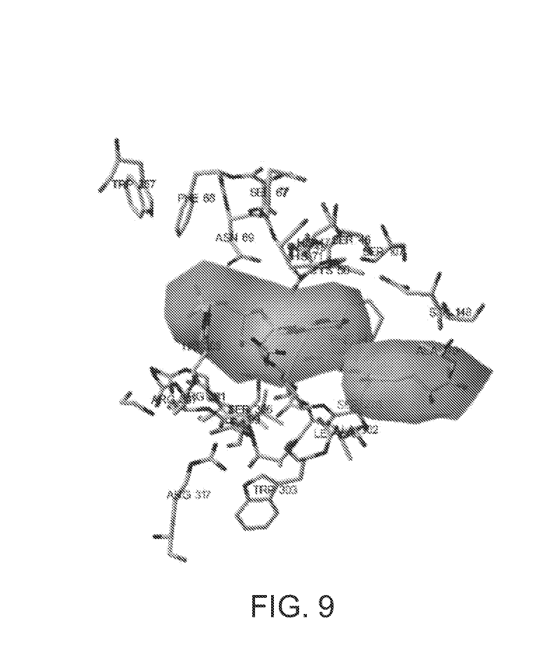

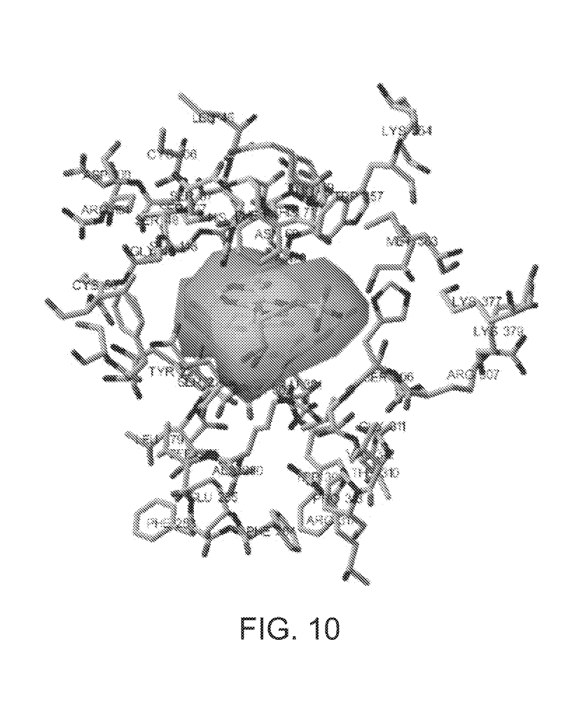

FIGS. 5 to 10 illustrate interacting spaces and residues associated with one of our molecular modeling studies.

### Experiment 2

### Mutagenesis Study for Identification of Chemosensory Receptor Ligand Modifier: Enhancer

In our previous patent applications (International Publication No. WO070104709), we described a method using sweet-umami chimeric receptors to map the binding sites of sweet and umami tastants. Our data demonstrated that a number of sweeteners, including sucrose, fructose, arspartame, neotame, D-tryptophan (D-Trp), Acesulfame K, saccharin and dulcin, all interact with the T1R2Venus flytrap domain (VFT), while the umami tastants, including L-glutamate (L-Glu), L-aspartate (L-Asp), and L-AP4 (2-amino-4-phosphonobutyrate), and the umami enhancers, including inosine-5′-monophosphate (IMP), and guanosine-5′-monophosphate (GMP), all interact with the T1R1 Venus flytrap domain.

In order to further define the interaction sites of umami stimuli, we performed site-directed mutagenesis on human T1R1 VFT. The mutagenesis was done using the routine PCR-based method. Human T1R2 mutants were transiently transfected into HEK293 cell together with the rat T1R3 wild type cDNA, and the transfected cells were characterized using an automated FLIPR machine or a calcium imaging system as described in our previous patent applications. In order to control for plasma membrane expression, protein folding and other factors that might contribute to changes in receptor activity, we used compound X (Senomyx) as a positive control. It is known from our previous data that compound X interacts with the human T1R1 transmembrane domain.

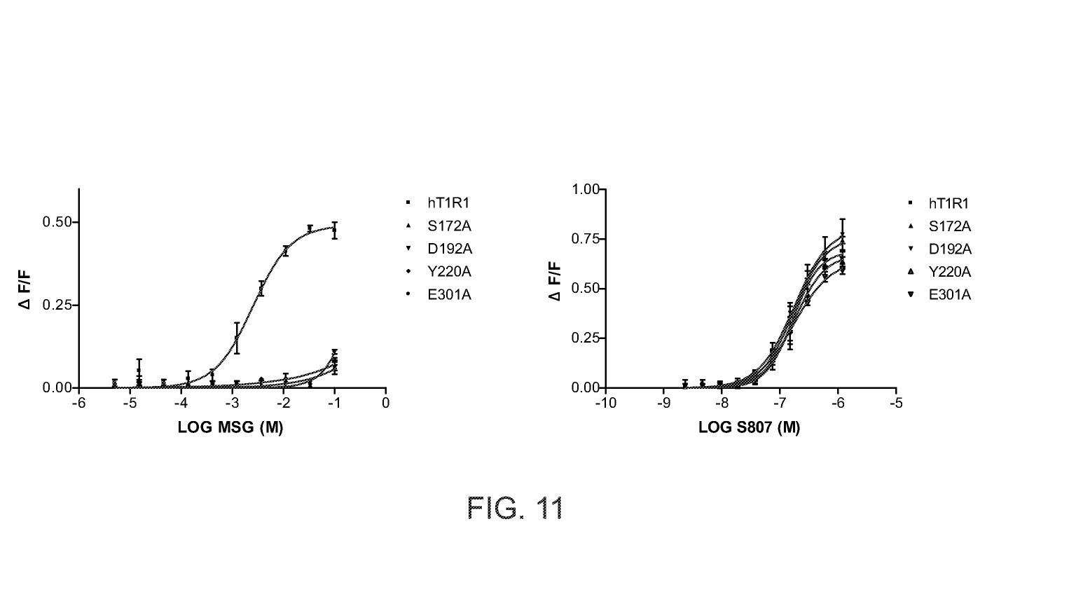

We generated and characterized more than 30 hT1R1 mutants, 4 (S172, D192, Y220, E301) of which elicited significant reduced activity against L-Glu, while the activity against the control compound X was not affected as shown in FIG. 11.

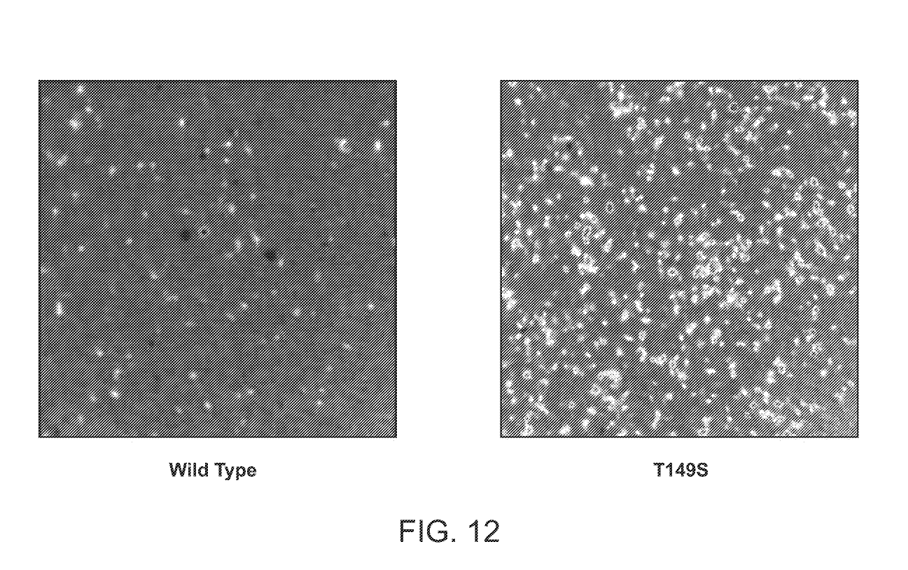

T149, when mutated to Ser, resulted in increased activity against L-Glu compared to the wild type. FIG. 12 shows activity of wild type hT1R1 and hT1R1 mutant T149S, responding to 0.25 mM of L-Glu. T149 is therefore another residue critical for interaction with L-Glu.

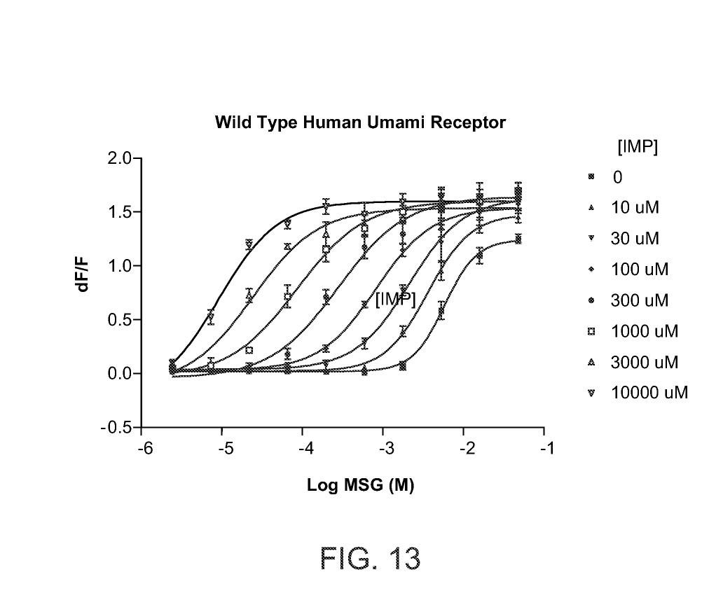

IMP is a natural enhancer of the umami receptor. As shown in FIG. 13, the wild type human umami receptor was strongly enhanced by IMP. 10 mM IMP can shift the dose response of L-Glu by hundreds of folds.

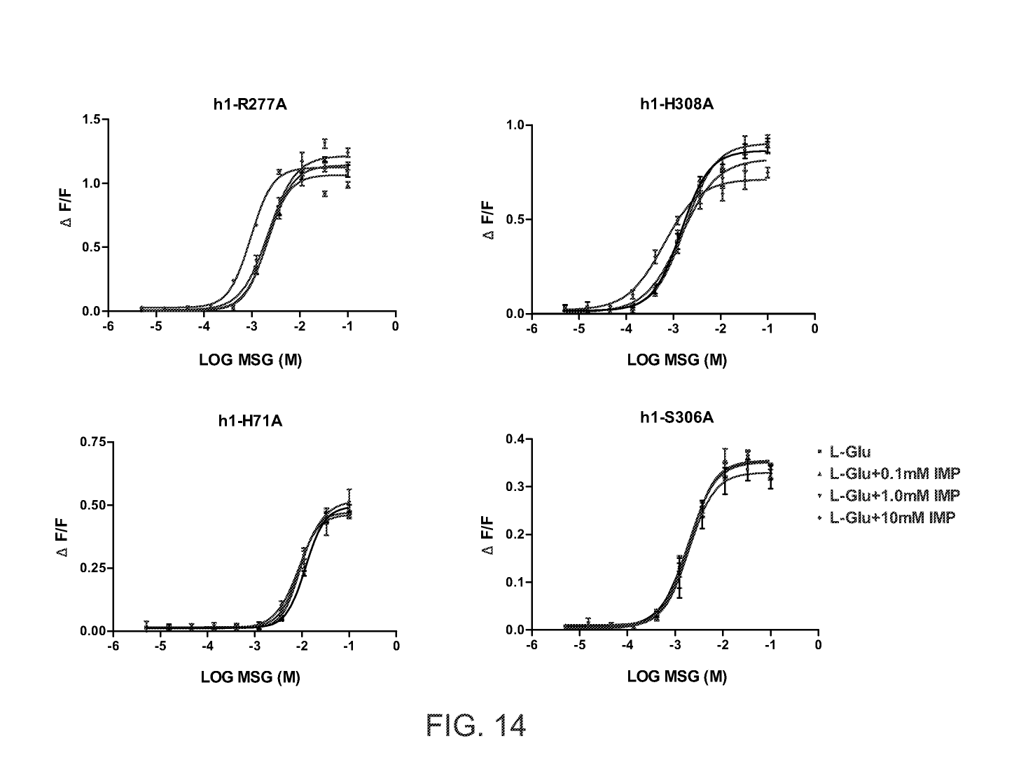

Based on the mutagenesis data, 4 residues (S306, H71, R277, H308) are critical for the IMP enhancement activity. As shown in FIG. 14, mutation in any of the 4 residues abolished the enhancement activity of IMP. The other natural umami enhancer, GMP, was similarly affected by these mutations.

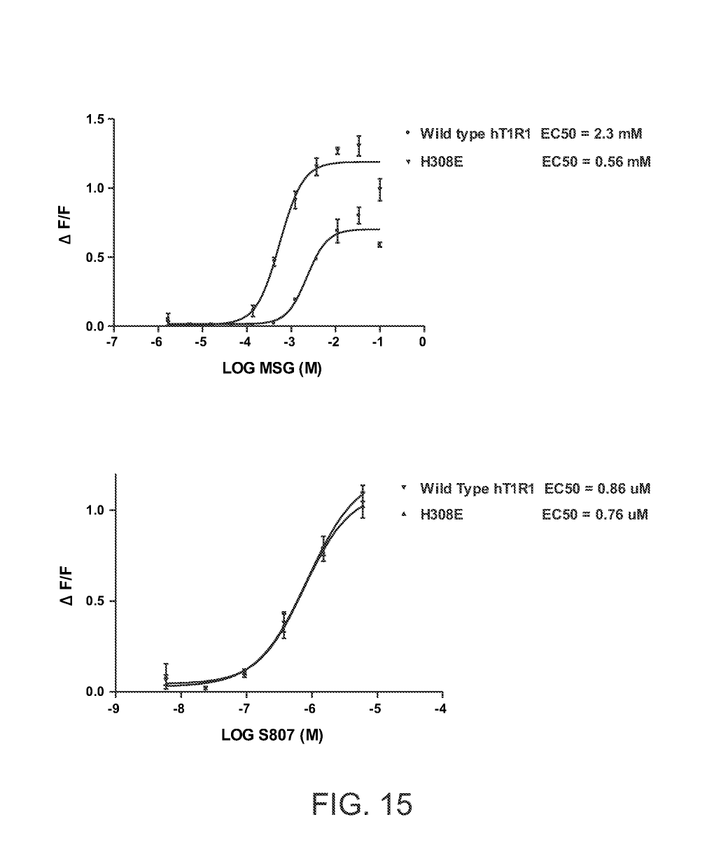

Based on our molecular model of hT1R1 VFT, we believed that mutagenesis of the pincer residues into a residue of opposite charge could stabilize the closed conformation, and result in increase activity compared to wild type hT1R1. One of such mutants, H308E, indeed showed increased activity, as shown in FIG. 15. The activity of the control compound X was not affected by the mutations, indicating the increased effect was not due to increased surface expression. This observation provided strong support for our hT1R1 VFT model.

The mutagenesis data for a number of umami flavor entities are summarized in the following tables. Based on the data, we concluded that 5 residues (S172, Y220, D192, E301, T149) are critical for interaction with L-glutamate and for interaction with umami enhancers IMP and GMP.

| | L-Glu Response | IMP Enhancement | GMP Enhancement |
|---|---|---|---|
| hT1R1 | (25 mM) | (10 mM) | (10 mM) |
| WT | ++ | ++ | ++ |
| S306A | + | − | − |
| H71A | + | − | − |
| R277A | ++ | − | − |
| H308A | ++ | − | − |
| H308E* | +++ | − | − |
| S172A | − | + | ND |
| Y220A | − | + | ND |
| D192A | − | ++ | ND |
| E301A | − | ++ | ND |
| T149S* | +++ | ++ | ++ |

*These mutants are more sensitive to the L-Glu than the wide type receptor.

### Experiment 3

### Chemical Synthesis of the Compounds of the Present Invention

### Example 1

### Sodium ((2R,3S,4R,5R)-3,4-dihydroxy-5-(6-hydroxy-2-(2-methoxybenzamido)-9H-purin-9-yl)tetrahydrofuran-2-yl)methyl phosphate

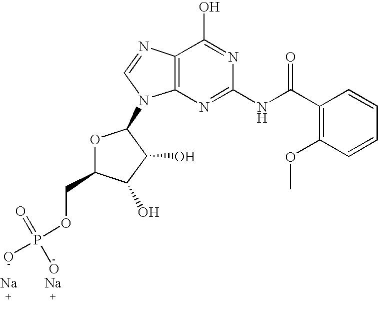

To a 50 ml flask was added N-(9-((2R,3R,4S,5R)-3,4-dihydroxy-5-(hydroxymethyl)tetrahydrofuran-2-yl)-6-hydroxy-9H-purin-2-yl)-2-methoxybenzamide (example 1a) (500 mg, 1.2 mmol) and POCl₃/PO(OEt)₃ (10 mL, 8 g in 100 mL PO(OEt)₃). The reaction mixture was stirred at 0° C. for 5 hours and then poured into aqueous NaHCO₃ (2 g in 20 mL H₂O). The solution was purified by preparative HPLC to give sodium ((2R,3S,4R,5R)-3,4-dihydroxy-5-(6-hydroxy-2-(2-methoxybenzamido)-9H-purin-9-yl)tetrahydrofuran-2-yl)methyl phosphate (0.13 g, yield 20%). ¹HNMR (400 MHz, DMSO-d₆): δ 8.36 (s, 1H), 7.84 (d, J=8.4 Hz, 1H), 7.47 (t, J1=7.6 Hz, J2=8.8 Hz, 1H), 7.09 (d, J=8.4 Hz, 1H), 6.67 (t, J=7.6 Hz, 1H), 5.95 (d, J=5.2 Hz, 1H), 4.61 (m, 1H), 4.41 (s, 1H), 4.29 (s, 1H), 4.20 (s, 2H), 4.02 (s, 3H).

### Example 1a

To a solution of N-(9-((2R,3R,4R,5R)-3,4-bis(trimethylsilyloxy)-5-((trimethylsilyloxy)methyl)tetrahydrofuran-2-yl)-6-hydroxy-9H-purin-2-yl)-2-methoxybenzamide (example 1b) (15 g) in Et₂O (400 mL) was added 10% TFA (50 mL, 4 mL TFA in 46 mL CH₂Cl₂). The reaction mixture was stirred at ambient temperature for 30 min. Upon completion, the reaction mixture was filtered, and the filter cake was washed with Et₂O (150 mL) to give pure N-(9-((2R,3R,4S,5R)-3,4-dihydroxy-5-(hydroxymethyl)tetrahydrofuran-2-yl)-6-hydroxy-9H-purin-2-yl)-2-methoxybenzamide (8.4 g, yield 84.8%). ¹HNMR (400 MHz,-DMSO-d₆): δ 12.2 (s, 1H), 11.35 (s, 1H), 8.26 (s, 1H), 7.76 (dd, J1=1.6 Hz, J2=7.6 Hz, 1H), 7.61 (t, J1=1.2 Hz, J2=8.2 Hz, 1H), 7.24 (d, 8.8 Hz, 1H), 7.12 (t, J=7.6 Hz, 1H), 5.81 (d, J=6 Hz, 1H), 4.5 (m, 1H), 4.13 (m, 1H), 3.96 (s, 3H), 3.91 (m, 1H), 3.63 (m, 1H), 3.54 (m, 1H).

### Example 1b

To a solution of (2R,3R,4S,5R)-2-(2-amino-6-hydroxy-9H-purin-9-yl)-5-(hydroxymethyl)tetrahydrofuran-3,4-diol (20 g, 0.07 mol) and pyridine (400 mL) in CH₂Cl₂ (1.5 L) was added dropwise TMSC1 (100 g) at 0° C. The mixture was stirred at 0° C. for 4 hours. 2-methoxybenzoyl chloride was added dropwise to the stirred solution at 0° C. and the reaction was stirred at room temperature overnight. The reaction mixture was slowly poured into ice water, and extracted with CH₂Cl₂ (1.5 L×3). The combined extracts was washed with water (1.5 L×4) and brine (1.2 L×3), dried over MgSO₄, filtered, concentrated in vacuo to give crude N-(9-((2R,3R,4R,5R)-3,4-bis(trimethylsilyloxy)-5-((trimethylsilyloxy)methyl)tetrahydrofuran-2-yl)-6-hydroxy-9H-purin-2-yl)-2-methoxybenzamide (32 g).

### Example 2

### Sodium ((2R,3S,4R,5R)-3,4-dihydroxy-5-(6-hydroxy-2-(4-methoxybenzamido)-9H-purin-9-yl)tetrahydrofuran-2-yl)methyl phosphate

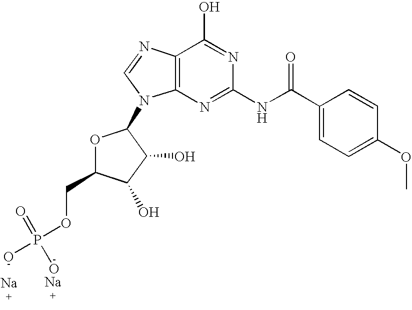

Prepared as described in Example 1 starting from (2R,3R,4S,5R)-2-(2-amino-6-hydroxy-9H-purin-9-yl)-5-(hydroxymethyl)tetrahydrofuran-3,4-diol and 4-methoxybenzoylchloride. MS (M+H, 542.1).

### Example 3

### Sodium ((2R,3S,4R,5R)-3,4-dihydroxy-5-(6-hydroxy-2-(2-fluorobenzamido)-9H-purin-9-yl)tetrahydrofuran-2-yl)methyl phosphate

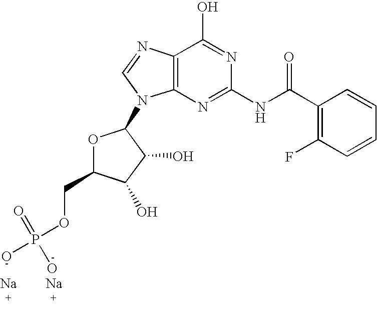

Prepared as described in Example 1 starting from (2R,3R,4S,5R)-2-(2-amino-6-hydroxy-9H-purin-9-yl)-5-(hydroxymethyl)tetrahydrofuran-3,4-diol and 2-fluorobenzoylchloride. MS (M+H, 530.1).

### Example 4

### Sodium ((2R,3S,4R,5R)-3,4-dihydroxy-5-(6-hydroxy-2-(3-methylbenzamido)-9H-purin-9-yl)tetrahydrofuran-2-yl)methyl phosphate

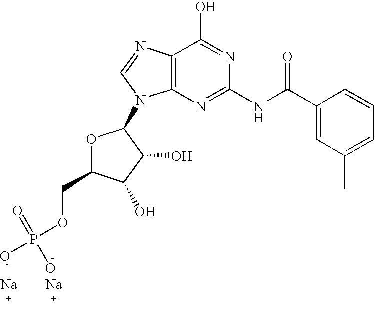

Prepared as described in Example 1 starting from (2R,3R,4S,5R)-2-(2-amino-6-hydroxy-9H-purin-9-yl)-5-(hydroxymethyl)tetrahydrofuran-3,4-diol and 3-methylbenzoylchloride. MS (M+H, 526.2).

### Example 5

### Sodium ((2R,3S,4R,5R)-3,4-dihydroxy-5-(6-hydroxy-2-(2-phenoxyacetamido)-9H-purin-9-yl)tetrahydrofuran-2-yl)methyl phosphate

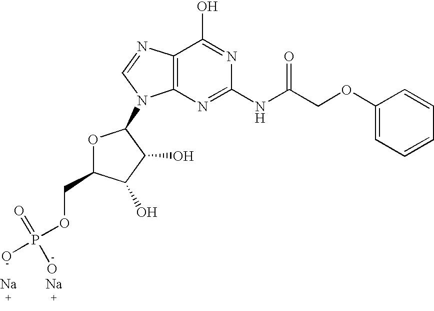

Prepared as described in Example 1 starting from (2R,3R,4S,5R)-2-(2-amino-6-hydroxy-9H-purin-9-yl)-5-(hydroxymethyl)tetrahydrofuran-3,4-diol and 2-phenoxyacetyl chloride. MS (M−2Na⁺, 495.9).

### Example 6

### Sodium ((2R,3S,4R,5R)-3,4-dihydroxy-5-(6-hydroxy-2-(2-thiophenamido)-9H-purin-9-yl)tetrahydrofuran-2-yl)methyl phosphate

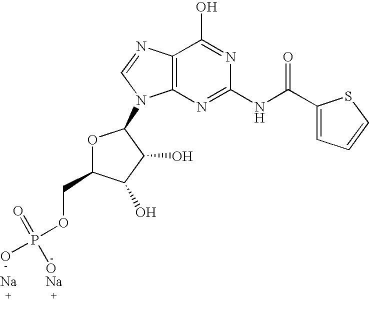

Prepared as described in Example 1 starting from (2R,3R,4S,5R)-2-(2-amino-6-hydroxy-9H-purin-9-yl)-5-(hydroxymethyl)tetrahydrofuran-3,4-diol and thiophene-2-carbonyl chloride. MS (M−PO₃²⁻2Na⁺, 392.3).

### Experiment 4

### Biological Assay

In Vitro hT1R1/hT1R3 Activation Assay:

An HEK293 cell line derivative (See e.g., Chandrashekar, et al., Cell (2000) 100: 703-711) which stably expresses Gα15 and hT1R1/hT1R3 under an inducible promoter (see e.g., International Publication No. WO 03/001876 A2) was used in association with identifying compounds with umami tasting properties.

Compounds were initially selected based on activity in the hT1R1/hT1R3-HEK293-Gα15 cell line. Activity was determined using an automated fluorometric imaging assay on a FLIPR instrument (Fluorometric Intensity Plate Reader, Molecular Devices, Sunnyvale, Calif.) (designated FLIPR assay). Cells from one clone (designated clone I-17) were seeded into 384-well plates (at approximately 48,000 cells per well) in a medium containing Dulbecco's modified Eagle's medium (DMEM) supplemented with GlutaMAX (Invitrogen, Carlsbad, Calif.), 10% dialyzed fetal bovine serum (Invitrogen, Carlsbad, Calif.), 100 Units/ml Penicillin G, 100 μg/ml Streptomycin (Invitrogen, Carlsbad, Calif.) and 60 pM mifepristone (to induce expression of hT1R1/hT1R3 (see e.g., International Publication No. WO 03/001876 A2).

I-17 cells were grown for 48 hours at 37° C. 1-17 cells were then loaded with the calcium dye Fluo-3AM (Molecular Probes, Eugene, Oreg.), 4 μM in a phosphate buffered saline (D-PBS) (Invitrogen, Carlsbad, Calif.), for 1.5 hours at room temperature. After replacement with 25 μl D-PBS, stimulation was performed in the FLIPR instrument and at room temperature by the addition of 25 μl D-PBS supplemented with different stimuli at concentrations corresponding to twice the desired final level.

Receptor activity was quantified by determining the maximal fluorescence increases (using a 480 nm excitation and 535 nm emission) after normalization to basal fluorescence intensity measured before stimulation. For dose-responses analysis, stimuli were presented in duplicates at 10 different concentrations ranging from 1.5 nM to 3 μM.

Activities were normalized to the response obtained with 60 mM monosodium glutamate, a concentration that elicits maximum receptor response. EC₅₀s (concentration of compound that causes 50% activation of receptor) were determined using a non-linear regression algorithm, where the Hill slope, bottom asymptotes and top asymptotes were allow to vary. Identical results were obtained when analyzing the dose-response data using commercially available software for non-linear regression analysis such as GraphPad PRISM (San Diego, Calif.).

In order to determine the dependency of hT1R1/hT1R3 for the cell response to different stimuli, selected compounds were subjected to a similar analysis on I-17 cells that had not been induced for receptor expression with mifepristone (designated as un-induced I-17 cells). The un-induced I-17 cells do not show any functional response in the FLIPR assay to monosodium glutamate or other umami-tasting substances. Compounds were presented to un-induced umami cells at 10 μM—or three times the maximum stimulation used in the dose-response analysis. Compounds exemplified in this example do not show any functional response when using un-induced umami cells in the FLIPR assay.

Experiments were also conducted to determine if test compounds can enhance the effect of monosodium glutamate on hT1R1/hT1R3 activity. In this assay, increasing concentrations of monosodium glutamate (ranging from 12 μM to 81 mM) were presented, in duplicates, in the presence or absence of a fixed concentration of the test compound. Typical compound concentrations tested were 100 μM, 30 μM, 10 μM, 3 μM, 1 μM, 0.3 μM, 0.1 μM and 0.03 μM. The relative efficacy of compounds at enhancing the receptor was determined by the calculating the magnitude of a shift in the EC₅₀ for monosodium glutamate.

Enhancement was defined as a ratio (EC₅₀R) corresponding to the EC₅₀ of monosodium glutamate, determined in the absence of the test compound, divided by the EC₅₀ of monosodium glutamate, determined in the presence of the test compound. In some embodiments, compounds have an EC₅₀R between about 0.7 and about 100. In other embodiments, compounds have an EC₅₀R between about 1.25 and about 75. In still other embodiments, compounds have an EC₅₀R between about 1.50 and about 60. Assay results for compounds are disclosed in the table below.

EC₅₀ ratio

| Compound | 3 μM | 10 μm | 30 μM | 100 μM |
|---|---|---|---|---|
| 1 | | | 1.67 | 12.91 |
| 2 | | | 1.46 | 0.93 |
| 3 | | | 1.12 | 0.86 |
| 4 | | | 1.07 | 1.19 |
| 5 | | | 1.21 | 2.22 |
| 6 | | | 1.12 | 1.07 |
| 7 | | | 0.96 | 0.98 |
| 8 | | | 1.19 | 0.99 |
| 9 | | | 4.27 | 17.04 |
| 10 | | | 3.34 | 17.00 |
| 11 | | | 2.48 | 8.81 |
| 12 | | | 1.95 | 6.39 |
| 13 | | | 1.81 | 5.50 |
| 14 | | | 2.12 | 6.44 |
| 15 | | | 1.92 | 6.15 |
| 16 | | | 2.41 | 7.68 |
| 17 | | | 2.56 | 8.41 |
| 18 | | | 1.80 | 5.62 |
| 19 | | | 1.17 | 1.33 |
| 20 | | | 1.20 | 1.87 |
| 21 | | | 1.30 | 1.75 |
| 22 | | | 1.16 | 1.73 |
| 23 | | | 4.58 | 18.12 |
| 24 | | | 1.32 | 2.49 |
| 25 | | | 1.63 | 2.73 |
| 26 | | | 1.13 | 1.50 |
| 27 | | | 0.99 | 1.19 |
| 28 | | | 1.17 | 2.45 |
| 29 | | | 1.23 | 1.65 |
| 30 | | | 1.08 | 1.61 |
| 31 | | | 1.37 | 2.87 |
| 32 | | | 1.83 | 4.05 |
| 33 | | | 1.15 | 1.24 |
| 34 | | | 1.26 | 1.85 |
| 35 | | | 1.30 | 1.83 |
| 36 | | | 1.75 | 2.95 |
| 37 | | | 28.48 | |
| 38 | | 1.78 | 4.62 | 23.82 |
| 39 | | 4.65 | 9.80 | 31.12 |
| 40 | | | 14.02 | 46.38 |
| 41 | | 4.45 | 8.80 | 35.71 |
| 42 | | | 4.75 | 13.43 |
| 43 | | 2.51 | 4.50 | |
| 44 | | | 2.16 | 4.44 |
| 45 | | | 1.14 | 1.95 |
| 46 | | | 6.23 | 20.76 |
| 47 | | | 1.26 | 1.58 |
| 48 | | | 1.01 | 1.25 |
| 49 | | | 1.45 | 1.64 |
| 50 | | | 1.00 | 1.15 |
| 51 | | | 1.07 | 1.10 |
| 52 | | | 1.14 | 1.29 |
| 53 | | | 1.17 | 2.71 |
| 54 | | | 0.96 | 1.35 |
| 55 | | | 1.07 | 1.23 |
| 56 | | | 1.29 | 2.42 |
| 57 | | | 1.09 | 1.30 |
| 58 | | | 1.03 | 1.16 |
| 59 | | | 1.82 | 4.61 |
| 60 | | | 1.18 | 1.74 |
| 61 | | | 1.14 | 1.53 |
| 62 | | | 1.03 | 1.28 |
| 63 | | | 1.34 | 1.39 |
| 64 | | | 1.18 | 1.25 |
| 65 | | | 1.15 | 1.02 |
| 66 | | | 1.81 | 1.18 |
| 67 | | | 1.22 | 1.25 |
| 68 | | | 1.58 | 2.82 |
| 69 | | | 1.49 | 4.23 |
| 70 | | | 4.75 | 16.29 |
| 71 | | | 0.94 | 1.53 |
| 72 | | | 9.48 | |
| 73 | | | 1.54 | 3.24 |
| 74 | | | 1.45 | 2.20 |
| 75 | | | 1.00 | 3.02 |
| 75 | | | 4.29 | 10.36 |
| 77 | | | 4.21 | 15.76 |
| 78 | | | 2.35 | 8.46 |
| 79 | | | 4.40 | 25.35 |
| 80 | | | 1.12 | 1.38 |
| 81 | | | 0.87 | 1.04 |
| 82 | | | 1.25 | 1.39 |
| 83 | | | 1.00 | 1.39 |
| 84 | | | 1.20 | 1.72 |
| 85 | | | 1.39 | 1.55 |
| 86 | | | 1.05 | 1.35 |
| 87 | | | 1.14 | 1.22 |
| 88 | | | 1.34 | 1.48 |
| 89 | | | 1.54 | 2.64 |
| 90 | | | 10.85 | 18.65 |
| 91 | | | 1.10 | 1.42 |
| 92 | | | 1.33 | 2.26 |
| 93 | | | 3.60 | 3.28 |
| 94 | | | 1.38 | 2.34 |
| 95 | | | 1.01 | 1.30 |
| 96 | | | 1.28 | 2.26 |
| 97 | | | 1.09 | 1.27 |
| 98 | | | 17.25 | 22.40 |
| 99 | | | 3.00 | 11.62 |
| 100 | | | 1.01 | 1.37 |
| 101 | | | 2.95 | 6.07 |
| 102 | | | 1.45 | 2.92 |
| 103 | | | 2.71 | 3.72 |
| 104 | | | 3.14 | 8.79 |
| 105 | | | 3.58 | 8.12 |
| 106 | | | 4.93 | 28.13 |
| 107 | | | 1.72 | 4.21 |
| 108 | | | 2.23 | 6.71 |
| 109 | | | 4.00 | 20.58 |
| 110 | | | 1.43 | |
| 111 | | | 5.64 | 50.51 |
| 112 | | | 1.85 | 4.18 |
| 113 | | | 2.51 | 4.63 |
| 114 | | | 7.63 | 27.55 |
| 115 | | | 5.09 | 10.88 |
| 116 | | | 3.60 | 9.51 |
| 117 | | | 4.85 | |
| 118 | 3.20 | 11.29 | 18.92 | |
| 119 | | | | 10.81 |
| 120 | | 1.77 | 3.54 | 24.91 |
| 121 | | | 3.34 | 15.70 |
| 122 | | | 10.17 | 40.86 |
| 123 | 2.97 | 16.01 | | |
| 124 | 16.76 | | | |
| 125 | | | 7.83 | 24.57 |
| 126 | | | 19.14 | 46.05 |
| 127 | | | 3.13 | 10.23 |
| 128 | | | 1.55 | 2.26 |
| 129 | | | 1.67 | 12.91 |
| 130 | | | 1.46 | 0.93 |
| 131 | | | 1.12 | 0.86 |
| 132 | | | 1.07 | 1.19 |
| 133 | | | 1.21 | 2.22 |
| 134 | | | 1.12 | 1.07 |
| 135 | | | 0.96 | 0.98 |
| 136 | | | 1.19 | 0.99 |

1. A method of screening for a candidate of a chemosensory receptor ligand modifier comprising
determining whether a test entity is suitable to interact with a chemosensory receptor via a first interacting site within the Venus flytrap domain of the chemosensory receptor.

2. The method of claim 1, wherein the first interacting site of the Venus flytrap domain of the chemosensory receptor includes one or more interacting residues of the Venus flytrap domain of the chemosensory receptor.

3. The method of claim 1, wherein the first interacting site of the Venus flytrap domain of the chemosensory receptor includes one or more interacting spaces of the Venus flytrap domain of the chemosensory receptor.

4. The method of claim 1, wherein the first interacting site of the Venus flytrap domain includes an interacting space identified based on one or more interacting residues.

5. The method of claim 1, wherein the first interacting site of the Venus flytrap domain of the chemosensory receptor includes one or more interacting residues, which are identified based on mutagenesis analysis of the Venus flytrap domain.

6. The method of claim 1, wherein the first interacting site of the Venus flytrap domain is identified based on computer modeling, X-ray crystallography, or a combination thereof.

7. The method of claim 1, wherein the first interacting site of the Venus flytrap domain is identified based on one or more known chemosensory receptor ligands.

8. The method of claim 1, wherein the first interacting site of the Venus flytrap domain is identified based on one or more known chemosensory receptor ligand modifiers.

9. The method of claim 1, wherein the first interacting site of the Venus flytrap domain is identified based on a predetermined chemosensory receptor ligand.

10. The method of claim 1, wherein the first interacting site of the Venus flytrap domain is predetermined.

11. The method of claim 1, wherein the first interacting site of the Venus flytrap domain is in the T1R1 Venus flytrap domain.

12. The method of claim 1, wherein the first interacting site of the Venus flytrap domain is in the T1R1 Venus flytrap domain and is identified in the presence of T1R3 Venus flytrap domain.

13. The method of claim 1, wherein the determination is carried out in silico.

14. A method of screening for a candidate of a chemosensory receptor ligand modifier comprising
determining whether a test entity is suitable to interact with a chemosensory receptor via a first interacting site within the Venus flytrap domain of the chemosensory receptor,
wherein the first interacting site is identified in light of a second interacting site identified based on the interaction between a chemosensory receptor ligand and the chemosensory receptor.

15. The method of claim 14, wherein the first and second interacting site are in the T1R1 Venus flytrap domain.

16. The method of claim 14, wherein the first and second interacting site are in the T1R1 Venus flytrap domain and identified in the presence of T1R3 Venus flytrap domain.

17. The method of claim 1, wherein the first interacting site includes an interacting residue selected from the group consisting of amino acid D147, S148, T149, N150, A170, A171, S172, S173, D192, N195, D218, Y220, S276, R277, E301, and A302 of a human T1R1 and a combination thereof.

18. The method of claim 1, wherein the first interacting site includes an interacting residue selected from the group consisting of amino acid H47, S48, G49, C50, S67, F68, N69, E70, H71, S107, D147, S148, A170, F247, S276, R277, Q278, L279, A280, R281, V282, A302, W303, S306, R307, H308, I309, G311, R317, and W357 of a human T1R1 and a combination thereof.

19. The method of claim 1, wherein the first interacting site includes an interacting residue selected from the group consisting of amino acid L46, H47, S48, G49, C50, L51, S67, F68, N69, E70, H71, C106, S107, D108, D147, S148, R151, Y169, A170, Y220, F247, S248, S275, S276, R277, Q278, L279, A280, R281, V282, F283, F284, E285, E301, A302, W303, S306, R307, H308, I309, T310, G311, V312, P313, R317, K354, W357, K377, K379, M383, and S385 of a human T1R1 and a combination thereof.

20. The method of claim 1, wherein the first interacting site includes an interacting residue selected from the group consisting of amino acid L46, H47, S48, G49, C50, L51, S67, F68, N69, E70, H71, C106, S107, D108, D147, S148, T149, N150, R151, Y169, A170, A171, S172, S173, D192, N195, D218, Y220, S276, R277, E301, A302, F247, S248, S275, S276, R277, Q278, L279, A280, R281, V282, F283, F284, E285, E301, A302, W303, S306, R307, H308, I309, T310, G311, V312, P313, R317, K354, W357, K377, K379, M383, and S385 of a human T1R1 and a combination thereof.

21. The method of claim 1, wherein the first interacting site includes a group of interacting residues selected from the group consisting of 1) D147, S148, T149, N150, A170, A171, S172, S173, D192, N195, D218, Y220, S276, R277, E301, and A302 of a human T1R1, 2) H47, S48, G49, C50, S67, F68, N69, E70, H71, S107, D147, S148, A170, F247, S276, R277, Q278, L279, A280, R281, V282, A302, W303, S306, R307, H308, I309, G311, R317, and W357 of a human T1R1, 3) L46, H47, S48, G49, C50, L51, S67, F68, N69, E70, H71, C106, S107, D108, D147, S148, R151, Y169, A170, Y220, F247, S248, S275, S276, R277, Q278, L279, A280, R281, V282, F283, F284, E285, E301, A302, W303, S306, R307, H308, I309, T310, G311, V312, P313, R317, K354, W357, K377, K379, M383, and S385 of a human T1R1, 4) L46, H47, S48, G49, C50, L51, S67, F68, N69, E70, H71, C106, S107, D108, D147, S148, T149, N150, R151, Y169, A170, A171, S172, S173, D192, N195, D218, Y220, S276, R277, E301, A302, F247, S248, S275, S276, R277, Q278, L279, A280, R281, V282, F283, F284, E285, E301, A302, W303, S306, R307, H308, I309, T310, G311, V312, P313, R317, K354, W357, K377, K379, M383, and S385 of a human T1R1, 5) S172, Y220, D192, E301, and T149 of a human T1R1, and 6) a combination thereof.

22. The method of claim 1, wherein the first interacting site includes an interacting residue selected from the group consisting of amino acid L46, H47, S48, G49, C50, L51, S67, F68, N69, E70, H71, C106, S107, D108, D147, S148, R151, Y169, A170, Y220, F247, S248, S275, S276, R277, Q278, L279, A280, R281, V282, F283, F284, E285, E301, A302, W303, S306, R307, H308, I309, T310, G311, V312, P313, R317, K354, W357, K377, K379, M383, and S385 of a human T1R1 and a combination thereof and wherein a test entity suitable to interact with the first interacting site of the chemosensory receptor is indicative of a candidate of a chemosensory receptor ligand enhancer.

23. The method of claim 1, wherein the test entity is a designed compound structure.

24. The method of claim 1, wherein the chemosensory receptor ligand is a umami flavor entity selected from the group consisting of L-amino acids, monosodium glutamate, L-AP4, and succinate.

25. A method of screening for a candidate of a chemosensory receptor modifier comprising
determining whether a test entity is suitable to interact with a chemosensory receptor via an interacting site within the Venus flytrap domain of the chemosensory receptor,
wherein the interacting site includes an interacting residue selected from the group consisting of D147, S148, T149, N150, A170, A171, S172, S173, D192, N195, D218, Y220, S276, R277, E301, and A302 of a human T1R1 and a combination thereof, and
wherein a test entity suitable to interact with the interacting site of the chemosensory receptor is indicative of a candidate of a chemosensory receptor modifier.

26. The method of claim 25, wherein the interacting site is in the T1R1 Venus flytrap domain.

27. The method of claim 25, wherein the interacting site is in the T1R1 Venus flytrap domain and identified in the presence of T1R3 Venus flytrap domain.

28. The method of claim 25, wherein the determination is carried out in silico.

29. The method of claim 25, wherein the test entity is a designed compound structure.

30. A chemosensory receptor ligand modifier identified by the method of claim 1.

31. A chemosensory receptor modifier identified by the method of claim 25.

32. A chemosensory receptor ligand enhancer identified by the method of claim 1 and having a structural formula selected from the group consisting of
1) structural Formula (I) 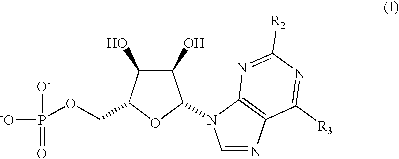
or a salt, hydrate or solvate thereof, wherein:
R₂ is hydrogen, —NR₄R₅ or —NR₄C(O)R₅;
R₄ and R₅ are independently hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl or substituted heteroarylalkyl;
R₃ is hydroxyl, —NR₆R₇, —NR₆C(O)R₇ or —S(O)ₐR₆;
R₆ and R₇ are independently hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl or substituted heteroarylalkyl; and
a is 0, 1 or 2;
provided that when R₂ is hydrogen then R₃ is not hydroxyl; and
when R₂ is —NH₂ then R₃ is not hydroxyl,
2) structural Formula (II) 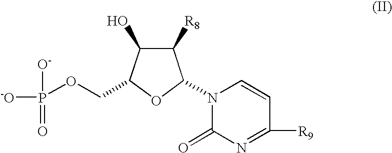
or a salt, hydrate or solvate thereof, wherein:
R₈ is hydrogen or hydroxyl;
R₉ is —NR₁₀C(O)R₁₁;
R₁₀ is hydrogen, substituted alkyl, aryl, substituted aryl, arylalkyl, substituted arylalkyl, heteroalkyl, substituted heteroalkyl, heteroaryl, substituted heteroaryl, heteroarylalkyl or substituted heteroarylalkyl; and
R₁₁ is hydrogen, (C₁-C₁₀)alkyl, substituted alkyl, aryl, substituted aryl, arylalkyl, substituted arylalkyl, heteroalkyl, substituted heteroalkyl, heteroaryl, substituted heteroaryl, heteroarylalkyl or substituted heteroarylalkyl,
3) structural Formula (III): 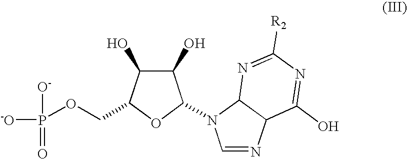
or a salt, hydrate or solvate thereof, wherein:
R₂ is hydrogen, —NR₄R₅ or —NR₄C(O)R₅; and
R₄ and R₅ are independently hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl or substituted heteroarylalkyl, and
4) structural Formula (IV): 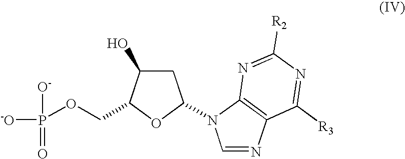
or a salt, hydrate or solvate thereof, wherein:
R₂ is hydrogen, —NR₄R₅ or —NR₄C(O)R₅;
R₄ and R₅ are independently hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl or substituted heteroarylalkyl; and
R₃ is hydroxyl, —NR₆R₇ or —NR₆C(O)R₇.

33. A method of modulating the activity of a chemosensory receptor ligand comprising contacting a chemosensory receptor ligand modifier with a cell containing T1R1 Venus flytrap domain in the presence of a chemosensory receptor ligand, wherein the chemosensory receptor ligand modifier interacts with an interacting site of the chemosensory receptor.

34. The method of claim 33, wherein the interacting site of the chemosensory receptor includes an interacting residue selected from the group consisting D147, S148, T149, N150, A170, A171, S172, S173, D192, N195, D218, Y220, S276, R277, E301, and A302 of a human T1R1 and a combination thereof.

35. The method of claim 33, wherein the interacting site of the chemosensory receptor includes an interacting residue selected from the group consisting of amino acid H47, S48, G49, C50, S67, F68, N69, E70, H71, S107, D147, S148, A170, F247, S276, R277, Q278, L279, A280, R281, V282, A302, W303, S306, R307, H308, I309, G311, R317, and W357 of a human T1R1 and a combination thereof.

36. The method of claim 33, wherein the interacting site of the chemosensory receptor includes an interacting residue selected from the group consisting of amino acid L46, H47, S48, G49, C50, L51, S67, F68, N69, E70, H71, C106, S107, D108, D147, S148, R151, Y169, A170, Y220, F247, S248, S275, S276, R277, Q278, L279, A280, R281, V282, F283, F284, E285, E301, A302, W303, S306, R307, H308, I309, T310, G311, V312, P313, R317, K354, W357, K377, K379, M383, and S385 of a human T1R1 and a combination thereof.

37. The method of claim 33, wherein the interacting site of the chemosensory receptor includes an interacting residue selected from the group consisting of amino acid L46, H47, S48, G49, C50, L51, S67, F68, N69, E70, H71, C106, S107, D108, D147, S148, T149, N150, R151, Y169, A170, A171, S172, S173, D192, N195, D218, Y220, S276, R277, E301, A302, F247, S248, S275, S276, R277, Q278, L279, A280, R281, V282, F283, F284, E285, E301, A302, W303, S306, R307, H308, I309, T310, G311, V312, P313, R317, K354, W357, K377, K379, M383, and S385 of a human T1R1 and a combination thereof.

38. The method of claim 33, wherein the interacting site of the chemosensory receptor includes a group of interacting residues selected from the group consisting of 1) D147, S148, T149, N150, A170, A171, S172, S173, D192, N195, D218, Y220, S276, R277, E301, and A302 of a human T1R1, 2) H47, S48, G49, C50, S67, F68, N69, E70, H71, S107, D147, S148, A170, F247, S276, R277, Q278, L279, A280, R281, V282, A302, W303, S306, R307, H308, I309, G311, R317, and W357 of a human T1R1, 3) L46, H47, S48, G49, C50, L51, S67, F68, N69, E70, H71, C106, S107, D108, D147, S148, R151, Y169, A170, Y220, F247, S248, S275, S276, R277, Q278, L279, A280, R281, V282, F283, F284, E285, E301, A302, W303, S306, R307, H308, I309, T310, G311, V312, P313, R317, K354, W357, K377, K379, M383, and S385 of a human T1R1, 4) L46, H47, S48, G49, C50, L51, S67, F68, N69, E70, H71, C106, S107, D108, D147, S148, T149, N150, R151, Y169, A170, A171, S172, S173, D192, N195, D218, Y220, S276, R277, E301, A302, F247, S248, S275, S276, R277, Q278, L279, A280, R281, V282, F283, F284, E285, E301, A302, W303, S306, R307, H308, I309, T310, G311, V312, P313, R317, K354, W357, K377, K379, M383, and S385 of a human T1R1, 5) S172, Y220, D192, E301, and T149 of a human T1R1, and 6) a combination thereof.

39. The method of claim 33, wherein the interacting site of the chemosensory receptor includes an interacting residue selected from the group consisting of amino acid L46, H47, S48, G49, C50, L51, S67, F68, N69, E70, H71, C106, S107, D108, D147, S148, R151, Y169, A170, Y220, F247, S248, S275, S276, R277, Q278, L279, A280, R281, V282, F283, F284, E285, E301, A302, W303, S306, R307, H308, I309, T310, G311, V312, P313, R317, K354, W357, K377, K379, M383, and S385 of a human T1R1 and a combination thereof and wherein the chemosensory receptor ligand modifier enhances the activity of a chemosensory receptor ligand.

40. The method of claim 39, wherein the chemosensory receptor ligand is a L-amino acid.

41. The method of claim 33, wherein the chemosensory receptor ligand modifier stabilizes one or more positively charged residues located on a lobe of a chemosensory receptor.

42. The method of claim 33, wherein the chemosensory receptor ligand modifier is a chemosensory receptor ligand enhancer and has a structural formula selected from the group consisting of
1) structural Formula (I) 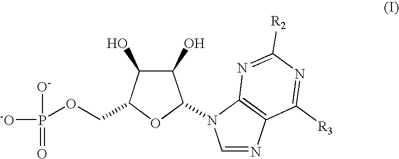
or a salt, hydrate or solvate thereof, wherein:
R₂ is hydrogen, —NR₄R₅ or —NR₄C(O)R₅;
R₄ and R₅ are independently hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl or substituted heteroarylalkyl;
R₃ is hydroxyl, —NR₆R₇, —NR₆C(O)R₇ or —S(O)ₐR₆;
R₆ and R₇ are independently hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl or substituted heteroarylalkyl; and
a is 0, 1 or 2;
provided that when R₂ is hydrogen then R₃ is not hydroxyl; and
when R₂ is —NH₂ then R₃ is not hydroxyl,
2) structural Formula (II) 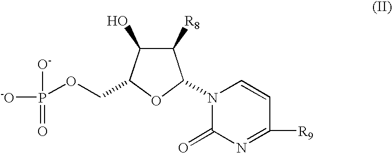
or a salt, hydrate or solvate thereof, wherein:
R₈ is hydrogen or hydroxyl;
R₉ is —NR₁₀C(O)R₁₁;
R₁₀ is hydrogen, substituted alkyl, aryl, substituted aryl, arylalkyl, substituted arylalkyl, heteroalkyl, substituted heteroalkyl, heteroaryl, substituted heteroaryl, heteroarylalkyl or substituted heteroarylalkyl; and
R₁₁ is hydrogen, (C₁-C₁₀)alkyl, substituted alkyl, aryl, substituted aryl, arylalkyl, substituted arylalkyl, heteroalkyl, substituted heteroalkyl, heteroaryl, substituted heteroaryl, heteroarylalkyl or substituted heteroarylalkyl,
3) structural Formula (III): 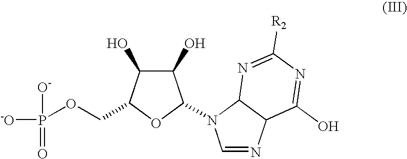
or a salt, hydrate or solvate thereof, wherein:
R₂ is hydrogen, —NR₄R₅ or —NR₄C(O)R₅; and
R₄ and R₅ are independently hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl or substituted heteroarylalkyl, and
4) structural Formula (IV): 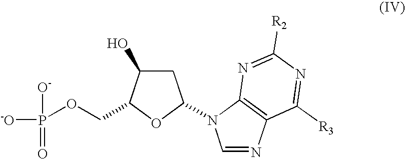
or a salt, hydrate or solvate thereof, wherein:
R₂ is hydrogen, —NR₄R₅ or —NR₄C(O)R₅;
R₄ and R₅ are independently hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl or substituted heteroarylalkyl; and
R₃ is hydroxyl, —NR₆R₇ or —NR₆C(O)R₇.

43. The method of claim 33, wherein the cell contains T1R1 Venus flytrap domain within a GPCR pathway.

44. The method of claim 33, wherein the chemosensory receptor ligand modifier is provided in a comestible composition.

45. The method of claim 33, wherein the chemosensory receptor ligand modifier is provided in a medicinal composition.

46. The method of claim 33, wherein the chemosensory receptor ligand modifier is provided in a food or beverage product.

47. A chemosensory receptor ligand modifier, wherein in the presence of a chemosensory receptor ligand it interacts with T1R1 Venus flytrap domain via at least three interacting residues selected from the group consisting of L46, H47, S48, G49, C50, L51, S67, F68, N69, E70, H71, C106, S107, D108, D147, S148, T149, N150, R151, Y169, A170, A171, S172, S173, D192, N195, D218, Y220, S276, R277, E301, A302, F247, S248, S275, S276, R277, Q278, L279, A280, R281, V282, F283, F284, E285, E301, A302, W303, S306, R307, H308, I309, T310, G311, V312, P313, R317, K354, W357, K377, K379, M383, and S385 of a human T1R1.

48. The chemosensory receptor ligand modifier of claim 47, wherein it interacts with T1R1 Venus flytrap domain via a group of amino acids selected from the group consisting of 1) D147, S148, T149, N150, A170, A171, S172, S173, D192, N195, D218, Y220, S276, R277, E301, and A302 of a human T1R1, 2) H47, S48, G49, C50, S67, F68, N69, E70, H71, S107, D147, S148, A170, F247, S276, R277, Q278, L279, A280, R281, V282, A302, W303, S306, R307, H308, I309, G311, R317, and W357 of a human T1R1, 3) L46, H47, S48, G49, C50, L51, S67, F68, N69, E70, H71, C106, S107, D108, D147, S148, R151, Y169, A170, Y220, F247, S248, S275, S276, R277, Q278, L279, A280, R281, V282, F283, F284, E285, E301, A302, W303, S306, R307, H308, I309, T310, G311, V312, P313, R317, K354, W357, K377, K379, M383, and S385 of a human T1R1, 4) L46, H47, S48, G49, C50, L51, S67, F68, N69, E70, H71, C106, S107, D108, D147, S148, T149, N150, R151, Y169, A170, A171, S172, S173, D192, N195, D218, Y220, S276, R277, E301, A302, F247, S248, S275, S276, R277, Q278, L279, A280, R281, V282, F283, F284, E285, E301, A302, W303, S306, R307, H308, I309, T310, G311, V312, P313, R317, K354, W357, K377, K379, M383, and S385 of a human T1R1, 5) S172, Y220, D192, E301, and T149 of a human T1R1, and 6) a combination thereof.

49. The chemosensory receptor ligand modifier of claim 47, wherein it interacts with T1R1 Venus flytrap domain via a group of amino acid L46, H47, S48, G49, C50, L51, S67, F68, N69, E70, H71, C106, S107, D108, D147, S148, R151, Y169, A170, Y220, F247, S248, S275, S276, R277, Q278, L279, A280, R281, V282, F283, F284, E285, E301, A302, W303, S306, R307, H308, I309, T310, G311, V312, P313, R317, K354, W357, K377, K379, M383, and S385 of a human T1R1 and wherein the chemosensory receptor ligand modifier enhances the activity of a chemosensory receptor ligand.

50. The chemosensory receptor ligand modifier of claim 47, wherein it interacts with T1R1 Venus flytrap domain via a group of amino acid H47, S48, G49, C50, S67, F68, N69, E70, H71, S107, D147, S148, A170, F247, S276, R277, Q278, L279, A280, R281, V282, A302, W303, S306, R307, H308, I309, G311, R317, and W357 of a human T1R1 and wherein the chemosensory receptor ligand modifier enhances the activity of a chemosensory receptor ligand.

51. The chemosensory receptor ligand modifier of claim 47, wherein it is a chemosensory receptor ligand enhancer and has structural Formula (I): 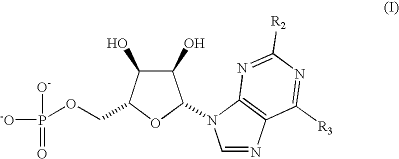
or a salt, hydrate or solvate thereof, wherein:
R₂ is hydrogen, —NR₄R₅ or —NR₄C(O)R₅;
R₄ and R₅ are independently hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl or substituted heteroarylalkyl;
R₃ is hydroxyl, —NR₆R₇, —NR₆C(O)R₇ or —S(O)ₐR₆;
R₆ and R₇ are independently hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl or substituted heteroarylalkyl; and
a is 0, 1 or 2;
provided that when R₂ is hydrogen then R₃ is not hydroxyl; and
when R₂ is —NH₂ then R₃ is not hydroxyl.

52. The ligand modifier of claim 51, wherein when R₂ is —NH₂ then R₃ is not —SH; when R₃ is hydrogen, R₂ is —NR₄R₅ and R₄ is hydrogen then R₅ is not hydrogen, alkanyl, (C₂-C₅) alkenyl, substituted alkyl, heteroalkanyl, phenyl, para-aminophenyl, benzyl, homobenzyl, para-azidohomobenzyl, 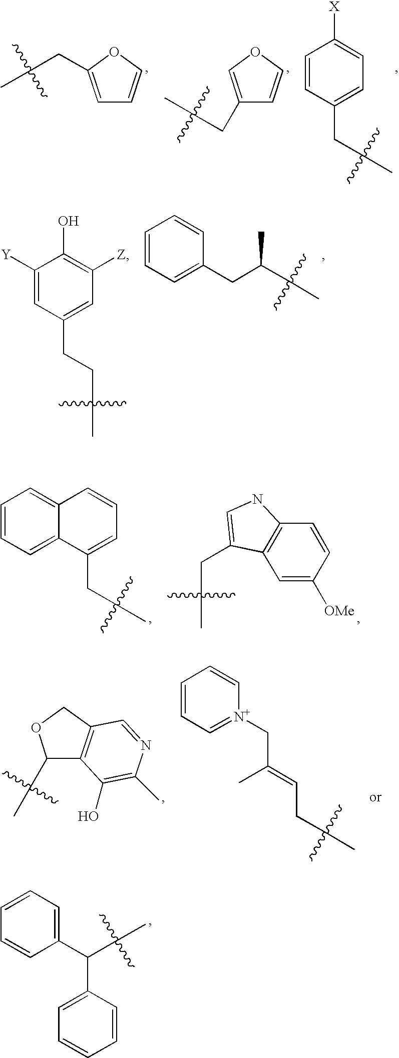
where X is —NH₂, —NO₂, —NHC(O)CH₃ or —NHC(O)CH₂Br and Y and Z are independently hydrogen or iodine;
when R₃ is hydrogen, R₂ is —NR₄R₅ and R₄ is methyl, n-butyl, 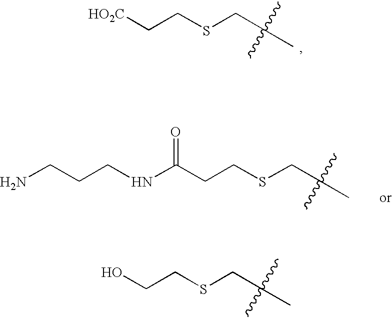
then R₅ is not methyl, n-butyl, α-napthyl, substituted alkyl, 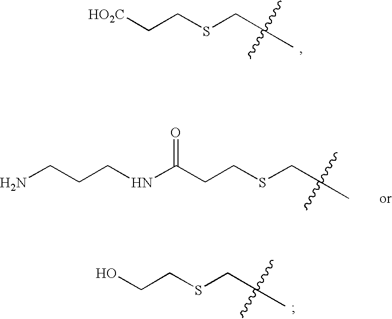
when R₃ is hydrogen and R₂ is —SR₆, then R₆ is not methyl, butyl, para-nitrobenzyl, para-aminobenzyl, 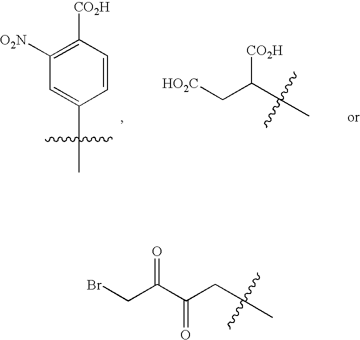
when R₂ is hydroxyl then R₃ is not 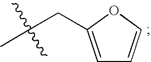
when R₃ is hydroxyl, R₂ is —NR₄R₅ and R₄ is hydrogen then R₅ is not hydrogen, methyl, butyl, C₁-C₃ substituted alkyl, —(CH₂)₄Ph, —(CH₂)₃SMe, 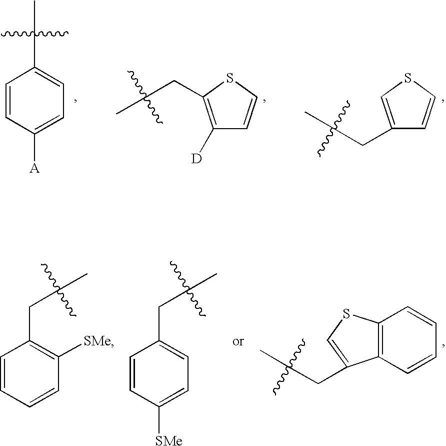
A is methyl, n-butyl, fluorine or bromine and D is hydrogen, methyl, ethyl or nitro; when R₃ is hydroxyl, R₂ is —NR₄R₅ and R₄ is methyl then R₅ is not methyl;
when R₃ is hydroxyl, R₂ is —NR₄C(O)R₅ and R₄ is hydrogen then R₅ is not phenyl, 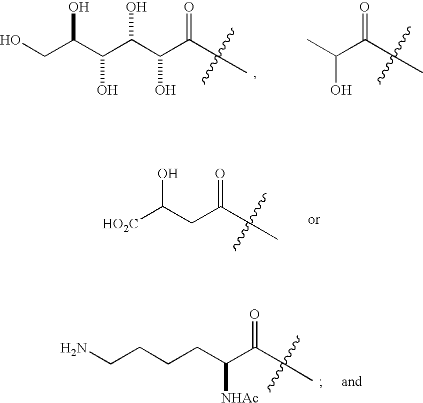
when R₃ is —NH₂ then R₂ is not dimethylamino, methylamino, ethylamino, butylamino, acetamido or para-n-butylaniline.

53. The ligand modifier of claim 51, wherein when R₂ is hydrogen then R₃ is not —NH₂.

54. The ligand modifier of claim 51, wherein when R₂ is —NH₂ then R₃ is not —NH₂.

55. The ligand modifier of claim 51, wherein R₄ and R₅ are independently hydrogen, alkyl, substituted alkyl, aryl, substituted aryl, arylalkyl, substituted arylalkyl, cycloalkyl, heteroalkyl, heteroarylalkyl or substituted heteroarylalkyl.

56. The ligand modifier of claim 51, wherein R₂ is hydrogen, —NH₂, or —NHC(O)R₅ and R₅ is alkyl, substituted alkyl, aryl, substituted aryl, heteroaryl or substituted heteroaryl.

57. The ligand modifier of claim 51, wherein R₃ is hydroxyl, —NR₆R₇, —NHC(O)R₇ or —SR₆, R₆ is heteroarylalkyl, aryl, substituted aryl, arylalkyl, substituted arylalkyl, alkyl, cycloalkyl, heteroalkyl or substituted cycloheteroalkyl and R₇ is alkyl, alkyl, aryl or substituted aryl.

58. The ligand modifier of claim 51 wherein R₂ is hydrogen, —NH₂, or —NHC(O)R₅, R₅ is alkyl, substituted alkyl, aryl, substituted aryl, heteroaryl, substituted heteroaryl and R₃ is hydroxyl, —NR₆R₇, —NHC(O)R₇ or —SR₆, R₆ is heteroarylalkyl, aryl, substituted aryl, arylalkyl, substituted arylalkyl, alkyl, cycloalkyl, heteroalkyl or substituted cycloheteroalkyl and R₇ is alkyl, alkyl, aryl or substituted aryl.

59. The ligand modifier of claim 51, wherein R₂ is —NH₂ and R₃ is —NH₂.

60. The ligand modifier of claim 51, wherein R₂ is —NH₂ and R₃ is —NHR₇ and R₇ is heteroarylalkyl,

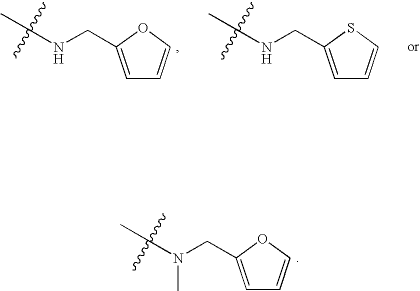

61. The ligand modifier of claim 51, wherein R₃ is hydroxyl and R₂ is —NHC(O)R₅ and R₅ is heteroaryl or substituted heteroaryl.

62. The ligand modifier of claim 61, wherein R₅ is 2-furanyl or 2-thienyl.

63. The ligand modifier of claim 51, wherein R₂ is —NH₂, R₃ is —NR₆R₇, R₆ is hydrogen, R₇ is 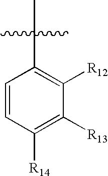
and R₁₂, R₁₃ and R₁₄ are independently hydrogen, alkoxy, alkyl or halo.

64. The ligand modifier of claim 63, wherein R₁₂, R₁₃ and R₁₄ are independently hydrogen, methoxy, methyl or fluorine.

65. The ligand modifier of claim 63, wherein R₁₂ is hydrogen, methoxy, methyl or fluoro and R₁₃ and R₁₄ are hydrogen.

66. The ligand modifier of claim 63, wherein R₉ is methoxy, methyl or fluoro and R₈ and R₁₀ are hydrogen.

67. The ligand modifier of claim 63, wherein R₁₀ is methoxy, methyl or fluoro and R₈ and R₉ are hydrogen.

68. The ligand modifier of claim 51, wherein R₂ is —NH₂, R₃ is —NR₆R₇, R₆ is hydrogen or methyl, R₇ is 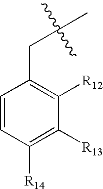
and R₁₂, R₁₃ and R₁₄ are independently hydrogen, alkoxy, alkyl or halo.

69. The ligand modifier of claim 68, wherein R₁₂, R₁₃ and R₁₄ are independently hydrogen, methoxy, methyl or fluorine.

70. The ligand modifier of claim 68, wherein R₆ is hydrogen or methyl and R₁₂, R₁₃ and R₁₄ are hydrogen.

71. The ligand modifier of claim 68, wherein R₆ is hydrogen, R₁₂ is methoxy, methyl or fluoro and R₁₃ and R₁₄ are hydrogen.

72. The ligand modifier of claim 68, wherein R₆ is hydrogen, R₁₃ is methoxy, methyl or fluoro and R₁₂ and R₁₄ are hydrogen.

73. The ligand modifier of claim 68, wherein R₆ is hydrogen, R₁₄ is methoxy, methyl or fluoro and R₁₂ and R₁₃ are hydrogen.

74. The ligand modifier of claim 51, wherein R₂ is —NH₂ and R₃ is —NR₆R₇, R₆ and R₇ are independently hydrogen, alkyl or cycloalkyl or alternatively, R₆ and R₇ together with the atoms to which they are attached form a cycloheteroalkyl ring.

75. The ligand modifier of claim 74, wherein R₆ is hydrogen and R₇ is alkyl or cycloalkyl.

76. The ligand modifier of claim 74, wherein R₆ is hydrogen and R₇ is isopropyl, n-butyl, n-pentyl, cyclopropyl or cyclopentyl.

77. The ligand modifier of claim 74, wherein R₆ and R₇ together with the atoms to which they are attached form a piperidinyl or pyrrolidinyl ring.

78. The ligand modifier of claim 51, wherein R₃ is —OH, R₂ is —NHC(O)R₅ and R₅ is alkyl, substituted alkyl, aryl, substituted aryl or cycloalkyl.

79. The ligand modifier of claim 78, wherein R₅ is 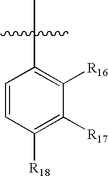
and R₁₆, R₁₇ and R₁₈ are independently hydrogen, alkoxy, alkyl, substituted alkyl or halo.

80. The ligand modifier of claim 79, wherein R₁₆, R₁₇ and R₁₈ are independently hydrogen, fluoro, methoxy, methyl or trifluoromethyl.

81. The ligand modifier of claim 79, wherein R₁₆ is methoxy or fluoro and R₁₇ and R₁₈ are hydrogen.

82. The ligand modifier of claim 79, wherein R₁₇ is methoxy or methyl and R₁₆ and R₁₈ are hydrogen.

83. The ligand modifier of claim 79, wherein R₁₈ is methoxy or trifluoromethyl and R₁₆ and R₁₇ are hydrogen.

84. The ligand modifier of claim 79, wherein R₅ is isopropyl, n-butyl, cyclohexyl or —CH₂OPh.

85. The ligand modifier of claim 51, wherein R₂ is hydrogen and R₃ is —NR₆R₇.

86. The ligand modifier of claim 85, wherein R₆ is hydrogen, alkyl or arylalkyl and R₇ is aryl, substituted aryl, arylalkyl, substituted arylalkyl, heteroarylalkyl, heteroalkyl, cycloalkyl or substituted cycloheteroalkyl.

87. The ligand modifier of claim 85, wherein —NR₆R₇ is 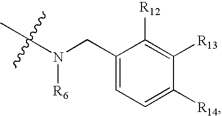
R₆ is hydrogen, alkyl or arylalkyl and R₁₂, R₁₃ and R₁₄ are independently hydrogen, alkyl, alkoxy or halo.

88. The ligand modifier of claim 87, wherein R₆ is hydrogen, methyl or benzyl and R₁₂, R₁₃ and R₁₄ are hydrogen.

89. The ligand modifier of claim 87, wherein R₆ is hydrogen, R₁₂ is methyl and R₁₃ and R₁₄ are hydrogen.

90. The ligand modifier of claim 87, wherein R₆ is hydrogen, R₁₃ is methyl, methoxy or fluorine and R₁₂ and R₁₄ are hydrogen.

91. The ligand modifier of claim 87, wherein R₆ is hydrogen, R₁₄ is methoxy or fluorine and R₁₂ and R₁₃ are hydrogen.

92. The ligand modifier of claim 85, wherein R₆ is hydrogen or alkyl and R₇ is alkyl, heteroalkyl, cycloalkyl, substituted cycloheteroalkyl, arylalkyl or heteroarylalkyl or alternatively, R₆ and R₇ together with the atoms to which they are attached form a cycloheteroalkyl ring.

93. The ligand modifier of claim 92, wherein R₆ and R₇ are n-propyl.

94. The ligand modifier of claim 92, wherein R₆ is methyl and R₇ is

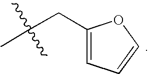

95. The ligand modifier of claim 92, wherein R₆ is hydrogen and R₇ is methyl, ethyl, n-butyl or n-octyl.

96. The ligand modifier of claim 92, wherein R₆ is hydrogen and R₇ is

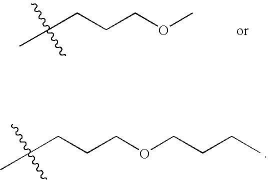

97. The ligand modifier of claim 92, wherein R₆ is hydrogen and R₇is

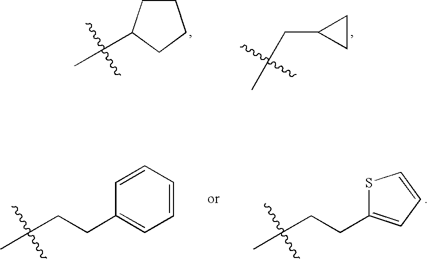

98. The ligand modifier of claim 92, wherein R₆ and R₇ together with the atoms to which they are attached form a cycloheteroalkyl ring.

99. The ligand modifier of claim 98, wherein R₆ and R₇ together with the atoms to which they are attached form:

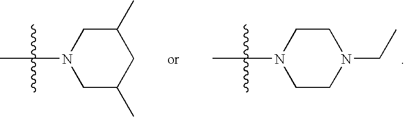

100. The ligand modifier of claim 85, wherein R₆ is hydrogen, R₇ is 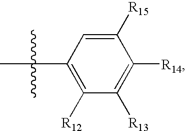
R₁₂, R₁₃, R₁₄ and R₁₅ are independently alkyl, —CH₃, alkoxy, —OCH₃, —OC₂H₅, halo, —F, —Cl, or —Br, —NHCOR₁₂.

101. The ligand modifier of claim 100, wherein R₁₂ is methyl or fluoro and R₁₃, R₁₄ and R₁₅ are hydrogen.

102. The ligand modifier of claim 100, wherein R₁₃ is methyl, methoxy, fluoro, bromo or —NHCOCH₃ and R₁₂, R₁₄ and R₁₅ are hydrogen.

103. The ligand modifier of claim 100, wherein R₁₄ is methyl, n-butyl, methoxy, ethoxy, fluoro or chloro and R₁₂, R₁₃ and R₁₅ are hydrogen.

104. The ligand modifier of claim 100, wherein R₁₃ and R₁₄ are methoxy, fluoro or chloro and R₁₂ and R₁₅ are hydrogen.

105. The ligand modifier of claim 100, wherein R₁₃ is chloro, R₁₄ is methyl and R₁₂ and R₁₅ are hydrogen.

106. The ligand modifier of claim 100, wherein R₁₃ and R₁₅ are chloro and R₁₂ and R₁₄ are hydrogen.

107. The ligand modifier of claim 51, wherein R₂ is hydrogen and R₃ is —NHCOR₇.

108. The ligand modifier of claim 107, wherein R₇ is alkyl, aryl, substituted aryl or heteroaryl.

109. The ligand modifier of claim 107, wherein R₇ is methyl, n-propyl or isopropyl.

110. The ligand modifier of claim 107, wherein R₇ is 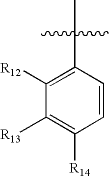
and R₁₂, R₁₃ and R₁₄ are independently hydrogen, alkoxy, —OCH₃, alkyl, —CH₃, halo or —F.

111. The ligand modifier of claim 107, wherein R₁₂ is methyl, methoxy or flourine and R₁₃ and R₁₄ are hydrogen.

112. The ligand modifier of claim 107, wherein R₁₃ is methyl, methoxy or flourine and R₁₂ and R₁₄ are hydrogen.

113. The ligand modifier of claim 107, wherein R₁₂ is methoxy and R₁₃ and R₁₄ are hydrogen.

114. The ligand modifier of claim 107, wherein R₇ is 2-furanyl.

115. The ligand modifier of claim 107, wherein R₂ is hydrogen and R₃ is —SR₆.

116. The ligand modifier of claim 115, wherein R₆ is alkyl, heteroalkyl, arylalkyl or substituted arylalkyl.

117. The ligand modifier of claim 115, wherein R₆ is 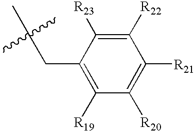
and R₁₉, R₂₀, R₂₁, R₂₂ and R₂₃ are independently alkyl, alkoxy, halo or cyano.

118. The ligand modifier of claim 117, wherein R₁₉, R₂₀, R₂₁, R₂₂ and R₂₃ are independently methyl, methoxy, fluoro, chloro, bromo or cyano.

119. The ligand modifier of claim 117, wherein R₁₉ is hydrogen, methyl, methoxy, fluoro, chloro, bromo or cyano and R₂₀, R₂₁, R₂₂ and R₂₃ are hydrogen.

120. The ligand modifier of claim 117, wherein R₂₀ is methyl, methoxy, fluoro or cyano and R₁₉, R₂₁, R₂₂ and R₂₃ are hydrogen.

121. The ligand modifier of claim 117, wherein R₂₁ is methoxy, fluoro or chloro and R₁₉, R₂₀, R₂₂ and R₂₃ are hydrogen.

122. The ligand modifier of claim 117, wherein R₂₀ and R₂₁ are methyl and R₁₉, R₂₂ and R₂₃ are hydrogen.

123. The ligand modifier of claim 117, wherein R₁₉ and R₂₂ are methyl and R₂₀, R₂₁ and R₂₃ are hydrogen.

124. The ligand modifier of claim 117, wherein R₁₉ and R₂₁ are chloro and R₂₀, R₂₂ and R₂₃ are hydrogen.

125. The ligand modifier of claim 117, wherein R₁₉ is chloro, R₂₃ is fluoro and R₂₀, R₂₁ and R₂₂ are hydrogen.

126. The ligand modifier of claim 117, wherein R₆ is hydrogen, methyl, isopropyl, isobutyl, or

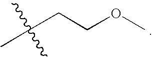

127. The ligand modifier of claim 117, wherein R₆ is

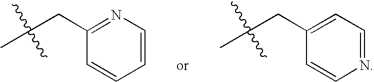

128. The ligand modifier of claim 51, wherein R₂ is —NHCOR₅, R₃ is —OH and R₅ is aryl, substituted aryl, heteroaryl or substituted heteroaryl.

129. The ligand modifier of claim 128, wherein R₅ is

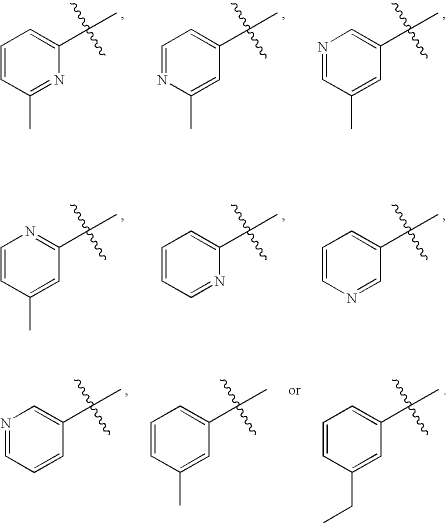

130. The chemosensory receptor ligand modifier of claim 47, wherein it is a chemosensory receptor ligand enhancer and has structural Formula (II): 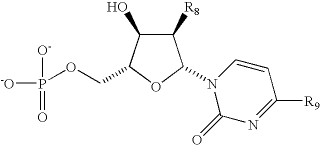
or a salt, hydrate or solvate thereof, wherein:
R₈ is hydrogen or hydroxyl;
R₉ is —NR₁₀C(O)R₁₁;
R₁₀ is hydrogen, substituted alkyl, aryl, substituted aryl, arylalkyl, substituted arylalkyl, heteroalkyl, substituted heteroalkyl, heteroaryl, substituted heteroaryl, heteroarylalkyl or substituted heteroarylalkyl; and
R₁₁ is hydrogen, (C₁-C₁₀)alkyl, substituted alkyl, aryl, substituted aryl, arylalkyl, substituted arylalkyl, heteroalkyl, substituted heteroalkyl, heteroaryl, substituted heteroaryl, heteroarylalkyl or substituted heteroarylalkyl.

131. The ligand modifier of claim 130, wherein R₉ is not 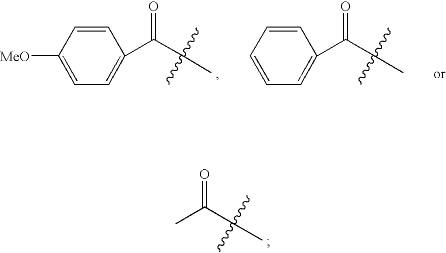
when R₈ is hydrogen then R₉ is not 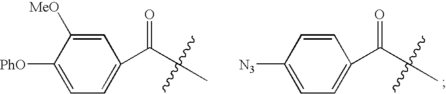
and when R₈ is hydroxyl then R⁹ is not

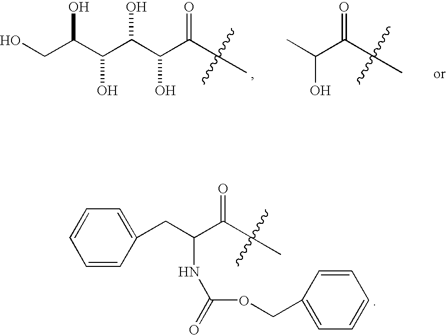

132. The ligand modifier of claim 130, wherein R₁₀ is hydrogen and R₁₁ is heteroaryl, alkyl, substituted alkyl, aryl or substituted aryl.

133. The ligand modifier of claim 130, wherein R₈ is hydrogen, R₁₀ is hydrogen and R₁₁ is alkyl, substituted alkyl, aryl or substituted aryl.

134. The ligand modifier of claim 130, wherein R₁₁ is isopropyl, t-butyl, —CH₂OPh or 3-methylphenyl.

135. The ligand modifier of claim 130, wherein R₈ is hydrogen, R₁₀ is hydrogen and R₁₁ is 2-thienyl.

136. The ligand modifier of claim 130, wherein R₈ is hydroxyl, R₁₀ is hydrogen and R₁₁ is aryl or substituted aryl.

137. The ligand modifier of claim 136, wherein R₁₁ is phenyl, 3-methylphenyl or 4-methoxyphenyl.

138. The chemosensory receptor ligand modifier of claim 47, wherein it is a chemosensory receptor ligand enhancer and has structural Formula (III): 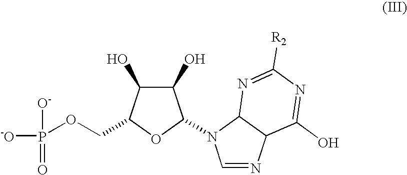
or a salt, hydrate or solvate thereof, wherein:
R₂ is hydrogen, —NR₄R₅ or —NR₄C(O)R₅; and
R₄ and R₅ are independently hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl or substituted heteroarylalkyl.

139. The ligand modifier of claim 138, wherein R₂ is hydrogen or —NH₂.

140. The chemosensory receptor ligand modifier of claim 47, wherein it is a chemosensory receptor ligand enhancer and has structural Formula (IV): 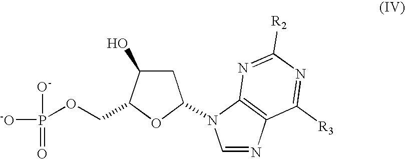
or a salt, hydrate or solvate thereof, wherein:
R₂ is hydrogen, —NR₄R₅ or —NR₄C(O)R₅;
R₄ and R₅ are independently hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl or substituted heteroarylalkyl;
R₃ is hydroxyl, —NR₆R₇ or —NR₆C(O)R₇;
R₆ and R₇ are independently hydrogen, alkyl, substituted alkyl, aryl, substituted aryl, arylalkyl, substituted arylalkyl, heteroalkyl, substituted heteroalkyl, heteroaryl, substituted heteroaryl, heteroarylalkyl or substituted heteroarylalkyl; and
a is 0, 1 or 2;
provided that when R₂ is hydrogen then R₃ is not hydroxyl; and
when R₂ is —NH₂ then R₃ is not hydroxyl.

141. The ligand modifier of claim 140, wherein R₂ is hydrogen or —NR₄R₅ and R₃ is hydroxyl or —NR₆R₇.

142. The ligand modifier of claim 140, wherein R₂ is hydrogen or —NH₂ and R₃ is hydroxyl or —NH₂.

143. The chemosensory receptor ligand modifier of claim 47 in a comestible composition.

144. The chemosensory receptor ligand modifier of claim 47 in a food or beverage product.

145. The chemosensory receptor ligand modifier of claim 47 in a medicinal composition as a non-active ingredient.

146. The chemosensory receptor ligand modifier of any one of claims 51, 130, 138 or 140 in a medicinal composition as an active ingredient.

147. A comestible composition comprising between about 0.0001 ppm to about 10 ppm of a ligand modifier of any one of claims 51, 130, 138 or 140.

148. A comestible or medicinal composition comprising between about 0.01 ppm to about 100 ppm of a ligand modifier of any one of claims 51, 130, 138 or 140 and at least a umami flavor entity.

149. A composition comprising between about 10 ppm to about 100,000 ppm of a ligand modifier of any one of claims 51, 130, 138 or 140.

150. A method of enhancing the umami taste of a comestible or medicinal product comprising contacting a comestible or medicinal product or precursors thereof with a ligand modifier of any one of claims 51, 130, 138 or 140 to form a modified comestible or medicinal product, wherein the modified comestible or medicinal product comprises at least about 0.001 ppm of the ligand modifier.

151. A method of treating a condition associated with a chemosensory receptor comprising administering to a subject in need of such treatment an effective amount of an entity selected from the group consisting of a chemosensory receptor modifier, chemosensory receptor ligand modifier, and a combination thereof, wherein the entity interacts with an interacting site of the chemosensory receptor.

152. The method of claim 151, wherein the condition associated with a chemosensory receptor is taste.

153. The method of claim 151, wherein the condition associated with a chemosensory receptor is a condition associated with gastrointestinal system or metabolic disorders.

154. The method of claim 151, wherein the condition associated with a chemosensory receptor is a condition associated with a functional gastrointestinal disorder.

155. The method of claim 151, wherein the condition associated with a chemosensory receptor is a condition associated with cells expressing a T1R.

156. The method of claim 151, wherein the condition associated with a chemosensory receptor is a condition associated with hormone-producing cells that express a T1R.
